(19) United States
(12) Patent Application Publication
(10) Pub. No.: US 2026/0083778 A1
(43) Pub. Date: Mar. 26, 2026

(54) GENERATION OF CD4 T CELLS

(71) Applicant: BOSTON MEDICAL CENTER CORPORATION, Boston, MA (US)

(72) Inventors: Gustavo MOSTOSLAVSKY, West Roxbury, MA (US); Julian AMIRAULT, Jamaica Plain, MA (US)

(73) Assignee: BOSTON MEDICAL CENTER CORPORATION, Boston, MA (US)

(21) Appl. No.: 19/334,256

(22) Filed: Sep. 19, 2025

Related U.S. Application Data
(60) Provisional application No. 63/696,952, filed on Sep. 20, 2024.

(51) Int. Cl. A61K 35/17; A61K 40/11; A61K 40/31; A61K 40/42; A61P 37/04; C07K 14/73; C12N 5/00; C12N 5/0783

(52) U.S. Cl. CPC A61K 35/17; A61K 40/11; A61K 40/31; A61K 40/421; A61P 37/04; C07K 14/70514; C12N 5/0018; C12N 5/0636; C12N 2501/155; C12N 2501/165; C12N 2501/2302; C12N 2501/2307; C12N 2501/51; C12N 2501/515; C12N 2501/727; C12N 2506/1369; C12N 2510/00; C12N 2533/52

(57) ABSTRACT

The technology described herein is directed to methods of generating CD4+CD8− single-positive T cells from CD4+CD8+ double positive T progenitor cells using differentiation in the presence of interleukin-7 (IL-7) and an anti-CD3 agent and in absence of Notch ligand and an anti-CD28 agent. Also described herein are CD4+CD8− single-positive T cells made by the methods described herein, which can be used for therapeutic applications.

### CROSS-REFERENCE TO RELATED APPLICATIONS

This application claims benefit under 35 U.S.C. § 119 of U.S. Provisional Application No. 63/696,952 filed Sep. 20, 2024, the contents of which are incorporated herein by reference in their entirety.

### SEQUENCE LISTING

The instant application contains a Sequence Listing which has been submitted in XML format via Patent Center and is hereby incorporated by reference in its entirety. Said XML copy; created on Sep. 18, 2025, is named 701586-000147WOPT_USPT_SL.xml and is 14,544 bytes in size.

### TECHNICAL FIELD

The technology described herein relates to methods for generating CD4+ T cells from pluripotent stem cells, such as induced pluripotent stem cells (iPSCs), including for use in therapeutic applications.

### BACKGROUND

T cells originate as hematopoietic progenitor cells which migrate from the bone marrow to the thymus. Transition from hematopoietic progenitors to a CD4/CD8 double negative followed by a CD4/CD8 double positive lymphoid population takes place in the Notch ligand-rich environment of the thymic cortex, where the cells are maintained by survival cytokine IL-7. Double positive progenitor cells are then signaled to mature to single positive thymocytes by T cell receptor (TCR) binding to major histocompatibility complexes (MHCs) presented by thymic epithelial cells in a process termed “positive selection”. The initiation of positive selection occurs in the thymic cortex, and positively selected DP thymocytes migrate through the cortico-medullary junction into the medulla. After migration to the medulla, the single positive (SP) semi-mature T cells undergo negative selection in which highly self-reactive clones undergo apoptosis, with the exception of some strongly signaled CD4+ clones, which survive and are diverted to an anti-inflammatory regulatory T cell fate.

Despite work implicating strength and duration of TCR signaling alongside co-receptor or cytokine signals in functional lineage differentiation and commitment, the mechanisms resulting in the divergence of these two fundamental T cell lineages, CD8+ vs CD4+, remain in question (see e.g., Park et al., Nature Immunology 11, 257-264, 2010: Xing et al., Nature Immunology 17, 565-373, 2016; Karimi et al., Nature Communications 12, 99, 2021: Irla, Annual Review of Immunology 40, 95-119, 2022; Martinez and Hogquist, Current Opinion in Immunology 84, 102389-102389, 2023). IL-7 is an established requirement for CD8 SP T cell survival, and both CD4 and CD8 SP cells require the binding of the TCR to MHC (MHC II and MHC I respectively) loaded with a compatible antigen for lineage commitment.

T cells are the basis of multiple promising cell therapies, from Chimeric Antigen Receptor (CAR) T cell approaches which target cancers to T regulatory cell and CAR T regulatory anti-inflammatory treatments for inflammatory conditions. Conventional CAR T immunotherapy approaches use either autologous T cells derived from the patient being treated or allogeneic T cells taken from healthy donors. A key drawback of autologous CAR T therapy is time and cost to treatment, while donor-to-donor variation in starting T cell material can result in differences in outcome for patients. Not unexpectedly, for CAR T therapies, the ratio of CD4+ to CD8+ T cells alongside the presence or absence of specific T cell subpopulations have been connected to the effectiveness of treatment response. Allogeneic CAR T can be easily prepared and banked prior, but the risk of immune rejection is notable; groups are currently working on universal donor edits to cell preparations reducing immunogenicity. An alternative cell source with lower cost and rapid production when compared to autologous or allogeneic sources could rely on the generation of T cells from induced Pluripotent Stem Cells (iPSC). The same edits, including MHC knockdown and overexpression of anti-inflammatory proteins to prevent allogeneic rejection are easily applicable to iPSC derived T cells. Currently. CD8+ iPSC T cells can be generated at scale needed for its use as a therapeutic agent: however, an effective and scalable iCD4+ T helper cell protocol so far has been out of reach. CD4+ T cell populations have been shown to be highly relevant in current CAR T immunotherapies, providing important regulatory support to CD8+ T cells.

While robust generation of the CD4+/CD8+ double positive (DP) progenitor cells from iPSCs have been demonstrated, most protocols yield only CD8+ cytotoxic lymphocytes (Maeda et al., Cancer research 76, 6839-6850, 2016; Wang et al., Nature Biomedical Engineering 5, 429-440, 2021; Heinze et al., Stem Cell Reports 17, 2610-2628, 2022). CD4+ T cell cytokines are important for many effective T cell therapies. There is thus great need for protocols to specifically differentiate CD4+ T cells.

### SUMMARY

The present disclosure provides a highly scalable, feeder-free platform for the generation of functional iPSC-derived CD4+ T cells, and details their stimulation-specific combinational expression of cytokines and cytotoxic effector molecules at single cell resolution using CyTOF alongside transcriptional signatures using single cell RNA sequencing. Specifically, the present disclosure relates to methods and compositions for producing a population of CD4⁺CD8⁻ single-positive T cells from precursor progenitor cells, without the requirement for Notch ligand-mediated signaling or an anti-CD28 agent. In some embodiments, the technology described herein provides a protocol in which CD4 CD8; double-positive T progenitor cells are cultured in the absence of exogenous Notch ligand and an anti-CD28 agent, and in a medium comprising interleukin-7 (approximately 10 ng/mL) and an anti-CD3 agent, to direct differentiation into CD4⁺CD8⁻ single-positive T cells.

In particular, the anti-CD3 agent specifically binds and activates the CD3 complex and may comprise an anti-CD3 antibody (for example, clone OKT3 at about 5 μg/mL). Progenitor cells can be maintained for at least 14 days on a substrate coated with fibronectin or a recombinant embodiment thereof at a coating density of at least 10 μg/mL. Following this culture period, the resulting CD4 CD8 single-positive T cells can be harvested and further expanded in a medium containing the same or different anti-CD3 agent, an anti-CD28 agent that binds and activates CD28 (which may be present, e.g., together with the anti-CD3 agent, at approximately 25 μg/mL or delivered as a bispecific agent), and interleukin-2 at about 200 U/mL.

Accordingly: expansion over a period of 14 to 140 days yields at least two to six population doublings and a cell population comprising at least 40%-60% CD4⁺CD8⁻ single-positive T cells. The expanded cells express surface markers and transcription factors consistent with mature T cells, including CD3, TCRαβ, CD62L, CD5, CCR7, MHC I, CD69, CD25, ThPOK, and TRAC. Upon in vitro stimulation, at least 90% of the CD4⁺CD8⁻ single-positive T cells can secrete multiple cytokines and effector molecules, such as IFNγ, TNFα, IL-2, IL-4, IL-5, IL-6, IL-8, IL-9, IL-10, IL-13, IL-17A, IL-17F, IL-22, amphiregulin, granzyme B, MIP1α, and MIP1β, demonstrating robust functional competency. Moreover, these cells may be isolated and cryopreserved with post-thaw viabilities suitable for downstream applications.

Another aspect relates to a method of producing CD4⁺CD8⁻ double-positive T progenitors prior to the single-positive differentiation. In some embodiments, a population of CD34⁺ hematopoietic stem and progenitor cells (HSPCs) is first differentiated on a Notch ligand-coated substrate (for example, Delta-like ligand 1 and/or Delta-like ligand 4) in a medium containing interleukin-7 for at least 28 days to generate CD4⁺CD8⁻; double-positive T progenitor cells. Optionally: the process can begin with induced pluripotent stem cells (iPSCs) that have been cultured for at least 12 days in a medium comprising BMP-4. VEGF, and a glycogen synthase kinase-3β inhibitor, and may include genetic modification to express an exogenous polypeptide such as a chimeric antigen receptor (CAR) with a CD19-specific single-chain variable fragment, a CD35 signaling domain, and a CD28 and/or 4-1BB costimulatory domain, integrated at the TRAC or AAVS1 locus.

Unlike protocols that rely on sustained Notch activation or CD28 co-stimulation using an anti-CD28 agent, the present methods allow differentiation of CD4⁺CD8⁻ single-positive T cells under minimal exogenous signaling conditions. As a result, the disclosed methods can streamline cell manufacturing by reducing dependency on feeder cells. Notch ligands, and complex selection procedures.

Another embodiment provides regulatory T cell (Treg) populations obtained by further culturing the CD4⁺CD8⁻ single-positive T cells in a medium containing TGF-β, interleukin-2, retinoic acid, and rapamycin. Compositions comprising the resulting T cell populations, either alone or formulated with a pharmaceutically acceptable carrier, are also provided. In some aspects, a method of adoptive cell transfer comprises administering an effective amount of the T cell population or composition to a subject, where the cells may be autologous or allogeneic to the subject. Additionally, the CD4⁺CD8⁻ single-positive T cells described herein can be employed in in vitro drug-screening assays and immunological assays, thereby broadening their applicability in research and therapeutic contexts.

In one aspect, described herein is a method of producing a population of CD4+CD8− single-positive (SP) T cells, the method comprising: culturing a population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells.

In some embodiments of any of the aspects, the anti-CD3 agent specifically binds and activates CD3 intracellular signalling.

In some embodiments of any of the aspects, the anti-CD3 agent is not bound to a substrate.

In some embodiments of any of the aspects, the anti-CD3 agent comprises an anti-CD3 antibody or an antigen binding domain thereof.

In some embodiments of any of the aspects, the anti-CD3 antibody is selected from Table 4 (e.g., OKT3, Otelixizumab, Teplizumab, HIT3a, UCHT1, 17A2, 145-2C11, or Foralumab).

In some embodiments of any of the aspects, the anti-CD3 agent comprises an anti-CD3 aptamer.

In some embodiments of any of the aspects, the anti-CD3 aptamer is selected from Table 5.

In some embodiments of any of the aspects, the anti-CD3 agent is present at a concentration of about 5 μg/mL.

In some embodiments of any of the aspects, the IL-7 is present at a concentration of about 10 ng/mL.

In some embodiments of any of the aspects, the population of DP T progenitor cells are cultured for at least 14 days.

In some embodiments of any of the aspects, the population of DP T progenitor cells are cultured with a substrate coated with fibronectin or a recombinant variant thereof.

In some embodiments of any of the aspects, the fibronectin or recombinant variant thereof is coated on the substrate at a concentration of at least 10 μg/mL.

In some embodiments of any of the aspects, the method further comprises culturing the population of CD4+CD8− SP T cells in a medium comprising: the anti-CD3 agent, the anti-CD28 agent, and interleukin-2 (IL-2).

In some embodiments of any of the aspects, the anti-CD28 agent specifically binds and activates CD28 intracellular signalling.

In some embodiments of any of the aspects, the anti-CD28 agent comprises an anti-CD28 antibody or an antigen binding domain thereof.

In some embodiments of any of the aspects, the anti-CD28 antibody is selected from Table 6.

In some embodiments of any of the aspects, the anti-CD28 agent comprises an anti-CD28 aptamer.

In some embodiments of any of the aspects, the anti-CD28 aptamer is selected from Table 7.

In some embodiments of any of the aspects, the anti-CD3 agent and the anti-CD28 agent are present at a concentration of about 25 μg/mL.

In some embodiments of any of the aspects, the anti-CD3 agent and the anti-CD28 agent are comprised by a single bispecific agent.

In some embodiments of any of the aspects, the anti-CD3 agent and the anti-CD28 agent are linked to a substrate.

In some embodiments of any of the aspects, the substrate is a magnetic bead or a cell culture vessel.

In some embodiments of any of the aspects, the anti-CD3 agent and the anti-CD28 agent are in a tetrameric complex, optionally wherein the anti-CD3 agent and the anti-CD28 agent comprise anti-CD3 antibodies and anti-CD28 antibodies in tetrameric complexes.

In some embodiments of any of the aspects, the anti-CD3 agent and the anti-CD28 agent are selected from Table 8.

In some embodiments of any of the aspects, the IL-2 is present at a concentration of about 200 U/mL.

In some embodiments of any of the aspects, the population of CD4+CD8− SP T cells is cultured for at least 14 days to at most 140 days

In some embodiments of any of the aspects, the culturing increases proliferation of the population of CD4+CD8− SP T cells.

In some embodiments of any of the aspects, the population of CD4+CD8− SP T cells undergo at least 2, at least 3, at least 4, at least 5, or at least 6 population doublings.

In some embodiments of any of the aspects, the population of CD4+CD8− SP T cells comprises at least 40%, at least 50%, at least 60% or more CD4+CD8− SP T cells; and/or wherein the population of CD4+CD8− SP T cells comprises at least 1×10⁷, at least 1×10⁸, at least 1×10⁹ or more total CD4+CD8− SP T cells.

In some embodiments of any of the aspects, the method further comprises isolating the population of CD4+CD8− SP T cells.

In some embodiments of any of the aspects, the CD4+CD8− SP T cells are CD3+ and TCRαβ+.

In some embodiments of any of the aspects, the CD4+CD8− SP T cells are CD62L+, CD5+, CCR7+, MHC I+, CD69+, and/or CD25+.

In some embodiments of any of the aspects, the CD4+CD8− SP T cells express T-helper-inducing POZ-Kruppel-like factor (ThPOK) and/or T cell receptor alpha chain constant (TRAC).

In some embodiments of any of the aspects, the CD4+CD8− SP T cells are capable upon stimulation of secreting at least one of the following: interferon-γ (IFNγ), tumor necrosis factor-α (TNFα), interleukin-2 (IL-2), interleukin 4 (IL-4), interleukin 5 (IL-5), interleukin 6 (IL-6), interleukin 8 (IL-8), interleukin 9 (IL-9), interleukin 10 (IL-10), interleukin 13 (IL-13), interleukin 17a (IL-17a), interleukin 17f (IL-17f), interleukin 22 (IL-22), amphiregulin (AREG), granzyme B, macrophage inflammatory protein-1 alpha (MIP1α), and/or macrophage inflammatory protein-1 beta (MIP1β), or any combination thereof.

In some embodiments of any of the aspects, at least 90% of the CD4+CD8− SP T cells in the population are capable upon stimulation of secreting IFNγ and TNFα.

In some embodiments of any of the aspects, the method further comprises, prior to culturing the population of DP T progenitor cells, a step of: culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells.

In some embodiments of any of the aspects, the Notch ligand comprises Delta-like ligand 1 (DLL1) and/or Delta-like ligand 4 (DLL4).

In some embodiments of any of the aspects, the CD34+ HSPCs are cultured for at least 28 days.

In some embodiments of any of the aspects, the method further comprises, prior to culturing the population of CD34+ HSPCs, a step of: culturing a population of pluripotent stem cells in a medium comprising: bone morphogenetic protein-4 (BMP-4); vascular endothelial growth factor (VEGF); and a glycogen synthase kinase-3β inhibitor, until the population of pluripotent stem cells differentiates into the population of CD34+ HSPCs.

In some embodiments of any of the aspects, the population of pluripotent stem cells comprises induced pluripotent stem cells (iPSCs).

In some embodiments of any of the aspects, the population of pluripotent stem cells is cultured for at least 12 days.

In some embodiments of any of the aspects, the method further comprises isolating the CD34+ HSPCs.

In some embodiments of any of the aspects, the method further comprises, prior to culturing the population of pluripotent stem cells, genetically modifying the population of pluripotent stem cells to express a polypeptide exogenous to the pluripotent stem cell.

In some embodiments of any of the aspects, the polypeptide comprises a chimeric antigen receptor (CAR).

In some embodiments of any of the aspects, a nucleic acid encoding the polypeptide is integrated at the T-cell receptor alpha constant locus (TRAC) locus or the adeno-associated virus integration site 1 (AAVS1) locus of the genome of the pluripotent stem cell.

In some embodiments of any of the aspects, the antigen-binding domain of the CAR comprises a single-chain variable fragment that specifically binds CD19.

In some embodiments of any of the aspects, the intracellular signaling domain of the CAR comprises a CD3ζ signaling module.

In some embodiments of any of the aspects, the CAR further comprises a costimulatory signaling domain of CD28 and/or 4-1BB.

In some embodiments of any of the aspects, the method further comprises differentiating the population of CD4+CD8− SP T cells into a population of regulatory T cells by culturing in a medium comprising transforming growth factor-beta (TGF-β), interleukin-2 (IL-2), retinoic acid, and rapamycin.

In one aspect, described herein is a population of CD4+CD8− SP T cells produced using a method as described herein.

In one aspect, described herein is a population of regulatory T cells produced using a method as described herein.

In one aspect, described herein is a composition comprising a population as described herein.

In one aspect, described herein is a pharmaceutical composition comprising a population as described herein and a pharmaceutically acceptable carrier.

In one aspect, described herein is a method of adoptive cell transfer comprising administering an effective amount of a population as described herein, or a composition as described herein, or a pharmaceutical composition as described herein, to a subject in need thereof.

In some embodiments of any of the aspects, the population of cells is autologous to the subject.

In some embodiments of any of the aspects, the population of cells is allogeneic to the subject.

In one aspect, described herein is a method of treating an autoimmune disease comprising administering an effective amount of a population as described herein, or a composition as described herein, or a pharmaceutical composition as described herein, to a subject in need thereof.

In one aspect, described herein is a method of treating cancer comprising administering an effective amount of a population as described herein, or a composition as described herein, or a pharmaceutical composition as described herein, to a subject in need thereof.

In one aspect, described herein is a method of treating an infectious disease comprising administering an effective amount of a population as described herein, or a composition as described herein, or a pharmaceutical composition as described herein, to a subject in need thereof.

In one aspect, described herein is use of a population as described herein in an in vitro drug screening assay.

In one aspect, described herein is use of a population as described herein in an immunological assay.

The foregoing and other aspects of this disclosure will become apparent from the detailed description and examples that follow, as well as from the appended claims.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1G: Analysis of cells from throughout the process of generating and maintaining iCD4+ T cells. (FIG. 1A) Schematic of iT differentiation to CD4+ cells. (FIG. 1B) Representative flow cytometry of key developmental timepoints and metrics of DP T cells. iPSCs were plated at day-2 and cultured for 12 days in HSPC differentiation conditions. The resultant CD34+ HSPCs were purified using magnetic beads. Day 12 HSPCs were moved to Notch-stimulatory lymphoid coating and cultured with STEMCELL TECHNOLOGIES lymphoid expansion media until day 26, where cells were assayed for CD7 expression. Day 26 cells were replated on fresh Notch stimulatory coating and cultured with STEMCELL TECHNOLOGIES lymphoid maturation media to day 40, where cells are assayed for TCR, CD3, CD4 and CD8 expression. (N=3) (FIG. 1C) Representative flow cytometry of D54 T cells, with parent gating in FIG. 8A. Day 40 iPSC DP T cells were moved to RETRONECTIN coating and treated with anti-CD3 antibody (OKT3, 5 μg/ml), half media changes were performed every 3 or 4 days. (N=3) (FIG. 1D) Representative flow cytometry of D68 T cells post expansion. Day 54 iCD4+ T cells were moved to fresh RETRONECTIN coated wells and treated with media supplemented with anti-CD3/CD28 and 200 U/ml IL-2. (N=3) (FIG. 1E) Left bar graph: Percentage of CD4 and CD8 positive T cells from iCD4+ T cell differentiation by flow cytometry at day 40, 54 and 68, N=3, analyzed by 2-way ANOVA followed by a Tukey's multiple comparison test. *P<0.05: **P<0.01: ***P<0.001: ****P<0.0001; the left-right order of the bars in each group is the same as the left-right order of the legend. Right line graph: Number of cells, starting with initial iPSC seeding: dotted line indicates timepoints of stimulation of T cells. (FIG. 1F) Representative flow cytometry of T cells from long-term culture in X-VIVO 15 media supplemented with 200 U/ml IL-2. N=3, parent gating for each plot as indicated. (FIG. 1G) Number of doublings of iCD4+ T cells after stimulation with anti-CD3/CD28 (N=3).

FIGS. 2A-2D: Maturation marker flow cytometry of multiple time points of CD4 specification (day 40 and day 54) and expansion (day 68 and day 75) of iT cells. The populations shown are gated on Live/Single/CD45+/CD14−/CD16−/CD56−/CD335−/CD337−/CD3+/TCRαβ+ cells, with the exception of Day 54, which is gated on Live/Single/CD45+/CD14−/CD16−/CD56−/CD335−/CD337− cells due to poor CD3 and TCRαβ expression. Parent gating shown in FIG. 8B, representative of N=3 differentiations, iPSC DP T cells were specified to iCD4+ T cells using the differentiation outlined in FIG. 1A and analyzed for expression of CD5 (FIG. 2A). CD62L and CCR7 (FIG. 2B). CD69 and MHC I (FIG. 2C), and CD25 (FIG. 2D).

FIGS. 3A-3D: CyTOF analysis of iCD4+ T cells. (FIG. 3A) Expression profile of CD3, CD56, CD4 and CD8 in the presence and absence of overnight PMA/ionomycin stimulation. (FIG. 3B) opt-SNE projections of stimulated iCD4+ T cells overlaid with color-coded PHENOGRAPH clusters 1 through 16, as well as with protein expression heatmaps of intracellular analytes (FIG. 3C) Bivariate plots of stimulation-specific upregulation of cytokines and other functional proteins in iCD4+ T cells. (FIG. 3D) Heatmap showing column-normalized expression of each marker in PHENOGRAPH clusters. The first 6 rows show subsets derived from control PBMC, while remaining 16 rows correspond to clusters outlined in FIG. 3B.

FIG. 4: Analysis of cytokine secretion by iCD4+ T cells. Concentration of cytokines (pg/ml) in the supernatant of media after six hours of PMA/Ionomycin stimulation. iCD4+ T cells and CD4+ T cells purified from PBMC by magnetic selection were plated in X-VIVO 15 media at 1*10\hat{ }6 cells/ml. The unstimulated group was rested, while all other groups were treated with PMA (25 ng/ml) and ionomycin (1 μg/ml) at 37 degrees C. for 6 hours. Supernatant was collected and analyzed in technical duplicate by 12-Plex LEGENDplex™ HU Th Cytokine Panel (BIOLEGEND) on a CYTEK AURORA. Concentration of cytokines were determined using a standard curve by LEGENDplex™ software (BIOLEGEND). Dotted line indicates the upper limit of detection for the cytokine. N=3, 3, 5 and 2 for unstimulated D68 iT cells. PBMC T cells. D68 iT cells, and D140+iT cells respectively. Data shown are group means+/−SD analyzed by one-way ANOVA with Tukey's multiple comparison test. *P<0.05: **P<0.01: ***P<0.001: ****P<0.0001. The left-right order of the bars in each graph is the same as the left-right order of the legend.

FIGS. 5A-5E: scRNA sequencing reveals characteristic CD4 transcripts in iCD4+ T cells. (FIG. 5A) Schematic of scRNAseq experiment. Live cells were purified from iCD4+ T cells cultures at day 71 and 103 of culture by magnetic depletion of debris. CD3+ T cells were isolated from PBMC by magnetic selection. (FIG. 5B) UMAP projections overlaid with coded sample identity (left) derived from Louvain clustering at resolution 0.15 where each shading represents annotated clusters. Characteristic marker genes used for annotation indicated in FIG. 11C. (FIG. 5C) UMAP shaded by gene expression for key T cell identity, activation and functional genes. (FIG. 5D) Bubble plot displaying expression of top 25 DEGs for CD4+ (top 25 rows) and CD8+ (remaining rows) T cells from literature in each cluster previously defined in FIG. 5B, genes listed in order of highest to lowest significance from the source DEG analysis. (FIG. 5E) Key T cell and CD4+ specific marker and transcription factor gene expression data was imputed using ALRA and displayed using a violin plot for each original identity.

FIG. 6: Anti-CD3 treatment alone improves viability of iPSC derived CD4+ T cells. Representative viability and CD4/8 flow cytometry of iT cells generated 14 days after DP iT cells are stimulated with anti-CD3/CD28 (25 ul/ml IMMUNOCULT, STEMCELL) or anti-CD3 (5 μg/ml OKT3).

FIGS. 7A-7C. CD4 specification in additional iPSC lines with and without inducible NICD1. (FIG. 7A) Flow cytometry of WT BU1 background iT cells after CD4 specification and 1 round of expansion. (FIG. 7B) Flow cytometry of BU1N1, BU1, BU2N1 and BU7 background iT cells after CD4 specification and 1 round of expansion. (FIG. 7C) qPCR analysis of ThPOK transcription over 1 differentiation of BU1N1 cells at day 40, 54 and 68, relative to PBMC T cells using ACTb as a housekeeping gene in technical triplicate. Mean±SEM.

FIGS. 8A-8B: Gating strategy for flow cytometry analysis. (FIG. 8A) Gating strategy for iT cells in FIG. 1. (FIG. 8B) Gating strategy for iT cells in FIG. 2.

FIGS. 9A-9B: TCRb sequencing of iCD4 T cells. (FIG. 9A) Abundance plots of TRBV genes and heatmaps of TRBV and TRBJ gene usage in both iCD4 cells at day 68 and T cells purified from PBMC. RNA was extracted from snap-frozen cell pellets of one million iCD4 T cells at day 68 of culture and sequenced. (FIG. 9B) Unique gene frequency plot of TRBV genes in iCD4 T cell sample.

FIGS. 10A-10B: Expression of additional proteins by CyTOF. (FIG. 10A) iCD4 comparison to PBMC derived T cells with and without overnight PMA/ionomycin stimulation. (FIG. 10AB) optSNE map of stimulated iCD4+ T cells colored by expression of additional markers.

FIGS. 11A-11D: Additional genes revealed in scRNAseq for annotation and CD4 identity of iT cells. (FIG. 11A) UMAP showing only PBMC cells. (FIG. 11B) UMAP shaded by expression of naïve CD4 genes. (FIG. 11C) UMAP shaded by expression of markers for proliferating cells, NKT cells, myeloid cells, B cells and MAIT cells. (FIG. 11D) Violin plot of imputed data for key clusters showing key CD4 T cell gene expression.

FIGS. 12A-12C Module scores by cluster from scRNAseq experiment. Module score for CD4+ T cells (FIG. 12A), CD8+ TRAV 1-2-T cells (FIG. 12B), and myeloid cells (FIG. 12C), derived from the top 100 DEGs in these populations from a large scale single cell RNA sequencing study of human PBMC (see e.g., Terekhova et al., 2024) plotted by cluster.

FIGS. 13A-13B: Analysis of cytokine secretion by iCD4+ T cells. (FIG. 13A) Concentration of cytokines in the supernatant of media after PMA/Ionomycin stimulation of T cells at 1*10\hat{ }6 cells/mL in picograms/mL. Dotted line indicates the upper limit of detection for the cytokine. N varies from 1 to 3 per group. The left-right order of the bars in each graph is the same as the left-right order of the legend. (FIG. 13B) Flow cytometry of PBMC and Day 140+ iT cells for CD4 and intracellular interferon γ, pre-gated on live cells.

FIG. 14: Single cell RNA sequencing UMAP of PBMC. D71 and D103 iT cells showing selected genes.

### DETAILED DESCRIPTION

Over time, the directed differentiation of human induced pluripotent stem cells (iPSCs) into T lymphocytes has opened the door for generating uniform, patient-specific immune effectors in vitro. Early protocols have focused on patterning mesodermal precursors and hemogenic endothelium through precise activation of developmental pathways, often via small-molecule inhibitors, followed by sequential exposure to defined growth factors and signaling ligands. Stromal co-cultures expressing Notch ligands or immobilized fusion proteins have been employed to drive lineage specification toward double-positive thymocyte progenitors. However, most resulting populations resolve into single-positive cells, and only a minor fraction attains a helper (CD4+) phenotype, reflecting persistent challenges in directing iPSC-derived progenitors toward this specific lineage.

In vivo, double-positive thymocyte maturation depends on a finely tuned interplay of Notch, TCR, co-receptor engagement, and cytokine support within the thymic microenvironment. In response, in vitro systems have aimed to replicate these cues using three-dimensional organoid cultures, artificial thymic organoids, or feeder-free matrices supplemented with tailored cytokine cocktails. However, the mechanical forces required for effective receptor triggering, the precise timing and intensity of ligand presentation, and the spatial arrangement of co-stimulatory signals present significant challenges. As a result, coordinating interleukin signaling and the timely withdrawal of stromal factors remains challenging in standard culture formats, and achieving reliable commitment to the CD4+ helper lineage has proven difficult in scalable differentiation workflows.

Helper T cells play a central role in adaptive immunity, providing cytokine support and enhancing the survival and function of cytotoxic subsets. In adoptive cell therapies, the balance between helper and cytotoxic populations has a significant impact on persistence, potency, and safety, particularly in engineered T-cell approaches targeting cancer and chronic viral infections. Autologous T-cell harvesting faces challenges due to donor variability and lengthy manufacturing timelines, whereas allogeneic sources involve immunogenicity risks. As a result, the generation of functional helper T cells from iPSCs represents a promising alternative, though it requires differentiation strategies that effectively combine hematopoietic progenitor specification with specialized maturation steps to produce naïve and effector helper phenotypes. To date, most methods focus on either early progenitor induction or cytotoxic T-cell maturation, leaving a gap in protocols capable of producing bona fide helper T cells.

Human iPSCs offer a renewable platform for engineering immune cells with precise genetic modifications, such as chimeric antigen receptors, safety switches, and immunogenicity-reducing edits. A robust, scalable approach for deriving functional helper T cells from iPSCs would permit off-the-shelf cell-therapy products with consistent quality, streamlined production, and broad applicability across oncology, autoimmunity, and infectious diseases. Accordingly, there remains a pressing need for refined differentiation strategies that reliably generate single-positive helper T cells at clinically relevant scales.

The present disclosure pertains to methods and compositions for generating CD4⁺CD8⁻ single-positive T cells from induced pluripotent stem cells (iPSCs) and their therapeutic applications. The described approach is positioned within the broader field of immunology, regenerative medicine, and cell therapy, with a focus on the development of scalable, feeder-free protocols for producing functional T cell populations. In some embodiments, the methods described herein do not comprise use of feeder cells (e.g., bone marrow stromal cells that express a Notch ligand, e.g., OP9-Delta-like 1 (DL1) cells, OP9-Delta-like 4 (DL4) cells). These methods address longstanding challenges in guiding iPSC-derived progenitors toward the CD4⁺ helper T cell lineage, which plays an important role in adaptive immunity and holds significant promise in adoptive cell therapies for cancer, autoimmune diseases, and other conditions.

The embodiments and examples described herein are provided for illustrative purposes and are not intended to limit the scope of the described subject matter. Certain details, such as standard laboratory techniques or widely recognized principles in the field, may be omitted for clarity and conciseness. Additionally, various modifications, substitutions, or rearrangements of the described methods and compositions may be made without departing from the spirit and scope of the subject matter, as defined by the appended claims.

CD4⁺CD8⁻ Single-Positive T Cells

CD4⁺ single-positive T cells, commonly referred to as helper T cells, are a subset of the adaptive immune system that coordinates immune responses by activating and regulating other immune cells through cytokine secretion and cell-to-cell interactions. CD4⁺ single-positive T cells are identified by the expression of specific surface markers and transcription factors that define their identity and functionality. Markers used to identify CD4⁺ helper T cells include CD3, a component of the T cell receptor (TCR) complex; TCRαβ, which mediates antigen recognition; and CD4, the defining co-receptor that interacts with molecules of the histocompatibility complex class II (MHC II). Additional markers include CD62L and CCR7, which are associated with lymph node homing: CD5, a regulator of TCR signaling; and CD25, the interleukin-2 receptor alpha chain indicative of activation. Mature helper T cells also express transcription factors such as ThPOK (ZBTB7B), which is necessary for CD4 lineage commitment, and surface molecules like CD69 and CD45RO, which are indicative of activation and memory states, respectively. These markers collectively support the accurate identification and functional characterization of CD4⁺ helper T cells in both research and clinical settings.

The generation of functional CD4⁺ T cells from induced pluripotent stem cells (iPSCs) has long been a significant challenge in the field of immunology and regenerative medicine. While existing protocols have successfully directed iPSCs toward hematopoietic progenitors and even CD8⁺ cytotoxic T cells, the reliable and scalable production of CD4⁺ helper T cells has remained elusive. Conventional approaches often rely on Notch ligand-mediated signaling and anti-CD28 co-stimulation to drive T cell differentiation. However, these methods frequently result in populations dominated by CD8⁺ T cells, with only a minor fraction of cells adopting the CD4⁺ helper phenotype. This imbalance reflects the difficulty in recapitulating the precise signaling and environmental cues required for CD4⁺ lineage commitment. Furthermore, the dependency on feeder cells, complex co-culture systems, and exogenous Notch ligands introduces significant variability, scalability issues, and manufacturing inefficiencies, limiting the clinical and commercial viability of such methods.

The present method addresses these limitations by providing a feeder-free and scalable approach for generating CD4⁺CD8⁻ single-positive (SP) T cells from iPSCs. Unlike conventional protocols, the disclosed approach eliminates Notch ligand-mediated signaling and CD28 co-stimulation using an anti-CD28 agent during the double-positive (DP) to single-positive (SP) transition. Instead, the method utilizes a defined culture system comprising interleukin-7 (IL-7) and an anti-CD3 agent to guide differentiation toward the CD4⁺ lineage. By systematically withdrawing Notch ligands and anti-CD28 agents, the approach overcomes the apoptotic and lineage-skewing effects observed in prior methods, permitting robust CD4⁺ T cell specification. The resulting CD4⁺CD8⁻ SP T cells exhibit functional competency, including the ability to proliferate, express surface markers and transcription factors relevant to their function, and secrete a diverse array of cytokines upon stimulation.

This disclosed methodology not only simplifies the differentiation process but also improves reproducibility and scalability, making the approach well-suited for clinical and industrial applications. By removing the reliance on complex co-culture systems and feeder cells, the described method optimizes cell manufacturing workflows, reduces costs, and facilitates the production of high-quality CD4 T cells at scales relevant to clinical needs. Furthermore, the platform is compatible with genetic modifications, such as the incorporation of chimeric antigen receptors (CARs), thereby expanding its applicability in adoptive cell therapies for cancer, autoimmune diseases, and other conditions. Overall, the disclosed approach represents an advancement in the field, addressing persistent challenges and creating new possibilities for the therapeutic use of iPSC-derived CD4 T cells.

In one embodiment, the medium used for culturing the DP T progenitor cells comprises interleukin-7 (IL-7) at a concentration of about 10 ng/ml and an anti-CD3 antibody, such as clone OKT3, at a concentration of 5 μg/mL, to promote differentiation into CD4+CD8− single-positive (SP) T cells. In another embodiment, the population of CD4+CD8+ double-positive (DP) T progenitor cells is cultured on a substrate coated with fibronectin or a recombinant embodiment thereof, such as RetroNectin®, at a concentration of at least 10 μg/mL, to support cell adhesion and differentiation. In yet another embodiment, the culturing process is carried out for at least 14 days to ensure sufficient differentiation of the DP T progenitor cells into the desired SP T cell population. Additionally, variations in the culture conditions may include the use of alternative recombinant fibronectin embodiments or substrates that provide similar support for cell survival and differentiation. Furthermore, the absence of Notch ligands and anti-CD28 agents during the culturing process is significant, and alternative methods to ensure the removal of these factors, such as specific media formulations or coatings, may be employed.

In some embodiments, the method of differentiating a population of DP T progenitor cells into a population of CD4+CD8− SP T cells does not comprise use of a Notch ligand. In some embodiments, the method of differentiating a population of DP T progenitor cells into a population of CD4+CD8− SP T cells does not comprise use of an anti-CD28 agent. In some embodiments, the method of differentiating a population of DP T progenitor cells into a population of CD4+CD8− SP T cells does not comprise use of a Notch ligand and does not comprise use of an anti-CD28 agent.

In one aspect, described herein is a method of producing a population of CD4+CD8− single-positive (SP) T cells, the method comprising: culturing a population of CD4+CD8+ double positive (DP) T progenitor cells in the absence of: Notch ligand and an anti-CD28 agent, and in a medium comprising interleukin-7 (IL-7) and an anti-CD3 agent for a sufficient amount of time, e.g., until the population of DP T progenitor cells differentiates into the population of CD4+CD8− SP T cells. The population of DP T progenitor cells can be cultured for at least 7 days, at least 10 days, at least 14 days, at least 21 days, or for a period within a range such as 7-10 days, 10-14 days, or 14-21 days.

In some embodiments, the CD4+CD8− SP T cells are further cultured to increase proliferation of the cells. In one embodiment, the population of CD4+CD8− SP T cells is cultured in a medium comprising: an anti-CD3 agent, an anti-CD28 agent, and interleukin-2 (IL-2), e.g., for a sufficient amount of time to increase proliferation of the cells and/or the cell number of the population. For example, the population of CD4+CD8− SP T cells can undergo at least 2, at least 3, at least 4, at least 5, or at least 6 population doublings. The population of CD4+CD8− SP T cells can be cultured for at least 14 days to at most 140 days, at least 14 days, at least 28 days, at least 35 days, at least 70 days, at least 100 days, or at least 140 days, or for a period within a range such as 14-28 days, 28-35 days, 35-70 days, 70-100 days, or 100-140 days.

In one aspect, described herein is a method of producing a population of CD4+CD8− single-positive (SP) T cells, the method comprising: (a) culturing a population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells; and (b) culturing the resultant population of CD4+CD8− SP T cells in a medium comprising: the anti-CD3 agent, the anti-CD28 agent, and interleukin-2 (IL-2), e.g., to increase proliferation of the population of CD4+CD8− SP T cells.

In some embodiments, the CD4+CD8− SP T cells are cultured in Stemspan™ SFEM II medium supplemented with Lymphoid Progenitor Maturation Supplement (STEMCELL TECHNOLOGIES) and Primocin™, which further comprises IL-2, the anti-CD3 agent, and the anti-CD28 agent, as described herein (see e.g., Table 1). In some embodiments, the CD4+CD8− SP T cells are cultured in X-Vivo® 15 media (LONZA), which further comprises IL-2 (see e.g., Table 1). X-Vivo® 15 media (LONZA) is a serum-free, xenofree hematopoietic cell medium, with L-Glutamine, gentamicin, phenol red, human albumin. recombinant human insulin, and/or pasteurized human transferrin.

In some embodiments, the CD4+CD8− SP T cells are isolated prior to further steps, e.g., using CD4-specific Magnetic-Activated Cell Sorting (MACS) or CD4-specific Fluorescence-Activated Cell Sorting (FACS).

Accordingly, in one aspect, described herein is a population of CD4+CD8− SP T cells produced using the methods as described herein. In some embodiments, the population of CD4+CD8− SP T cells produced by a method as described herein comprises at least 40%, at least 41%, at least 42%, at least 45%, at least 48%, at least 50%, at least 55%, at least 60%, at least 65%, at least 70%, at least 75%, at least 80%, at least 85%, at least 90%, at least 95%, or more CD4+CD8− SP T cells. In further embodiments, the population comprises a range of CD4+CD8− SP T cells, such as 40%-50%, 50%-60%, 60%-70%, 70%-80%, 80%-90%, 90%-100%, or 80%-100% CD4+CD8− SP T cells.

In some embodiments, the population of CD4+CD8− SP T cells is produced by a method as described herein at a large scale. For example, therapeutic applications can include more than 1×10⁹ (1 billion) T cells for infusion into a subject. In some embodiments, a population comprising at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, at least 2×10⁹, at least 3×10⁹, at least 4×10⁹, at least 5×10⁹, at least 6×10⁹, at least 7×10⁹, at least 8×10⁹, at least 9×10⁹, at least 1×10¹⁰, at least 2×10¹⁰, at least 3×10¹⁰, at least 4×10¹⁰, at least 5×10¹⁰, at least 6×10¹⁰, at least 7×10¹⁰, at least 8×10¹⁰, at least 9×10¹⁰, at least 1×10¹¹, at least 2×10¹¹, at least 3×10¹¹, at least 4×10¹¹, at least 5×10¹¹, at least 6×10¹¹, at least 7×10¹¹, at least 8×10¹¹, at least 9×10¹¹, at least 1×10¹², 1×10⁷ to 1×10⁸, 1×10⁸ to 1×10⁹, 1×10⁹ to 1×10¹⁰, 1×10¹⁰ to 1×10¹¹, or 1×10¹¹ to 1×10¹², or more total CD4+CD8− SP T cells can be produced using the methods as described herein, e.g., using a bioreactor.

In some embodiments, the CD4+CD8− SP T cells produced by a method as described herein are CD3+ and TCRαβ+. In some embodiments, the CD4+CD8− SP T cells produced by a method as described herein are CD62L+, CD5+, CCR7+, MHC I+, CD69+, and/or CD25+.

In some embodiments, the CD4+CD8− SP T cells produced by a method as described herein express T-helper-inducing POZ-Kruppel-like factor (ThPOK) and/or T cell receptor alpha chain constant (TRAC).

In some embodiments, the CD4+CD8− SP T cells produced by a method as described herein are capable (e.g., upon stimulation, e.g., by ionomycin and PMA, or by an antigen-presenting cell) of cytokine secretion. Non-limiting examples of cytokines and other agents secreted by the stimulated CD4+CD8− SP T cells include: interferon-γ (IFNγ), tumor necrosis factor-α (TNFα), interleukin-2 (IL-2), interleukin 4 (IL-4), interleukin 5 (IL-5), interleukin 6 (IL-6), interleukin 8 (IL-8), interleukin 9 (IL-9), interleukin 10 (IL-10), interleukin 13 (IL-13), interleukin 17a (IL-17a), interleukin 17f (IL-17f), interleukin 22 (IL-22), amphiregulin (AREG), granzyme B, macrophage inflammatory protein-1 alpha (MIP1α), and/or macrophage inflammatory protein-1 beta (MIP1β), or any combination thereof.

In some embodiments, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the CD4+CD8− SP T cells in the population produced by a method as described herein are capable upon stimulation of secreting IFNγ and TNFα.

In some embodiments, the population of CD4+CD8− SP T cells following stimulation are effector memory CD4+CD8− SP T cells. Such effector memory CD4+CD8− SP T cells can be capable of surviving at least 30 days, at least 40 days, at least 70 days, at least 100 days, at least 140 days, at least 180 days, at least 200 days, or at least 1 year following stimulation, or for a period within a range such as 30-70 days, 70-100 days, 100-180 days, 180-365 days, or 1-2 years.

In some embodiments, the CD4+CD8− SP T cells produced by a method as described herein are capable of helper T cell functions, including but not limited to activation of dendritic cells, e.g., maturation and/or licensing: activation of B cells, e.g., to produce antibodies: activation of cytotoxic (CD4−CD8+ SP) T cells, e.g., to kill cells; and/or activation of macrophages, e.g., to engulf pathogens.

In some embodiments, the CD4+CD8− SP T cells are further differentiated or polarized into CD4 T cell subtypes, including but not limited to, regulatory T (Treg), Th1, Th2, Th3, Th9, Th17, Th22, T follicular helper (Tfh), or granulocyte-macrophage-colony-stimulating factor (GM-CSF)-producing T helper (ThGM) cells, using methods as known in the art. As a non-limiting example, the method can further comprise differentiating the population of CD4+CD8− SP T cells into a population of regulatory T cells by culturing in a medium comprising transforming growth factor-beta (TGF-β), interleukin-2 (IL-2), retinoic acid, and rapamycin. Accordingly; in one aspect, described herein is a population of regulatory T cells produced using the methods as described herein. Non-limiting examples of markers expressed by such Tregs include CD4, CD25, FOXP3, and/or CTLA-4.

### Anti-CD3 Agents

CD3 is a multi-subunit protein complex that is a component of the T cell receptor (TCR) complex on the surface of T lymphocytes. Components of the CD3 complex include CD3ε, CD3γ, CD3δ, and CD3ζ. CD3 plays a role in T cell development, activation, and differentiation by transmitting activation signals from the TCR upon antigen recognition. Engagement of CD3 leads to intracellular signaling cascades that drive T cell maturation and functional responses. Specifically, the CD3 intracellular signaling cascade begins when T cells encounter an antigen via the T cell receptor (TCR). Upon antigen recognition, the CD3 complex, associated with the TCR, transmits signals through its immunoreceptor tyrosine-based activation motifs (ITAMs). The intracellular tails of the CD3γ, CD3ε, and CD3δ components each comprise an ITAM. The intracellular tail of CD3ζ comprises 3 ITAM motifs. These ITAMs become phosphorylated by Src-family kinases (e.g., LCK, FYN), triggering a cascade involving proteins such as LAT, SLP-76, and ZAP-70. This leads to downstream pathways, including calcium influx and activation of the Ras/MAPK and PKC pathways, which induces the activation of transcription factors such as NF-κB, NFAT, and AP-1, resulting in T cell proliferation and activation. In the context of T cell differentiation protocols described herein, stimulation with anti-CD3 agents mimics physiological TCR signaling, thereby promoting the specification and maturation of T cell populations in vitro.

An exemplary sequence for the human CD3 epsilon (CD3ε) subunit, a component of the CD3 complex, is as follows (UniProtKB/Swiss-Prot: P07766), extracellular domain bolded (corresponding to amino acids (aa) 23-126; aa 1-22 correspond to signal peptide; aa 127-152 correspond to helical transmembrane domain; aa 153-207 correspond to intracellular domain):

(SEQ ID NO: 1)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

An exemplary sequence for the human CD3 gamma (CD3γ) subunit, a component of the CD3 complex, is as follows (UniProtKB/Swiss-Prot: P09693), extracellular domain bolded (corresponding to amino acids (aa) 23-116; aa 1-22 correspond to signal peptide; aa 117-137 correspond to helical transmembrane domain: aa 138-182 correspond to intracellular domain):

(SEQ ID NO: 2)
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN

An exemplary sequence for the human CD3 delta (CD3δ) subunit, a component of the CD3 complex, is as follows (UniProtKB/Swiss-Prot: P04234), extracellular domain bolded (corresponding to amino acids (aa) 22-105; aa 1-21 correspond to signal peptide; aa 106-126 correspond to helical transmembrane domain; aa 127-171 correspond to intracellular domain):

(SEQ ID NO: 3)
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK

An exemplary sequence for the human CD3 zeta (CD3) subunit, a component of the CD3 complex, is as follows (UniProtKB/Swiss-Prot: P20963), extracellular domain bolded (corresponding to amino acids (aa) 22-30; aa 1-21 correspond to signal peptide; aa 31-51 correspond to helical transmembrane domain: aa 52-164 correspond to intracellular domain):

(SEQ ID NO: 4)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

An anti-CD3 agent can be used in the methods described herein. The term “anti-CD3 agent” refers to an agent that is capable of specifically binding to CD3 and activating CD3 intracellular signalling. The anti-CD3 agent can also be referred to as a CD3 agonist. Non-limiting examples of anti-CD3 agents include polypeptides (e.g., antibodies), nucleic acids (e.g., aptamers), and small molecules (see e.g., Deng et al. “Back on the scene: Advances and challenges in CD3-related drugs in tumor therapy.” Drug discovery today vol. 27,8 (2022): 2199-2208: Menon et al. “Modulating T Cell Responses by Targeting CD3.” Cancers (Basel). 2023 Feb. 13; 15 (4): 1189). In some embodiments, the anti-CD3 agent comprises an antibody, or an antigen-binding domain thereof, or an aptamer. In some embodiments, the anti-CD3 agent specifically binds to human CD3. In some embodiments, the anti-CD3 agent specifically binds to mammalian (e.g., human, murine, non-human primate, pig, and/or rat) CD3.

In some embodiments, the anti-CD3 agent specifically binds to the CD3ε subunit (see e.g., SEQ ID NO: 1), e.g., the extracellular domain of the CD3ε subunit. In some embodiments, the anti-CD3 agent specifically binds to the CD3γ subunit (see e.g., SEQ ID NO: 2), e.g., the extracellular domain of the CD3γ subunit. In some embodiments, the anti-CD3 agent specifically binds to the CD3δ subunit (see e.g., SEQ ID NO: 3), e.g., the extracellular domain of the CD3δ subunit. In some embodiments, the anti-CD3 agent specifically binds to the CD3ζ subunit (see e.g., SEQ ID NO: 4), e.g., the extracellular domain of the CD3ζ subunit.

In some embodiments, the anti-CD3 agent is not bound to a substrate (e.g., a cell culture dish, bead, etc.), which can decrease its clustering of CD3 and/or decrease its activation of CD3 intracellular signalling. In some embodiments, the anti-CD3 agent is free in solution (e.g., in the cell culture medium). In some embodiments, the anti-CD3 agent is in a tetrameric complex, which is a soluble, four-part antibody complex or “tetramer”.

In some embodiments, the anti-CD3 agent comprises an anti-CD3 antibody or an antigen binding domain thereof. Targeting domains, e.g., anti-CD3 antibodies, antibody reagents, nanobodies, and polypeptides specific for a selected target or substrate are readily available in the art. For example, antibodies for a selected target or substrate (e.g., CD3) can be identified by one of skill in the art using The Antibody Registry (available on the world wide web at antibodyregistry.org); Antibodypedia (available on the world wide web at antibodypedia.com); the Validated Antibody Database (available on the world wide web at labome.com/index.html); or ABCD (available on the world wide web at web.expasy.org/abcd/); or commercial sources such as AbCam, Sigma, ThermoFisher, and BioLegend. One of skill in the art can readily select a targeting domain depending on the target or substrate to be bound. In some embodiments, the anti-CD3 antibody is selected from Table 4. In some embodiments, the anti-CD3 antibody is selected from OKT3, Otelixizumab, Teplizumab, HIT3a, UCHT1, 17A2, 145-2C11, or Foralumab. In some embodiments, the anti-CD3 antibody is OKT3. In some embodiments, the anti-CD3 antibody is UCHT1.

TABLE 4
Exemplary anti-CD3 antibodies

| | | catalog/ | |
|---|---|---|---|
| | | accession | |
| mAb name | source | number | links and references |
| Muromonab | CELL | 92511 | US patents U.S. Pat. No. 7,994,289 B2, |
| (OKT3) | SIGNALING | | U.S. Pat. No. 11,913,024 B2; |
| | TECHNOLOGY | | precision.fda.gov/ginas/app/ui/substances/3d466d5d- |
| | | | dc5c-4615-89f0-f755a1dcb631; |
| | | | cellsignal.com/products/primary- |
| | | | antibodies/human-cd3e-activating-okt3-mouse- |
| | | | mab-low-endotoxin-azide-free/92511 |
| CD3 | THERMOFISHER | 16-0037- | thermofisher.com/antibody/product/CD3- |
| Monoclonal | | 81 | Antibody-clone-OKT3-Monoclonal/16-0037-81; |
| Antibody | | | Kuhn, Chantal, and Howard L Weiner. |
| (OKT3), | | | “Therapeutic anti-CD3 monoclonal antibodies: |
| Functional | | | from bench to bedside.” Immunotherapy vol. 8.8 |
| Grade, | | | (2016): 889-906. doi: 10.2217/imt-2016-0049 |
| eBioscience ™ | | | |
| Otelixizumab | SELLECKCHEM | A3004 | precision.fda.gov/ginas/app/ui/substances/6cecf29e- |
| or TRX4 | | | 1340-49b4-a066-ff651c9da50a |
| Teplizumab | THERMOFISHER | MA5- | precision.fda.gov/ginas/app/ui/substances/7cf8c6e9- |
| or Tzield | | 59029 | 6dcf-45d4-8ee3-5a54e67d1dd2 |
| CD3 | THERMOFISHER | 16-0039- | thermofisher.com/antibody/product/CD3- |
| Monoclonal | | 81 | Antibody-clone-HIT3a-Monoclonal/16-0039-81 |
| Antibody | | | |
| (HIT3a), | | | |
| Functional | | | |
| Grade, | | | |
| eBioscience ™ | | | |
| Human CD3 | R&D SYSTEMS | MAB100 | rndsystems.com/products/human-cd3epsilon- |
| epsilon | | | antibody-ucht1_mab100; |
| Antibody | | | Zumrut, Hasan E. et al., Integrating Ligand- |
| (UCHT1) | | | Receptor Interactions and In Vitro Evolution for |
| | | | Streamlined Discovery of Artificial Nucleic Acid |
| | | | Ligands Molecular Therapy Nucleic Acids, |
| | | | Volume 17, 150-163 |
| Mouse CD3 | R&D SYSTEMS | MAB4841 | rndsystems.com/products/mouse-cd3-antibody- |
| Antibody | | | 17a2_mab4841; |
| (17A2) | | | thermofisher.com/us/en/home/life-science/cell- |
| | | | analysis/cell-analysis-learning-center/immunology- |
| | | | at-work/immunology-protocols/t-cell-activation- |
| | | | anti-cd3-anti-cd28.html |
| CD3 | THERMOFISHER | 16-0032- | thermofisher.com/antibody/product/CD3- |
| Monoclonal | | 82 | Antibody-clone-17A2-Monoclonal/16-0032-82 |
| Antibody | | | |
| (17A2), | | | |
| Functional | | | |
| Grade, | | | |
| eBioscience ™ | | | |
| 145-2C11 | SANTA CRUZ | sc-18871 | scbt.com/p/cd3-e-antibody-145-2c11 |
| monoclonal | BIOTECHNOLOGY | | |
| antibody | | | |
| Foralumab | THERMOFISHER | MA5- | thermofisher.com/antibody/product/Foralumab- |
| (TZLS-0401; | | 41918 | Antibody-Recombinant-Monoclonal/MA5-41918 |
| NI-0401) | | | |

In some embodiments, the anti-CD3 agent comprises an anti-CD3 aptamer. Aptamers can be used in place of antibodies since they function as affinity reagents with high specificity and affinity for their target molecules, similar to antibodies. Aptamers have advantages compared to antibodies such as greater stability, easier and faster chemical synthesis, smaller size, and simpler modification. While antibodies are proteins, aptamers are nucleic acids (DNA or RNA), which can be generated through an in vitro process such as Systematic Evolution of Ligands by Exponential Enrichment (SELEX), allowing for precise control and a consistent supply.

In some embodiments, the anti-CD3 aptamer is dimerized, multimerized, and/or linked to a solid substrate (e.g., tissue culture vessel, bead, etc.), e.g., to achieve agonist activity through CD3 clustering. In some embodiments, the anti-CD3 aptamer is selected from Table 5. In some embodiments, the anti-CD3 aptamer is an RNA aptamer or a DNA aptamer. In some embodiments, the anti-CD3 agent comprises an anti-CD3 antibody selected from Table 4 or an anti-CD3 aptamer selected from Table 5. In some embodiments, the anti-CD3 agent is selected from Table 4, 5, or 8.

TABLE 5
Exemplary anti-CD3 aptamers

| Aptamer name(s) | Reference |
|---|---|
| RNA aptamers (e.g., aptamers | Menon et al. “CD3 aptamers promote expansion |
| 1-13; e.g., aptamer 1 and 12) | and persistence of tumor-reactive T cells for |
| | adoptive T cell therapy in cancer.” Molecular |
| | therapy. Nucleic acids vol. 35.2 102198. 23 Apr. |
| | 2024 |
| DNA aptamers | Zumrut et al. “Ligand-guided selection of |
| | aptamers against T-cell Receptor-cluster of |
| | differentiation 3 (TCR-CD3) expressed on |
| | Jurkat.E6 cells.” Analytical biochemistry vol. 512 |
| | (2016): 1-7. |
| DNA aptamers (e.g., ZUCH-1, ZOKT-2, | Zumrut et al., “Integrating Ligand-Receptor |
| ZUCH-3, ZUCH-4, and ZUCH-5) | Interactions and In Vitro Evolution for |
| | Streamlined Discovery of Artificial Nucleic Acid |
| | Ligands” |
| | Molecular Therapy Nucleic Acids, Volume 17, |
| | 150-163 (2019) |
| Modified DNA aptamer ZUCH1 | Freage et al., “A Homodimeric Aptamer Variant |
| | Generated from Ligand-Guided Selection |
| | Activates the T Cell Receptor Cluster of |
| | Differentiation 3 Complex.” |
| | Molecular Therapy Nucleic Acids, Volume 22, |
| | 167-178 (2020) |

The concentration of the anti-CD3 agent (e.g., anti-CD3 antibody, anti-CD3 aptamer) depends on the specific agent. In some embodiments, the anti-CD3 agent (e.g., anti-CD3 antibody, anti-CD3 aptamer) is present at a concentration of at least 1 ng/mL, at least 10 ng/mL, at least 100 ng/ml, at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, or at least 10 μg/mL, or within a range such as 1-5 μg/mL, 2-10 μg/mL, or 5-20 μg/mL. In some embodiments, the anti-CD3 agent is present at a concentration of about 1 ng/mL, about 10 ng/mL, about 100 ng/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL.

In some embodiments, an anti-CD3 agent is selected based on its ability to specifically bind to CD3. In some embodiments, an anti-CD3 agent is selected based on its ability to specifically bind to CD3ε, CD3γ, CD3δ, and/or CD3ζ (e.g., the extracellular domain of CD3ε, CD3γ, CD3δ, and/or CD3ζ). In some embodiments, an anti-CD3 agent is selected based on its ability to activate CD3 intracellular signalling. In some embodiments, an anti-CD3 agent is selected based on its ability to stimulate a population of DP T progenitor cells differentiates into a population of CD4+CD8− SP T cells, e.g., in combination with IL-7.

### Interleukin-7 (IL-7)

Interleukin-7 (IL-7) is a cytokine, a type of signaling protein, that plays a critical role in the development, survival, and homeostasis of T cells and B cells in the immune system. IL-7 is primarily produced by stromal cells in the bone marrow and thymus and functions by binding to the IL-7 receptor on lymphoid progenitor cells, promoting their proliferation and differentiation. IL-7 is involved with maintaining the survival of naïve and memory T cells. An acceptable concentration range for interleukin-7 (IL-7) in medium used during the differentiation of CD4+CD8+ double-positive T progenitor cells into CD4+CD8− single-positive T cells can be from about 1 ng/ml to about 20 ng/ml, such as at least 1 ng/ml, at least 2 ng/ml, at least 5 ng/ml, at least 10 ng/ml, or 10 to 20 ng/ml. Exemplary concentrations include about 1 ng/ml, about 2 ng/ml, about 3 ng/ml, about 4 ng/mL, about 5 ng/ml, about 6 ng/ml, about 7 ng/ml, about 8 ng/ml, about 9 ng/ml, about 10 ng/ml, about 12 ng/ml, about 15 ng/ml, and about 20 ng/mL. This range encompasses concentrations commonly used in T cell differentiation protocols and allows for optimization based on cell type and experimental conditions.

In some embodiments, the IL-7 is human IL-7. In some embodiments, the IL-7 is mammalian (e.g., human, murine, non-human primate, pig, and/or rat) IL-7. An exemplary amino acid sequence for human interleukin-7 (IL-7) (UniProtKB/Swiss-Prot: P13232) is as follows (SEQ ID NO: 5; aa 1-25 corresponds to the signal peptide, which can be cleaved off during protein processing, resulting in a 152-aa mature IL-7 protein):

MFHVSFRYIFGLPPLILVLLPVASSDCDIEGKDGKQYESVLMVSIDQLL
DSMKEIGSNCLNNEFNFFKRHICDANKEGMFLFRAARKLRQFLKMNSTG
DFDLHLLKVSEGTTILLNCTGQVKGRKPAALGEAQPTKSLEENKSLKEQ
KKLNDLCFLKRLLQEIKTCWNKILMGTKEH

### Fibronectin

Fibronectin is a high-molecular-weight glycoprotein of the extracellular matrix that plays a role in cell adhesion, growth, migration, and differentiation. In the context of T cell development, fibronectin provides an adhesive substrate that supports the survival and maturation of lymphoid progenitor cells. The interaction between fibronectin and integrins on the surface of developing T cells facilitates cellular attachment and signaling, which is involved in efficient differentiation and expansion in vitro. Utilizing fibronectin or recombinant variants thereof as a coating for cell culture substrates enhances the ability of progenitor T cells to adhere, proliferate, and progress through defined stages of T cell lineage commitment.

In some embodiments, the fibronectin is human fibronectin. In some embodiments, the fibronectin is mammalian (e.g., human, murine, non-human primate, pig, and/or rat) fibronectin. In some embodiments, the recombinant variant of fibronectin comprises domains of human fibronectin. In some embodiments, the recombinant variant of fibronectin comprises domains of mammalian (e.g., human, murine, non-human primate, pig, and/or rat) fibronectin. An exemplary amino acid sequence for human fibronectin (UniProtKB/Swiss-Prot: P02751) is as follows (SEQ ID NO: 6; aa 1-31 corresponds to the signal peptide, which can be cleaved off during protein processing, resulting in a 2447-aa mature fibronectin protein):

MLRGPGPGLLLLAVQCLGTAVPSTGASKSKRQAQQMVQPQSPVAVSQSKPGCYDNGKHYQINQQ
WERTYLGNALVCTCYGGSRGFNCESKPEAEETCFDKYTGNTYRVGDTYERPKDSMIWDCTCIGA
GRGRISCTIANRCHEGGQSYKIGDTWRRPHETGGYMLECVCLGNGKGEWTCKPIAEKCFDHAA
GTSYVVGETWEKPYQGWMMVDCTCLGEGSGRITCTSRNRCNDQDTRTSYRIGDTWSKKDNRG
NLLQCICTGNGRGEWKCERHTSVQTTSSGSGPFTDVRAAVYQPQPHPQPPPYGHCVTDSGVVYS
VGMQWLKTQGNKQMLCTCLGNGVSCQETAVTQTYGGNSNGEPCVLPFTYNGRTFYSCTTEGRQ
DGHLWCSTTSNYEQDQKYSFCTDHTVLVQTRGGNSNGALCHFPFLYNNHNYTDCTSEGRRDNM
KWCGTTQNYDADQKFGFCPMAAHEEICTTNEGVMYRIGDQWDKQHDMGHMMRCTCVGNGR
GEWTCIAYSQLRDQCIVDDITYNVNDTFHKRHEEGHMLNCTCFGQGRGRWKCDPVDQCQDSET
GTFYQIGDSWEKYVHGVRYQCYCYGRGIGEWHCQPLQTYPSSSGPVEVFITETPSQPNSHPIQWN
APQPSHISKYILRWRPKNSVGRWKEATIPGHLNSYTIKGLKPGVVYEGQLISIQQYGHQEVTRFDF
TTTSTSTPVTSNTVTGETTPFSPLVATSESVTEITASSFVVSWVSASDTVSGFRVEYELSEEGDEPQY
LDLPSTATSVNIPDLLPGRKYIVNVYQISEDGEQSLILSTSQTTAPDAPPDTTVDQVDDTSIVVRWS
RPQAPITGYRIVYSPSVEGSSTELNLPETANSVTLSDLQPGVQYNITIYAVEENQESTPVVIQQETTG
TPRSDTVPSPRDLQFVEVTDVKVTIMWTPPESAVTGYRVDVIPVNLPGEHGQRLPISRNTFAEVTG
LSPGVTYYFKVFAVSHGRESKPLTAQQTTKLDAPTNLQFVNETDSTVLVRWTPPRAQITGYRLTV
GLTRRGQPRQYNVGPSVSKYPLRNLQPASEYTVSLVAIKGNQESPKATGVFTTLQPGSSIPPYNTE
VTETTIVITWTPAPRIGFKLGVRPSQGGEAPREVTSDSGSIVVSGLTPGVEYVYTIQVLRDGQERD
APIVNKVVTPLSPPTNLHLEANPDTGVLTVSWERSTTPDITGYRITTTPTNGQQGNSLEEVVHADQ
SSCTFDNLSPGLEYNVSVYTVKDDKESVPISDTIIPEVPQLTDLSFVDITDSSIGLRWTPLNSSTIIGY
RITVVAAGEGIPIFEDFVDSSVGYYTVTGLEPGIDYDISVITLINGGESAPTTLTQQTAVPPPTDLRFT
NIGPDTMRVTWAPPPSIDLTNFLVRYSPVKNEEDVAELSISPSDNAVVLTNLLPGTEYVVSVSSVYE
QHESTPLRGRQKTGLDSPTGIDFSDITANSFTVHWIAPRATITGYRIRHHPEHFSGRPREDRVPHSR
NSITLTNLTPGTEYVVSIVALNGREESPLLIGQQSTVSDVPRDLEVVAATPTSLLISWDAPAVTVRY
YRITYGETGGNSPVQEFTVPGSKSTATISGLKPGVDYTITVYAVTGRGDSPASSKPISINYRTEIDKP
SQMQVTDVQDNSISVKWLPSSSPVTGYRVTTTPKNGPGPTKTKTAGPDQTEMTIEGLQPTVEYV
VSVYAQNPSGESQPLVQTAVTNIDRPKGLAFTDVDVDSIKIAWESPQGQVSRYRVTYSSPEDGIHE
LFPAPDGEEDTAELQGLRPGSEYTVSVVALHDDMESQPLIGTQSTAIPAPTDLKFTQVTPTSLSAQ
WTPPNVQLTGYRVRVTPKEKTGPMKEINLAPDSSSVVVSGLMVATKYEVSVYALKDTLTSRPAQG
VVTTLENVSPPRRARVTDATETTITISWRTKTETITGFQVDAVPANGQTPIQRTIKPDVRSYTITGLQ
PGTDYKIYLYTLNDNARSSPVVIDASTAIDAPSNLRFLATTPNSLLVSWQPPRARITGYIIKYEKPGS
PPREVVPRPRPGVTEATITGLEPGTEYTIYVIALKNNQKSEPLIGRKKTDELPQLVTLPHPNLHGPEI
LDVPSTVQKTPFVTHPGYDTGNGIQLPGTSGQQPSVGQQMIFEEHGFRRTTPPTTATPIRHRPRPYP
PNVGEEIQIGHIPREDVDYHLYPHGPGLNPNASTGQEALSQTTISWAPFQDTSEYIISCHPVGTDEE
PLQFRVPGTSTSATLTGLTRGATYNVIVEALKDQQRHKVREEVVTVGNSVNEGLNQPTDDSCFDP
YTVSHYAVGDEWERMSESGFKLLCQCLGFGSGHFRCDSSRWCHDNGVNYKIGEKWDRQGENG
QMMSCTCLGNGKGEFKCDPHEATCYDDGKTYHVGEQWQKEYLGAICSCTCFGGQRGWRCDNC
RRPGGEPSPEGTTGQSYNQYSQRYHQRTNTNVNCPIECFMPLDVQADREDSRE

In some embodiments, the population of DP T progenitor cells are cultured with a substrate coated with fibronectin or a recombinant variant thereof. In some embodiments, the substrate comprises a cell culture vessel. In some embodiments, the fibronectin or a recombinant variant thereof is substantially free of Notch ligand, e.g., Notch ligand is not detectable or present at a concentration of less than 0.1%, less than 0.01%, less than 0.001%, less than 1 ng/mL, less than 1 pg/mL, less than 1 fg/mL, or less than 1 ag/mL.

In some embodiments, the recombinant variant of fibronectin comprises RETRONECTIN. RETRONECTIN (TAKARA) is a 63-kD recombinant human fibronectin fragment (rFN-CH-296), which comprises the cell binding domain (C-domain; type III repeats 8-10), the heparin binding domain (H-domain; type III repeats 12-14), and the CS-1 site of human fibronectin (see e.g., RETRONECTIN product manual at takara.co.kr/file/manual/pdf/T100A_B_e.v1705.pdf). The C-domain of fibronectin binds to VLA-5 (α5β1) integrin receptor and corresponds to type III repeats 8, 9, and/or 10. The H-domain of fibronectin binds to heparin and corresponds to type III repeats 12, 13 and/or 14. The CS-1 site of fibronectin binds to VLA-4 (α4β1) integrin receptor and is found in the IIICS region, between type III repeats 14-15. See e.g., Speziale et al. Fibronectin and Its Role in Human Infective Diseases. Cells 8 (12): 1516 (2019). In some embodiments, the recombinant variant of fibronectin comprises the cell binding domain (C-domain), the heparin binding domain (H-domain), and/or the CS-1 site (binds to VLA-4 integrin receptor) of fibronectin.

The fibronectin or recombinant variant thereof can be coated on the substrate at a concentration of at least 1 μg/mL, at least 5 μg/mL, at least 10 μg/mL, at least 15 μg/mL, at least 20 μg/mL, or at least 50 μg/mL, about 1 μg/mL, about 5 μg/mL, about 10 μg/mL, about 15 μg/mL, about 20 μg/mL, or about 50 μg/mL, or within a range such as 1-5 μg/mL, 5-10 μg/mL, 10-20 μg/mL, or 20-50 μg/mL.

### Anti-CD28 Agents

CD28 is a co-stimulatory receptor expressed on the surface of T cells that plays a role in T cell activation, differentiation, and survival. CD28 is a type I transmembrane glycoprotein that functions as a homodimer, a pair of identical protein subunits linked by a disulfide bond, with each monomer containing an immunoglobulin superfamily (IgSF) domain, a transmembrane domain, and a cytoplasmic tail for signaling. The extracellular IgSF domain contains a binding motif to interact with ligands like CD80 and CD86, while the cytoplasmic tail includes tyrosine-based motifs that initiate intracellular signaling pathways involved in T cell activation. Engagement of CD28 by its natural ligands, or by agonistic agents, provides a secondary; co-stimulatory signal that complements T cell receptor (TCR) signaling through CD3. Specifically; the CD28 signaling pathway is initiated when CD28 on T cells binds CD80 or CD86 on antigen-presenting cells. This binding leads to the phosphorylation of CD28's cytoplasmic tail by kinases like Lck and Fyn. These phosphorylated motifs recruit PI3K and the GADS and Grb2 adaptors, which activate downstream signaling cascades, including the PKC and Ras/MAPK pathways, and activation of NFAT, AP-1, and NF-κB family transcription factors. This activation promotes T-cell proliferation, survival, cytokine production (e.g., IL-2), and T-helper cell differentiation. This co-stimulatory signal is involved with full T cell activation, promoting proliferation, cytokine production, and the prevention of T cell anergy. In T cell differentiation protocols, modulation of CD28 signaling can influence lineage commitment, expansion, and functional maturation of T cell populations. The presence or absence of CD28 stimulation using an anti-CD28 agent during specific stages of in vitro differentiation can be used to direct T cell fate and optimize the generation of desired T cell subsets.

An exemplary amino acid sequence for human CD28 (UniProtKB/Swiss-Prot: P10747) is as follows (SEQ ID NO: 7; extracellular domain bolded (corresponding to amino acids (aa) 19-152; aa 1-18 correspond to signal peptide; aa 153-179 correspond to helical transmembrane domain; aa 180-220 correspond to intracellular domain):

MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

As described herein, the present methods lack an anti-CD28 agent during the method of producing a population of CD4+CD8− single-positive (SP) T cells from a population of DP T progenitor cells. In some embodiments, in the cell culture medium for the CD4+CD8− single-positive (SP) T cells, the anti-CD28 agent is not detectable or present at a concentration of less than 0.1%, less than 0.01%, less than 0.001%, less than 1 ng/mL, less than 1 μg/mL, less than 1 fg/mL, or less than 1 ag/mL. In some embodiments, after differentiation of the population of CD4+CD8− SP T cells, such a population can be cultured in a medium that comprises an anti-CD28 agent, an anti-CD3 agent, and IL-2, e.g., to increase proliferation of the CD4+CD8− SP T cells.

The term “anti-CD28 agent” refers to an agent that is capable of specifically binding to CD28 and activating CD28 intracellular signalling. The anti-CD28 agent can also be referred to as a CD28 agonist. Non-limiting examples of anti-CD28 agents include polypeptides (e.g., antibodies), nucleic acids (e.g., aptamers), and small molecules (see e.g., Esensten et al. “CD28 Costimulation: From Mechanism to Therapy.” Immunity. 2016 May 17; 44 (5): 973-88: Lotze et al. “CD28 co-stimulation: novel insights and applications in cancer immunotherapy.” Nature reviews. Immunology vol. 24.12 (2024): 878-895). In some embodiments, the anti-CD28 agent comprises an antibody, or an antigen-binding domain thereof, or an aptamer. In some embodiments, the anti-CD28 agent specifically binds to human CD28 (see e.g., SEQ ID NO: 7), e.g., the extracellular domain of CD28. In some embodiments, the anti-CD28 agent specifically binds to mammalian (e.g., human, murine, non-human primate, pig, and/or rat) CD28.

In some embodiments, the anti-CD3 agent and the anti-CD28 agent together are capable of specifically binding to CD3 and CD28, respectively, and activating CD3 and CD28 intracellular signaling, respectively. In some embodiments, the anti-CD3 agent and the anti-CD28 agent cross-link CD3 and CD28, to mimic the natural signals received from an antigen-presenting cell and an antigen. Such a dual engagement of CD3 and CD28 can provide a primary signal via the T-cell receptor (TCR)/CD3 complex and a co-stimulatory signal through CD28, leading to T-cell activation, proliferation, and/or cytokine production. In some embodiments, the anti-CD3 agent and the anti-CD28 agent are linked to a substrate, such as magnetic bed or a cell culture vessel, which can increase the crosslinking of CD3 and CD28. In some embodiments, the anti-CD28 agent requires a simultaneous TCR stimulation from an anti-CD3 agent to co-stimulate T cells. In some embodiments, the anti-CD28 agent is a super-agonist, which does not require a simultaneous TCR stimulation from an anti-CD3 agent to co-stimulate T cells.

In some embodiments, the anti-CD28 agent comprises an anti-CD28 antibody or an antigen binding domain thereof. Targeting domains, e.g., anti-CD28 antibodies, antibody reagents, nanobodies, and polypeptides specific for a selected target or substrate are readily available in the art. For example, antibodies for a selected target or substrate (e.g., CD28) can be identified by one of skill in the art using The Antibody Registry (available on the world wide web at antibodyregistry.org); Antibodypedia (available on the world wide web at antibodypedia.com); the Validated Antibody Database (available on the world wide web at labome.com/index.html); or ABCD (available on the world wide web at web.expasy.org/abcd/); or commercial sources such as AbCam, Sigma, ThermoFisher, and BioLegend. One of skill in the art can readily select a targeting domain depending on the target or substrate to be bound. In some embodiments, the anti-CD28 antibody is selected from Table 6. In some embodiments, the anti-CD28 antibody is 37.51, CD28.2, YTH 913.12, Theralizumab, JJ316, or ANC28.1/5D10. In some embodiments, the anti-CD28 antibody is 37.51, CD28.2, or YTH 913.12. In some embodiments, the anti-CD28 antibody is CD28.2. In some embodiments, the anti-CD28 antibody is a super-agonist selected from Theralizumab, JJ316, or ANC28.1/5D10.

TABLE 6
Exemplary anti-CD28 antibodies

| | | catalog/ | |
|---|---|---|---|
| | | accession | |
| mAb name | source | number | links and references |
| CD28 | THERMOFISHER | 14-0281- | thermofisher.com/antibody/product/CD28- |
| Monoclonal | | 82 | Antibody-clone-37-51-Monoclonal/14-0281-82; |
| Antibody | | | thermofisher.com/us/en/home/life-science/cell- |
| (37.51), | | | analysis/cell-analysis-learning- |
| eBioscience ™ | | | center/immunology-at-work/immunology- |
| | | | protocols/t-cell-activation-anti-cd3-anti-cd28.html |
| Human CD28 | CELL | 91920S | cellsignal.com/products/primary- |
| Activating | SIGNALING | | antibodies/human-cd28-activating-cd28-2-mouse- |
| (CD28.2) | TECHNOLOGY | | mab-low-endotoxin-azide-free/91920; |
| Mouse mAb | | | cellsignal.com/products/cellular-assay- |
| (Low | | | kits/human-anti-cd3-cd28-t-cell-activation- |
| Endotoxin, | | | kit/70976 |
| Azide-free) | | | |
| Anti-CD28 | ABSOLUTE | Ab00195- | absoluteantibody.com/product/anti-cd28-yth-913- |
| [YTH | ANTIBODY | 10.0 | 12/Ab00195-10.0_human_igg1/ |
| 913.12], | | | |
| Human IgG1, | | | |
| Kappa | | | |
| Theralizumab | MEDCHEMEXPRESS | HY- | medchemexpress.com/theralizumab.html |
| (TGN1412, | | P9975 | |
| CD28- | | | |
| SuperMAB, | | | |
| and TAB08) | | | |
| (super- | | | |
| agonistic | | | |
| antibody) | | | |
| JJ316 (super- | MEDCHEMEXPRESS | HY- | medchemexpress.com/anti-rat-cd28-antibody- |
| agonistic | | P990819 | jj316.html |
| antibody) | | | |
| Anti-CD28 | LSBIO | LS- | lsbio.com/antibodies/anti-cd28-antibody-clone- |
| Antibody | | C134362 | anc28.1-5d10-elisa-flow-ls- |
| (clone | | | c134362/138043?trid=0 |
| ANC28.1/5D10) | | | |
| (super- | | | |
| agonistic | | | |
| antibody) | | | |

In some embodiments, the anti-CD28 agent comprises an anti-CD28 aptamer. In some embodiments, the anti-CD28 aptamer is dimerized, multimerized, and/or linked to a solid substrate, e.g., to achieve agonist activity through CD28 clustering. In some embodiments, the anti-CD28 aptamer is selected from Table 7. In some embodiments, the anti-CD28 aptamer is an RNA aptamer or a DNA aptamer. In some embodiments, the anti-CD28 agent comprises an anti-CD28 antibody selected from Table 6 or an anti-CD28 aptamer selected from Table 7. In some embodiments, the anti-CD28 agent comprises an anti-CD28 antibody or composition thereof selected from Table 6 or 8 or an anti-CD28 aptamer selected from Table 7.

TABLE 7
Exemplary anti-CD28 aptamers

| | | catalog/accession | |
|---|---|---|---|
| Aptamer name(s) | source | number | links and references |
| Anti-CD28 RNA | CREATIVE | CTApt-531 | creative-biolabs.com/complement- |
| Aptamer-CTApt-531 | BIOLABS | | therapeutics/anti-cd28-aptamer-ctapt- |
| | | | 531-2355.htm |
| Anti-CD28 RNA | CREATIVE | CTApt-532 | creative-biolabs.com/complement- |
| Aptamer-CTApt-532 | BIOLABS | | therapeutics/anti-cd28-aptamer-ctapt- |
| | | | 532-2356.htm |
| Anti-CD28 RNA | CREATIVE | CTApt-M-678 | creative-biolabs.com/complement- |
| Aptamer-CTApt-M- | BIOLABS | | therapeutics/anti-cd28-aptamer-ctapt- |
| 678 | | | m-678-6110.htm |
| Anti-CD28 RNA | CREATIVE | CTApt-M-679 | creative-biolabs.com/complement- |
| Aptamer-CTApt-M- | BIOLABS | | therapeutics/anti-cd28-aptamer-ctapt- |
| 679 | | | m-679-6111.htm |
| RNA Aptamers | Pastor et al., 2013, “CD28 aptamers as powerful immune | | |
| CD28Apt7 (monomer) | response modulators.” Mol Ther Nucleic Acids. 2(6): e98 | | |
| and CD28Apt2 (dimer) | | | |

In some embodiments, the anti-CD3 agent and the anti-CD28 agent are comprised in a single composition, complex, and/or combination, which can increase the crosslinking of CD3 and CD28. In some embodiments, the anti-CD3 agent and the anti-CD28 agent are comprised by a single bispecific agent, such as an anti-CD3/anti-CD28 bispecific antibody or a combination of an anti-CD3 aptamer and an anti-CD28 aptamer. In some embodiments, the anti-CD3 agent and the anti-CD28 agent are in a tetrameric complex or complexes. For example, an anti-CD3 antibody and an anti-CD28 antibody can be in a soluble, four-part antibody complex or “tetramer”: see e.g., U.S. Pat. No. 10,961,506 B2, US Patent Publication 2007/0036783 A1. In some embodiments, the anti-CD3 antibodies are in the same tetramers as the anti-CD28 antibodies. In some embodiments, the anti-CD3 antibodies are in different tetramers than the anti-CD28 antibodies, and the anti-CD3-antibody tetramers and the anti-CD28-antibody tetramers are both provided. In some embodiments, the anti-CD3 agent and the anti-CD28 agent are linked to beads, such as anti-CD3-antibody-and-CD28-antibody-coated beads. In some embodiments, the anti-CD3 antibodies are attached to the same beads as the anti-CD28 antibodies. In some embodiments, the anti-CD3 antibodies are attached to different beads than the anti-CD28 antibodies, and the anti-CD3-antibody beads and the anti-CD28-antibody beads are both provided. In some embodiments, the anti-CD3 agent is selected from Table 4 or 5, and the anti-CD28 agent is selected from Table 6 or 7. Non-limiting examples of compositions, complexes, and combinations comprising the anti-CD3 agent and the anti-CD28 agent are provided in Table 8.

TABLE 8
Exemplary anti-CD3 and anti-CD28 agents

| | | | |
|---|---|---|---|
| | | catalog/ | |
| | | accession | |
| Name | source | number | links and references |

anti-CD3/anti-CD28 bispecific antibodies

| | | | |
|---|---|---|---|
| Recombinant Human Anti- | CREATIVE | T-BSDB- | creative- |
| CD28 × Human Anti-CD3 | BIOLABS | H228 | biolabs.com/bsab/bispecific-cd28- |
| Bispecific Antibody (Tandem | | | cd3-tandem-diabody-12763.htm |
| Diabody) | | | |
| Recombinant Anti-CD28 × | CREATIVE | BSTF-054 | creative- |
| Anti-CD3 Bispecific | BIOLABS | | biolabs.com/bsab/bispecific-cd28- |
| Antibody (scFv-Fc-scFv) | | | cd3-scfv-fc-scfv-7032.htm |
| Anti-CD3/anti-CD28 | HOFFMANN LA | | US Patent Publication |
| bispecific antigen binding | ROCHE INC | | 2023/0416366 A1 (see e.g., SEQ |
| molecules | | | ID NOs: 2-7, 10-15) |
| bsAb CD3-CD28 | INVIVOGEN | bsab-tex-1 | invivogen.com/anti-cd3-cd28-t- |
| | | | cell-expansion |
| 17MA007 CD3-CD28 | MOFITT | | moffitt.org/research- |
| | CANCER | | science/academic-and-industry- |
| | CENTER | | partnerships/office-of- |
| | | | innovation/available- |
| | | | technologies/immunotherapies/17ma007- |
| | | | cd3-cd28-bispecific-antibody- |
| | | | for-the-generation-of-car-t- |
| | | | cells-for-cancer-immunotherapy/ |

anti-CD3/anti-CD28 antibody complexes (tetramers)

| | | | |
|---|---|---|---|
| Rapid-Act T Cell Activation | CELL | 88179 | cellsignal.com/products/cellular- |
| Kit (Human, Anti- | SIGNALING | | assay-kits/rapid-act-t-cell- |
| CD3/CD28) | TECHNOLOGY | | activation-kit-human-anti-cd3- |
| | | | cd28/88179 |
| Human Anti-CD3/CD28 T | CELL | 70976S | cellsignal.com/products/cellular- |
| Cell Activation Kit | SIGNALING | | assay-kits/human-anti-cd3-cd28-t- |
| | TECHNOLOGY | | cell-activation-kit/70976 |
| ImmunoCult ™ Human | STEMCELL | 10971 | stemcell.com/products/immunocult- |
| CD3/CD28 T Cell Activator | | | human-cd3-cd28-t-cell- |
| | | | activator.html |
| Human CD3/CD28 T Cell | CREATIVE | CA- | creative-biolabs.com/car-t/human- |
| Activator-L | BIOLABS | LX146 | cd3-cd28-t-cell-activator-1- |
| | | | 98468.htm |

anti-CD3/anti-CD28 antibody beads or particles

| | | | |
|---|---|---|---|
| Active Max ® Human T cell | ACROBIOSYSTEMS | MBS- | acrobiosystems.com/L-1316- |
| Activation/Expansion | | C001 | CD3%20&%20CD28.html |
| CD3/CD28 Beads, premium | | | |
| grade | | | |
| GMP ActiveMax ® Human T | ACROBIOSYSTEMS | GMP- | acrobiosystems.com/L-1316- |
| cell Activation/Expansion | | MBS001 | CD3%20&%20CD28.html |
| CD3/CD28 Beads | | | |
| Human CD3/CD28 T Cell | BIOLEGEND | 422603 | biolegend.com/nl- |
| Activation Beads | | | nl/products/human-cd3-cd28-t- |
| | | | cell-activation-beads-23351 |
| Cell-Vive ™ GMP | BIOLEGEND | 422605 | biolegend.com/nl-be/products/cell- |
| CD3/CD28 Human T Cell | | | vive-gmp-cd3-cd28-human-t-cell- |
| Activation Beads | | | activation-beads-25529 |
| Enceed ™ T cell Activation | GENSCRIPT | L00899- | genscript.com/cell-activation- |
| Reagent | | 0.5 | products.html |
| Human CD3/CD28 T Cell | MEDCHEMEXPRESS | HY- | medchemexpress.com/inhibitor- |
| Activation Magnetic Beads | | K0353 | kit/human-cd3-cd28-t-cell- |
| | | | activation-magnetic-beads.html |
| T Cell Activation/Expansion | MILTENYI | 130-091- | miltenyibiotec.com/US- |
| Kit, human | BIOTEC | 441 | en/products/t-cell-activation- |
| | | | expansion-kit-human.html |
| Human CD3/CD28 T cell | PROTEINTECH | KMS310 | ptglab.com/products/Human-CD3- |
| Activation Beads Kit | | | CD28-T-cell-Activation-Beads- |
| | | | Kit-KMS310.htm |
| Methods and compositions | QT HOLDINGS | | U.S. Pat. No. 9,790,467 B2 |
| for activation or expansion of | CORP | | |
| T lymphocytes | | | |
| Cloudz ™ T Cell Activation | RNDSYSTEMS | | rndsystems.com/products/cloudz-t- |
| Kit - CD3/CD28 | | | cell-activation-kit-cd3cd28 |
| Dynabeads ™ Human T- | THERMOFISHER | 11161D | thermofisher.com/order/catalog/ |
| Activator CD3/CD28 for T | | | product/11161D |
| Cell Expansion and | | | |
| Activation | | | |
| CTS ™ (Cell Therapy | THERMOFISHER | 40203D | thermofisher.com/order/catalog/ |
| Systems) Dynabeads ™ | | | product/40203D |
| CD3/CD28 | | | |

The concentration of the anti-CD28 agent (e.g., anti-CD28 antibody, anti-CD28 aptamer) depends on the specific agent. In some embodiments, the anti-CD28 agent (e.g., anti-CD28 antibody, anti-CD28 aptamer) is present at a concentration of at least 1 ng/ml, at least 10 ng/ml, at least 100 ng/ml, at least 1 μg/mL, at least 2 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, or at least 10 μg/mL, or within a range such as 1-5 μg/mL. 2-10 μg/mL, or 5-20 μg/mL. In some embodiments, the anti-CD28 agent is present at a concentration of about 1 ng/ml, about 10 ng/ml, about 100 ng/ml, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL.

In some embodiments, the anti-CD3 agent and the anti-CD28 agent are together present at a concentration of at least 1 ng/ml, at least 10 ng/ml, at least 100 ng/ml, at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 20 μg/mL, at least 25 μg/mL, at least 30 μg/mL, at least 40 μg/mL, at least 50 μg/mL, or within a range such as 1-5 μg/mL, 5-10 μg/mL, 10-20 μg/mL, or 20-30 μg/mL. In some embodiments, the anti-CD3 agent and the anti-CD28 agent are together present at a concentration of about 1 ng/mL, about 10 ng/mL, about 100 ng/ml, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 25 μg/mL, about 30 μg/mL, about 40 μg/mL, or about 50 μg/mL.

In some embodiments, an anti-CD28 agent is selected based on its ability to specifically bind to CD28 (e.g., the extracellular domain of CD28). In some embodiments, an anti-CD28 agent is selected based on its ability to activate CD28 intracellular signalling. In some embodiments, an anti-CD28 agent is selected based on its ability to stimulate a population of CD4+CD8− SP T cells to proliferate, e.g., in combination with IL-2 and an anti-CD3 agent.

### Interleukin-2 (IL-2)

Interleukin-2 (IL-2) is a cytokine that plays a role in the growth, proliferation, and differentiation of T cells. IL-2 is produced primarily by activated CD4+ T cells and is involved with the expansion and survival of both effector and regulatory T cell populations. In T cell differentiation protocols, IL-2 can be used to promote the proliferation of single-positive T cells following their specification, supporting the generation of large numbers of functional T cells. The presence of IL-2 in culture, often in combination with anti-CD3 and anti-CD28 agents, enhances T cell activation, expansion, and functional maturation.

In some embodiments, the IL-2 is human IL-2. In some embodiments, the IL-2 is mammalian (e.g., human, murine, non-human primate, pig, and/or rat) IL-2. An exemplary amino acid sequence for human interleukin-2 (IL-2) (UniProtKB/Swiss-Prot: P60568) is as follows (SEQ ID NO: 8; aa 1-20 corresponds to the signal peptide, which can be cleaved off during protein processing, resulting in a 133-aa mature IL-2 protein):

MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT

The IL-2 can be present at a concentration of at least 100 U/mL, at least 150 U/mL, at least 200 U/mL, at least 250 U/mL, at least 300 U/mL, at least 400 U/mL, or at least 500 U/mL, about 100 U/mL, about 150 U/mL, about 200 U/mL, about 250 U/mL, about 300 U/mL, about 400 U/mL, or about 500 U/mL, or within a range such as 100-200 U/mL. 150-250 U/mL. 200-300 U/mL, or 300-500 U/mL. In some embodiments, the IL-2 is present at a concentration of about 200 U/mL.

### CD4+CD8+ Double-Positive T Cells

CD4⁺CD8⁻ double-positive (DP) T cells represent an intermediate stage in T cell development, characterized by the co-expression of both CD4 and CD8 surface markers. These cells arise during thymocyte maturation and are precursors to the generation of single-positive (SP) T cells, which differentiate into either CD4 helper T cells or CD8 cytotoxic T cells. In the context of the disclosed approach, CD4⁺CD8⁻ DP T cells can be derived from hematopoietic stem and progenitor cells (HSPCs) through a directed differentiation protocol.

In one embodiment, the DP T cells are cultured on a substrate coated with fibronectin or a recombinant embodiment, such as RetroNectin®, at a concentration of at least 10 μg/mL, which can support cell adhesion and survival. The culture medium can comprise interleukin-7 (IL-7) at approximately 10 ng/ml and an anti-CD3 antibody, such as clone OKT3 at 5 μg/mL, to promote their differentiation into CD4⁺CD8⁻ SP T cells. This process is carried out in the absence of Notch ligands and anti-CD28 agents, which are conventionally used in T cell maturation protocols, thereby simplifying the differentiation process and can reduce variability. The resulting DP T cells serve as a transitional population, permitting the efficient and scalable production of functional CD4 helper T cells for therapeutic applications.

In some embodiments, a population of CD4⁺CD8⁺; double-positive (DP) T progenitor cells are produced by a method comprising: culturing a population of CD34+ hematopoietic stem and progenitor cells (HSPCs) on a Notch ligand-coated substrate in a medium comprising interleukin-7 (IL-7). In some embodiments, the method of culturing the population of CD34+ HSPCs into the population of CD4 CD8′ double-positive (DP) T progenitor cells further comprises stem cell factor (SCF), Fms-like tyrosine kinase 3 ligand (FLT3L), and/or dexamethasone in the medium.

Such culturing can be performed for a sufficient amount of time, e.g., until the population of CD34+ HSPCs differentiates into the population of DP T proctor progenitor cells. The CD34+ HSPCs can be cultured for at least 7 days, at least 10 days, at least 14 days, at least 21 days, at least 28 days, at least 35 days, or for a period within a range such as 7-10 days, 10-14 days, 14-21 days, 21-28 days, or 28-35 days. For more details about such methods, see e.g., U.S. Pat. No. 11,788,065 B2. Heinze et al., Stem Cell Reports 17, 2610-2628 (2022); the contents of each of which are incorporated herein by reference in their entirety.

In one aspect described herein is a method of producing a population of CD4+CD8− single-positive (SP) T cells, the method comprising: (a) culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells; and (b) culturing the resultant population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells.

In one aspect described herein is a method of producing a population of CD4+CD8− single-positive (SP) T cells, the method comprising: (a) culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells; (b) culturing the resultant population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells; and (c) culturing the resultant population of CD4+CD8− SP T cells in a medium comprising: the anti-CD3 agent, the anti-CD28 agent, and interleukin-2 (IL-2), e.g., to increase proliferation of the population of CD4+CD8− SP T cells.

In some embodiments, the CD4+CD8+ double positive (DP) T progenitor cells are cultured at a concentration of about 1×10\hat{ }5, about 2×10\hat{ }5, about 3×10\hat{ }5, about 4×10\hat{ }5, about 5×10\hat{ }5, about 6×10\hat{ }5, about 7×10\hat{ }5, about 7.5×10\hat{ }5, about 8×10\hat{ }5, about 9×10\hat{ }5, or about 1×10\hat{ }6 cells per mL, or within a range such as 1×10\hat{ }5 to 2×10\hat{ }5 cells per mL, 2×10\hat{ }5 to 5×10\hat{ }5 cells per mL, 5×10\hat{ }5 to 1×10\hat{ }6 cells per mL, or 1×10\hat{ }5 to 1×10\hat{ }6 cells per mL.

In some embodiments, the CD4+CD8+ double positive (DP) T progenitor cells are cultured in Stemspan™ SFEM II medium supplemented with Lymphoid Progenitor Maturation Supplement (STEMCELL TECHNOLOGIES) and Primocin™, which further comprises IL-7 and the anti-CD3 agent, as described herein (see e.g., Table 1). Stemspan™ SFEM II medium comprises bovine serum albumin. insulin (e.g., recombinant human), transferrin (e.g., human transferrin, iron-saturated)), and 2-Mercaptoethanol in Iscove's Modified Dulbecco's Medium (IMDM). IMDM is a basal medium comprising inorganic salts, amino acids, vitamins, glucose, HEPES, phenol red, pyruvic acid, and sodium bicarbonate. Lymphoid Progenitor Maturation Supplement (STEMCELL TECHNOLOGIES) comprises recombinant human cytokines and other additives specifically formulated to support the maturation and differentiation of lymphoid progenitor cells. Primocin™ is a broad-spectrum antimicrobial agent used in cell culture to prevent contamination by bacteria, mycoplasma, and fungi.

In some embodiments, the CD4+CD8+ double positive (DP) T progenitor cells are isolated prior to further steps, e.g., using CD4 and CD8-specific Magnetic-Activated Cell Sorting (MACS) or CD4 and CD8-specific Fluorescence-Activated Cell Sorting (FACS).

### Notch Ligand

Notch ligands are membrane-bound signaling molecules that play a role in cell fate determination during development, including the differentiation of T cells in the thymus. In the context of T cell development, interaction between Notch ligands (such as Delta-like ligand 1 [DLL1] and Delta-like ligand 4 [DLL4]) and Notch receptors on hematopoietic progenitor cells is involved in commitment to the T cell lineage and progression through early developmental stages. Notch signaling influences the balance between T cell and alternative lineage outcomes and is tightly regulated during thymocyte maturation. Modulation or withdrawal of Notch ligand exposure at specific stages can direct the differentiation pathway and impact the generation of distinct T cell subsets, including the transition from double-positive (DP) to single-positive (SP) T cells.

In some embodiments, the Notch ligand is a human Notch ligand. In some embodiments, the Notch ligand is a mammalian (e.g., human, murine, non-human primate, pig, and/or rat) Notch ligand. Non-limiting examples of Notch ligands in mammals include three Delta-like ligands (DLL1, DLL3, DLL4) and two Jagged ligands (JAG1, JAG2). In some embodiments, the Notch ligand is human DLL1, DLL3, DLL4, JAG1, or JAG2. In some embodiments, the Notch ligand is mammalian (e.g., human, murine, non-human primate, pig, and/or rat) DLL1, DLL3, DLL4, JAG1, or JAG2.

In some embodiments, the Notch ligand comprises Delta-like ligand 1 (DLL1) and/or Delta-like ligand 4 (DLL4). In some embodiments, the Notch ligand is human DLL1 or DLL4. In some embodiments, the Notch ligand is mammalian (e.g., human, murine, non-human primate, pig, and/or rat) DLL1 or DLL4. An exemplary amino acid sequence for human Delta-like ligand 1 (DLL1) (UniProtKB/Swiss-Prot: 000548) is as follows (SEQ ID NO: 9; aa 1-17 corresponds to the signal peptide, which can be cleaved off during protein processing, resulting in a 706-aa mature DLL1 protein):

MGSRCALALAVLSALLCQVWSSGVFELKLQEFVNKKGLLGNRNCCRGGA
GPPPCACRTFFRVCLKHYQASVSPEPPCTYGSAVTPVLGVDSFSLPDGG
GADSAFSNPIRFPFGFTWPGTFSLIIEALHTDSPDDLATENPERLISRL
ATQRHLTVGEEWSQDLHSSGRTDLKYSYRFVCDEHYYGEGCSVFCRPRD
DAFGHFTCGERGEKVCNPGWKGPYCTEPICLPGCDEQHGFCDKPGECKC
RVGWQGRYCDECIRYPGCLHGTCQQPWQCNCQEGWGGLFCNQDLNYCTH
HKPCKNGATCTNTGQGSYTCSCRPGYTGATCELGIDECDPSPCKNGGSC
TDLENSYSCTCPPGFYGKICELSAMTCADGPCFNGGRCSDSPDGGYSCR
CPVGYSGFNCEKKIDYCSSSPCSNGAKCVDLGDAYLCRCQAGFSGRHCD
DNVDDCASSPCANGGTCRDGVNDFSCTCPPGYTGRNCSAPVSRCEHAPC
HNGATCHERGHRYVCECARGYGGPNCQFLLPELPPGPAVVDLTEKLEGQ
GGPFPWVAVCAGVILVLMLLLGCAAVVVCVRLRLQKHRPPADPCRGETE
TMNNLANCQREKDISVSIIGATQIKNTNKKADFHGDHSADKNGFKARYP
AVDYNLVQDLKGDDTAVRDAHSKRDTKCQPQGSSGEEKGTPTTLRGGEA
SERKRPDSGCSTSKDTKYQSVYVISEEKDECVIATEV

An exemplary amino acid sequence for human Delta-like ligand 1 (DLL4) (UniProtKB/Swiss-Prot: Q9NR61) is as follows (SEQ ID NO: 10; aa 1-26 corresponds to the signal peptide, which can be cleaved off during protein processing, resulting in a 659-aa mature DLL4 protein):

MAAASRSASGWALLLLVALWQQRAAGSGVFQLQLQEFINERGVLASGRP
CEPGCRTFFRVCLKHFQAVVSPGPCTFGTVSTPVLGTNSFAVRDDSSGG
GRNPLQLPFNFTWPGTFSLIIEAWHAPGDDLRPEALPPDALISKIAIQG
SLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGW
QGRLCNECIPHNGCRHGTCSTPWQCTCDEGWGGLFCDQDLNYCTHHSPC
KNGATCSNSGQRSYTCTCRPGYTGVDCELELSECDSNPCRNGGSCKDQE
DGYHCLCPPGYYGLHCEHSTLSCADSPCFNGGSCRERNQGANYACECPP
NFTGSNCEKKVDRCTSNPCANGGQCLNRGPSRMCRCRPGFTGTYCELHV
SDCARNPCAHGGTCHDLENGLMCTCPAGFSGRRCEVRTSIDACASSPCF
NRATCYTDLSTDTFVCNCPYGFVGSRCEFPVGLPPSFPWVAVSLGVGLA
VLLVLLGMVAVAVRQLRLRRPDDGSREAMNNLSDFQKDNLIPAAQLKNT
NQKKELEVDCGLDKSNCGKQQNHTLDYNLAPGPLGRGTMPGKFPHSDKS
LGEKAPLRLHSEKPECRISAICSPRDSMYQSVCLISEERNECVIATEV

As described herein, the present methods lack a Notch ligand during the method of producing a population of CD4+CD8− single-positive (SP) T cells from a population of DP T progenitor cells. In some embodiments, in the cell culture medium for the CD4+CD8− single-positive (SP) T cells, the Notch ligand is not detectable or present at a concentration of less than 0.1%, less than 0.01%, less than 0.001%, less than 1 ng/mL, less than 1 μg/mL, less than 1 fg/mL, or less than 1 ag/mL.

During culturing of the population of CD34+ HSPCs to differentiate into the population of DP T progenitor cells, the Notch ligand is attached to a solid substrate or support (e.g., a cell culture vessel, beads). In some embodiments, such as small-scale culturing, the Notch ligand is attached to a cell culture vessel. In some embodiments, such as large-scale culturing in a bioreactor, the Notch ligand is attached to beads. To proceed to the next step, the resultant DP T progenitor cells can be removed from the solid substrate or support (e.g., a cell culture vessel, beads) that is coated with Notch ligand and moved to a solid substrate or support (e.g., a cell culture vessel) that is not coated with Notch ligand and/or moved to an environment that does not comprise a solid substrate or support (e.g., beads) coated with Notch ligand, optionally with at least one washing step of the cells, such that Notch ligand is not transferred into the next step of culturing of the population of DP T progenitor cells to differentiate into the population of CD4+CD8− SP T cells, which is performed in the absence of Notch ligand.

### CD34+ Hematopoietic Stem and Progenitor Cells (HSPCs)

CD34+ hematopoietic stem and progenitor cells (HSPCs) are multipotent cells capable of giving rise to all blood cell lineages, including both myeloid and lymphoid cells. HSPCs express the CD34 surface marker and are found in the bone marrow, peripheral blood, and umbilical cord blood. CD34+ HSPCs serve as the starting point for hematopoiesis and are essential for the generation of immune cells, including T cells. B cells, and myeloid cells. In vitro. HSPCs can be derived from pluripotent stem cells and further differentiated into specific immune cell types through the use of defined culture conditions and signaling molecules. The ability to generate and expand HSPCs in culture provides a renewable source of progenitor cells, including for the production of T cells, such as for immunotherapy.

In one embodiment, the population of CD34+ HSPCs are produced by a method comprising: culturing a population of pluripotent stem cells in a medium comprising: bone morphogenetic protein-4 (BMP-4); vascular endothelial growth factor (VEGF); and a glycogen synthase kinase-3β inhibitor, for a sufficient amount of time, e.g., until the population of pluripotent stem cells differentiates into the population of CD34+ HSPCs. In some embodiments, the glycogen synthase kinase-3β inhibitor is CHIR99021.

In some embodiments, the method of culturing the population of pluripotent stem cells to differentiate into the population of CD34+ HSPCs further comprises period(s) of hypoxia and/or period(s) of normoxia. In some embodiments, the method of culturing the pluripotent stem cells further comprises doxycycline in the medium. In some embodiments, the method of culturing the population of pluripotent stem cells to differentiate into the population of CD34+ HSPCs further comprises basic fibroblast growth factor (bFGF) in the medium. In some embodiments, the method of culturing the population of pluripotent stem cells to differentiate into the population of CD34+ HSPCs further comprises stem cell factor (SCF). Fms-like tyrosine kinase 3 ligand (FLT3L). Thrombopoietin (TPO), interleukin 3 (IL-3), interleukin 6 (IL-6), interleukin 11 (IL-11), insulin-like growth factor 1 (IGF-1). Sonic hedgehog protein (SHH), and/or transferrin in the medium.

The pluripotent stem cells can be cultured for at least 7 days, at least 10 days, at least 12 days, at least 14 days, or about 7 days, about 10 days, about 12 days, about 14 days, or for a period within a range such as 7-10 days, 10-12 days, 12-14 days, or 10-14 days. In some embodiments, the CD34+ HSPCs are isolated prior to further steps, e.g., using CD34-specific Magnetic-Activated Cell Sorting (MACS) or CD34-specific Fluorescence-Activated Cell Sorting (FACS). For more details about such methods, see e.g., U.S. Pat. No. 11,788,065 B2, Heinze et al., Stem Cell Reports 17, 2610-2628 (2022): the contents of each of which are incorporated herein by reference in their entirety.

In one aspect described herein is a method of producing a population of CD4+CD8− single-positive (SP) T cells, the method comprising: (a) culturing a population of pluripotent stem cells in a medium comprising: bone morphogenetic protein-4 (BMP-4); vascular endothelial growth factor (VEGF); and a glycogen synthase kinase-3β inhibitor, until the population of pluripotent stem cells differentiates into the population of CD34+ HSPCs; (b) culturing the resultant population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells; and (c) culturing the resultant population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells.

In one aspect described herein is a method of producing a population of CD4+CD8− single-positive (SP) T cells, the method comprising: (a) culturing a population of pluripotent stem cells in a medium comprising: bone morphogenetic protein-4 (BMP-4); vascular endothelial growth factor (VEGF); and a glycogen synthase kinase-3β inhibitor, until the population of pluripotent stem cells differentiates into the population of CD34+ HSPCs; (b) culturing the resultant population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells; (c) culturing the resultant population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells; and (d) culturing the resultant population of CD4+CD8− SP T cells in a medium comprising: the anti-CD3 agent, the anti-CD28 agent, and interleukin-2 (IL-2), e.g., to increase proliferation of the population of CD4+CD8− SP T cells.

### Pluripotent Stem Cells

Pluripotent stem cells are undifferentiated cells capable of giving rise to all three germ layers (endoderm, mesoderm, and ectoderm) thereby permitting the generation of any cell type found in the human body. These cells can be derived from embryonic stem cells (ESCs) or reprogrammed from somatic cells, such as blood cells (e.g., peripheral blood mononuclear cells (PBMCs)), fibroblasts, keratinocytes, or urinary epithelial cells, to create induced pluripotent stem cells (iPSCs). Non-limiting examples of iPSC lines that can be used in the methods described herein include: BUIc2 (XY, EF1a-hSTEMCCA4 loxp lentiviral infection. Cre-excised), BUIc2 TetOn:NICD1, BU2-15-Cr10 TetOn:NICD1 (XY, EF1a-hSTEMCCA4 loxp lentiviral infection. Cre-excised), and/or BU7 (XX, Sendai virus reprogrammed). In some embodiments, the population of pluripotent stem cells express a Notch intracellular domain (NICD), e.g., under an inducible promoter, such as TetON (see e.g., U.S. Pat. No. 11,788,065 B2). iPSCs are particularly advantageous due to their ability to be generated from a patient's own cells (autologous), reducing the risk of immune rejection in therapeutic applications.

In one embodiment, iPSCs are cultured on a substrate coated with extracellular matrix components, such as Matrigel™, in a defined medium like mTeSR1 to maintain their pluripotency. Matrigel™ comprises basement membrane matrix secreted by Engelbreth-Holm-Swarm (EHS) mouse sarcoma cells, which naturally produces a rich extracellular matrix rich in proteins like laminin and collagen IV, as well as growth factors. These cells can then be directed to differentiate into specific lineages (e.g., CD34+ HSPCs) through the addition of signaling molecules, such as bone morphogenetic protein-4 (BMP-4), vascular endothelial growth factor (VEGF), and glycogen synthase kinase-3β inhibitors, which guide their transition into mesodermal or other germ layer-specific progenitors.

In another embodiment, iPSCs are genetically modified to express therapeutic proteins, including but not limited to chimeric antigen receptors (CARs), T cell receptors (TCRs), cytokines (e.g., IFN-γ, TNF-α, IL-2, IL-12, and IL-9, etc.), checkpoint inhibitors (e.g., atezolizumab, avelumab, durvalumab, nivolumab and pembrolizumab, and the like; non-limiting examples of which are provided herein), suicide genes (e.g., herpes simplex virus thymidine kinase (HSV-TK), inducible caspase-9 (iCasp9)), and/or immunomodulatory molecules (e.g., 4-1BBL, CD40L, 4-1BB), prior to differentiation, permitting their use in personalized medicine or off-the-shelf cell therapies. In another embodiment, iPSCs are genetically modified to express at least one polypeptide exogenous to the pluripotent stem cell, including but not limited to chimeric antigen receptors (CARs), checkpoint inhibitors, and/or suicide genes, prior to differentiation. These modifications can be achieved through site-specific integration at loci such as TRAC or AAVS1, ensuring stable expression of the desired genetic elements.

As a non-limiting example, the antigen-binding domain of the CAR can comprise an antibody or an antigen-binding domain thereof (e.g., single-chain variable fragment (scFV)) that specifically binds a tumor antigen (e.g., CD19, BCMA, HER2, EGFR, mesothelin, GD2, PSMA, FAP, etc.), the intracellular signaling domain of the CAR comprises a CD3ζ signaling module, and/or the CAR further comprises a costimulatory signaling domain of CD28 and/or 4-1BB.

### Compositions and Administration

The cells and populations thereof, including CD4+CD8− SP T cells, produced by a method as described herein can be comprised by compositions, such as pharmaceutical compositions, as described further herein. In one aspect, described herein is a pharmaceutical composition comprising a population of cells (e.g., CD4+CD8− SP T cells) produced by a method as described herein and a pharmaceutically acceptable carrier. In one aspect, described herein is a pharmaceutical composition comprising a population of CD4+CD8− SP T cells produced by a method as described herein and a pharmaceutically acceptable carrier. In one aspect, described herein is a pharmaceutical composition comprising a population of Treg cells produced by a method as described herein and a pharmaceutically acceptable carrier. In one aspect, described herein is a pharmaceutical composition comprising a population of CD4+CD8− SP T cells produced by a method as described herein, in combination with CD4-CD8+SP T cells and a pharmaceutically acceptable carrier.

### Formulations

In some embodiments, the technology described herein relates to a pharmaceutical composition comprising a population of cells (e.g., CD4+CD8− SP T cells) produced by a method as described herein as described herein, and optionally a pharmaceutically acceptable carrier. In some embodiments, the active ingredients of the pharmaceutical composition comprise the population of cells (e.g., CD4+CD8− SP T cells) produced by a method as described herein. In some embodiments, the active ingredients of the pharmaceutical composition consist essentially of the population of cells (e.g., CD4+CD8− SP T cells) produced by a method as described herein. In some embodiments, the active ingredients of the pharmaceutical composition consist of the population of cells (e.g., CD4+CD8− SP T cells) produced by a method as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the inactivation and/or killing of the active agent, e.g. population of cells (e.g., CD4+CD8− SP T cells) produced by a method as described herein.

In some embodiments, the pharmaceutical composition comprising the population of cells (e.g., CD4+CD8− SP T cells) produced by a method as described herein can be a parenteral dose form (i.e., administered or occurring elsewhere in the body than the mouth and alimentary canal). Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the population of cells (e.g., CD4+CD8− SP T cells) produced by a method as disclosed within are well known to those skilled in the art. Non-limiting examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosing

In some embodiments, the methods described herein comprise administering an effective amount of compositions described herein, e.g. a population of cells (e.g., CD4+CD8− SP T cells) produced by a method as described herein, to a subject in order to alleviate a symptom of an autoimmune disease, cancer, or infectious disease. As used herein, “alleviating a symptom of an autoimmune disease, cancer, or infectious disease” is ameliorating any condition or symptom associated with the autoimmune disease, cancer, or infectious disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art.

In some embodiments of any of the aspects, the population of cells (e.g., CD4+CD8− SP T cells) produced by a method as described herein is formulated at a dose of at least 1×10\hat{ }3, at least 1×10\hat{ }4, at least 1×10\hat{ }5, at least 1×10⁶ cells/mL, at least 1×10\hat{ }7, at least 1×10\hat{ }8, at least 1×10\hat{ }9, at least 1×10\hat{ }10 cells/mL or about 1×10\hat{ }3, about 1×10\hat{ }4, about 1×10\hat{ }5, about 1×10\hat{ }6 cells/mL, about 1×10\hat{ }7, about 1×10\hat{ }8, about 1×10\hat{ }9, or about 1×10\hat{ }10 cells/mL, or within a range such as about 1×10\hat{ }3 to 1×10\hat{ }6 cells/mL, about 1×10\hat{ }6 to 1×10\hat{ }8 cells/mL, or about 1×10\hat{ }8 to 1×10\hat{ }10 cells/mL.

The term “effective amount” as used herein refers to the amount of the population of cells (e.g., CD4+CD8− SP T cells) needed to alleviate at least one or more symptom of the disease or disorder, and relates to a sufficient amount of pharmacological composition to provide the desired effect. The term “therapeutically effective amount” therefore refers to an amount of the population of cells (e.g., CD4+CD8− SP T cells) that is sufficient to provide a particular anti-autoimmune, anti-cancer, or anti-infection effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the ED50 (the dose therapeutically effective in 50% of the population). The dosage can vary depending upon the dosage form employed and the route of administration utilized. A therapeutically effective dose can be estimated initially from cell culture assays. Also, a dose can be formulated in animal models to achieve a circulating plasma concentration range that includes the IC50 (i.e., the concentration of the population of cells (e.g., CD4+CD8− SP T cells), which achieves a half-maximal inhibition of symptoms) as determined in cell culture, or in an appropriate animal model. Levels in plasma can be measured, for example, by flow cytometry. The effects of any particular dosage can be monitored by a suitable bioassay, e.g., assay for cytokine levels, e.g., in plasma, among others. The dosage can be determined by a physician and adjusted, as necessary, to suit observed effects of the treatment.

The dosage should not be so large as to cause adverse side effects, such as auto-immunity and/or Cytokine Release Syndrome (CRS). Generally, the dosage will vary with the age, condition, and sex of the patient and can be determined by one of skill in the art. The dosage can also be adjusted by the individual physician in the event of any complication.

The efficacy of the population of cells (e.g., CD4+CD8− SP T cells) in, e.g. the treatment of a condition described herein, or to induce a response as described herein can be determined by the skilled clinician. However, a treatment is considered “effective treatment.” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms: or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of autoimmunity, cancer, or infectious disease. When using an experimental animal model, efficacy of treatment is evidenced when a statistically significant change in a marker is observed.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen.

In certain embodiments, an effective dose of a composition comprising the population of cells (e.g., CD4+CD8− SP T cells) as described herein can be administered to a patient once. In certain embodiments, an effective dose of a composition comprising the population of cells (e.g., CD4+CD8− SP T cells) can be administered to a patient repeatedly.

The dosing schedule can vary from once a week to daily depending on a number of clinical factors, such as the subject's sensitivity to the population of cells (e.g., CD4+CD8− SP T cells). The desired dose or amount can be administered at one time or divided into subdoses, e.g., 2-4 subdoses and administered over a period of time, e.g., at appropriate intervals through the day or other appropriate schedule. In some embodiments, administration can be one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising the population of cells (e.g., CD4+CD8− SP T cells) can be administered over a period of time, such as over a 5 minute, 10 minute, 15 minute, 20 minute, or 25 minute period.

In some embodiments, after an initial treatment regimen, the treatments can be administered on a less frequent basis. For example, after treatment biweekly for three months, treatment can be repeated once per month, for six months or a year or longer.

### Administration

A variety of means for administering the compositions described herein to subjects are known to those of skill in the art. Such methods can include, but are not limited to parenteral, intravenous (IV), intramuscular (IM), subcutaneous (SC), transdermal, airway (aerosol), pulmonary, cutaneous, topical, injection, intraosseous (IO), intraperitoneal (IP), intrarectal, intravaginal, intraarticular (IA), or intratumoral administration. Administration can be local or systemic.

In some embodiments of any of the aspects, the population of cells (e.g., CD4+CD8− SP T cells) described herein is administered as a monotherapy, e.g., another treatment for the autoimmune disease, cancer, or infectious disease is not administered to the subject.

The methods described herein can further comprise administering a second agent and/or treatment to the subject, e.g, as part of a combinatorial therapy for autoimmunity. By way of non-limiting example, if a subject is to be treated for autoimmunity, pain, and/or inflammation according to the methods described herein, the subject can also be administered a second agent and/or treatment known to be beneficial for subjects suffering from autoimmunity, pain, and/or inflammation. Examples of such agents and/or treatments include, but are not limited to, non-steroidal anti-inflammatory drugs (NSAIDs-such as aspirin, ibuprofen, or naproxen): corticosteroids, including glucocorticoids (e.g. cortisol, prednisone, prednisolone, methylprednisolone, dexamethasone, betamethasone, triamcinolone, and beclometasone); methotrexate: sulfasalazine: leflunomide: anti-TNF medications; cyclophosphamide: pro-resolving drugs; mycophenolate: or opiates (e.g. endorphins, enkephalins, and dynorphin), steroids, analgesics, barbiturates, oxycodone, morphine, lidocaine, and the like.

In some embodiments of any of the aspects, the methods described herein can further comprise administering a second agent and/or treatment to the subject, e.g, as part of a combinatorial therapy for cancer. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide: alkyl sulfonates such as busulfan, improsulfan and piposulfan: aziridines such as benzodopa, carboquone, meturedopa, and uredopa; ethylenimines and methylmelamines including altretamine, triethylenemelamine, trictylenephosphoramide, triethylenethiophosphoramide and trimethylol melamine: acetogenins (especially bullatacin and bullatacinone): a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation (e.g., pazopanib, sunitinib, sorafenib, regorafenib, cabozantinib, lenvatinib, ponatinib, ziv-aflibercept, axitinib, tivozanib, vandetanib, ramucirumab); and pharmaceutically acceptable salts, acids or derivatives of any of the above.

In some embodiments of any of the aspects, the cancer treatment method further comprises administering an immune checkpoint inhibitor. In some embodiments of any of the aspects, the immune checkpoint inhibitor comprises an immune checkpoint inhibitor antibody. In some embodiments of any of the aspects, the checkpoint inhibitor immunotherapy is an inhibitor of a checkpoint molecule selected from the group consisting of: programmed cell death 1 (PD-1), programmed death-ligand 1 (PD-L1), cytotoxic T-lymphocyte-associated protein 4 (CTLA-4), Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG-3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).

Non-limiting examples of immune checkpoint inhibitors (ICIs) include: pembrolizumab (Keytruda®), nivolumab (Opdivo®), cemiplimab (Libtayo®), spartalizumab, camrelizumab AiRuiKa™), sintilimab (TYVYT®), tislelizumab, toripalimab (Tuoyi™), dostarlimab (JEMPERLI), INCMGA00012, AMP-224, AMP-514 (MEDI0608), atezolizumab (Tecentriq®), avelumab (Bavencio®), envafolimab (KN035), cosibelimab (CK-301), AUNP12, CA-170, BMS-986189, BMS-936559 (MDX-1105), durvalumab (IMFINZI®), tremelimumab, and ipilimumab (Yervoy®). See e.g., U.S. Pat. Nos. 5,811,097, 5,855,887, 6,051,227, 6,682,736, 6,984,720, 7,595,048, 7,605,238, 7,943,743, 8,008,449, 8,217,149, 8,354,509, 8,383,796, 8,728,474, 8,735,553, 8,779,105, 8,779,108, 8,907,053, 8,900,587, 8,952,136, 9,067,999, 9,073,994, 9,683,048, 9,987,500, U.S. Ser. No. 10/160,736, U.S. Ser. No. 10/316,089, U.S. Ser. No. 10/441,655, U.S. Ser. No. 10/590,199, U.S. Ser. No. 11/225,522, US Patent Publication US2014341917; Storz et al., MAbs. 2016 January; 8(1): 10-26; the contents of each of which are incorporated herein by reference in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

In some embodiments of any of the aspects, the methods described herein can further comprise administering a second agent and/or treatment to the subject, e.g. as part of a combinatorial therapy for an infectious disease. Non-limiting examples of a second agent and/or treatment can include an infectious disease therapy selected from the group consisting of: an antimicrobial, an antibiotic, antibacterial, antiviral, antiparasitic, and/or antifungal. The term antimicrobial thus comprises antibacterials, antifungals, and antivirals.

In some embodiments of any of the aspects, the antimicrobial agent can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some embodiments of any of the aspects, the antimicrobial agent can comprise an antibiotic.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

In some embodiments of any of the aspects, the antifungal is selected from the group consisting of: polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles[edit], Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

In some embodiments of any of the aspects, the antiviral is selected from the group consisting of Abacavir, Acyclovir, Adefovir, Amantadine, Ampligen, Amprenavir, antiretroviral, Arbidol, Atazanavir, Atripla, Cidofovir, Combivir, Darunavir, Delavirdine, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscarnet, Fosfonet, Fusion inhibitor, Ibacitabine, Idoxuridine, Imiquimod, Imunovir, Indinavir, Inosine, Integrase inhibitor, Interferon, Interferon type I, Interferon type II, Interferon type III, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Penciclovir, Peramivir, Pleconaril, Podophyllotoxin, Protease inhibitor, Pyramidine, Raltegravir, Reverse transcriptase inhibitor, Ribavirin, Rimantadine, Ritonavir, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine. Additional antivirals known in the art can also be used.

### Methods and Uses

In some embodiments, the methods described herein relate to adoptive cell transfer using the population of cells (e.g., CD4+CD8− single-positive (SP) T cells) produced by a method as described herein. Accordingly, in one aspect described herein is a method of adoptive cell transfer comprising administering an effective amount of the population (e.g., of CD4+CD8− single-positive (SP) T cells) as described herein, or a composition comprising such a population, or a pharmaceutical composition comprising such a population, to a subject in need thereof. In some embodiments, the population of cells (e.g., CD4+CD8− single-positive (SP) T cells) are autologous to the subject. In other embodiments, the population of cells (e.g., CD4+CD8− single-positive (SP) T cells) are allogeneic to the subject.

In some embodiments, the methods described herein relate to treating a subject having or diagnosed as having an autoimmune disease with a population of cells (e.g., CD4+CD8− single-positive (SP) T cells), produced by a method as described herein. Subjects having an autoimmune disease can be identified by a physician using current methods of diagnosing an autoimmune disease. Symptoms and/or complications of an autoimmune disease which characterize these conditions and aid in diagnosis are well known in the art and include but are not limited to, fatigue, joint pain, joint stiffness, muscle weakness, fever, rash, and/or sensitivity to sunlight. Tests that may aid in a diagnosis of an autoimmune disease include, but are not limited to, C-reactive protein (CRP) levels, erythrocyte sedimentation rate (ESR), complete blood count (CBC), comprehensive metabolic panel (CMP), autoantibody tests, biopsies, imaging tests, and/or genetic tests. A family history of an autoimmune disease, or exposure to risk factors for an autoimmune disease (e.g. genetics, infections, environmental factors like smoking and toxic exposures, diet, obesity, gender, age, and/or certain medications) can also aid in determining if a subject is likely to have an autoimmune disease or in making a diagnosis of an autoimmune disease. The compositions described herein can be administered to a subject having or diagnosed as having an autoimmune disease. In some embodiments, the methods described herein comprise administering an effective amount of compositions described herein, e.g. a population of cells (e.g., CD4+CD8− single-positive (SP) T cells) to a subject in order to alleviate a symptom of an autoimmune disease. As used herein, “alleviating a symptom of an autoimmune disease” is ameliorating any condition or symptom associated with the autoimmune disease. As compared with an equivalent untreated control, such reduction is by at least 5%, 10%, 20%, 40%, 50%, 60%, 80%, 90%, 95%, 99% or more as measured by any standard technique.

In some embodiments, the methods described herein relate to treating a subject having or diagnosed as having cancer with a population of cells (e.g., CD4+CD8− single-positive (SP) T cells), produced by a method as described herein. Subjects having cancer can be identified by a physician using current methods of diagnosing cancer. Symptoms and/or complications of cancer which characterize these conditions and aid in diagnosis are well known in the art and include but are not limited to, fatigue, unexplained weight loss or gain, new lumps or swelling, persistent pain, and/or skin changes. Tests that may aid in a diagnosis of cancer include, but are not limited to, biopsies, imaging tests, complete blood count (CBC), tumor marker tests, and/or genetic tests. A family history of cancer, or exposure to risk factors for cancer (e.g. smoking, alcohol, obesity, physical inactivity, sun exposure, exposure to carcinogens, pollution, genetic mutations, age, hormones, and/or chronic inflammation) can also aid in determining if a subject is likely to have cancer or in making a diagnosis of cancer. The compositions described herein can be administered to a subject having or diagnosed as having cancer. In some embodiments, the methods described herein comprise administering an effective amount of compositions described herein, e.g. a population of cells (e.g., CD4+CD8− single-positive (SP) T cells) to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique.

In some embodiments, the methods described herein relate to treating a subject having or diagnosed as having an infectious disease with a population of cells (e.g., CD4+CD8− single-positive (SP) T cells), produced by a method as described herein. Subjects having an infectious disease can be identified by a physician using current methods of diagnosing an infectious disease. Symptoms and/or complications of an infectious disease which characterize these conditions and aid in diagnosis are well known in the art and include but are not limited to, fever, fatigue, cough, sore throat, muscle aches, skin redness, discharge, dehydration, severe respiratory issues, and/or sepsis. Tests that may aid in a diagnosis of an infectious disease include, but are not limited to, pathogen DNA or RNA detection, immunological tests (e.g., for antigens of the pathogen or antibodies against the pathogen), microbiology cultures, complete blood count (CBC), urinalysis, spinal tap, and/or imaging tests. A family history of an infectious disease, or exposure to risk factors for an infectious disease (e.g. direct contact with an infected person or animal, indirect contact via contaminated surfaces or objects, airborne transmission of pathogens, vector-borne transmission by insects or other animals, contaminated water or food, weakened immunity) can also aid in determining if a subject is likely to have an infectious disease or in making a diagnosis of an infectious disease. The compositions described herein can be administered to a subject having or diagnosed as having an infectious disease. In some embodiments, the methods described herein comprise administering an effective amount of compositions described herein, e.g. a population of cells (e.g., CD4+CD8− single-positive (SP) T cells) to a subject in order to alleviate a symptom of an infectious disease. As used herein, “alleviating a symptom of an infectious disease” is ameliorating any condition or symptom associated with the infectious disease. As compared with an equivalent untreated control, such reduction is by at least 5%, 10%, 20%, 40%, 50%, 60%, 80%, 90%, 95%, 99% or more as measured by any standard technique.

In some embodiments, the population of cells (e.g., CD4+CD8− single-positive (SP) T cells) produced by a method as described herein can be used in an in vitro drug screening assay. Non-limiting examples of such in vitro drug screening assays include: screening for immunomodulatory compounds that alter T cell activation or cytokine secretion; evaluating the effects of candidate drugs on T cell proliferation, survival, or exhaustion; assessing the impact of small molecules or biologics on T cell-mediated cytotoxicity; testing checkpoint inhibitor responses; and/or profiling cytokine release in response to various therapeutic agents.

In some embodiments, the population of cells (e.g., CD4+CD8− single-positive (SP) T cells) produced by a method as described herein can be used in an immunological assay. Non-limiting examples of such immunological assays include: mixed lymphocyte reaction (MLR) assays to assess alloreactivity; antigen-specific activation assays; cytokine profiling by ELISA or multiplex bead arrays; flow cytometry-based phenotyping of activation and memory markers; cytotoxicity assays; proliferation assays using CFSE or similar dyes; and T cell receptor (TCR) repertoire analysis.

### Autoimmune Diseases

In various embodiments, a population of cells (e.g., CD4+CD8− single-positive (SP) T cells) produced by a method as described herein can be used to treat autoimmune disease.

“Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Autoantigens, as used herein, are endogenous proteins or fragments thereof that elicit this pathogenic immune response. Autoantigen can be any substance or a portion thereof normally found within a mammal that, in an autoimmune disease, becomes the primary (or a primary) target of attack by the immune system. The term also includes antigenic substances that induce conditions having the characteristics of an autoimmune disease when administered to mammals. Additionally, the term includes peptic subclasses consisting essentially of immunodominant epitopes or immunodominant epitope regions of autoantigens. Immunodominant epitopes or regions in induced autoimmune conditions are fragments of an autoantigen that can be used instead of the entire autoantigen to induce the disease. In humans afflicted with an autoimmune disease, immunodominant epitopes or regions are fragments of antigens specific to the tissue or organ under autoimmune attack and recognized by a substantial percentage (e.g. a majority though not necessarily an absolute majority) of autoimmune attack T-cells.

Autoantigens that are known to be associated with autoimmune disease include myelin proteins with demyelinating diseases, e.g. multiple sclerosis and experimental autoimmune myelitis; collagens and rheumatoid arthritis; insulin, proinsulin, glutamic acid decarboxylase 65 (GAD65); islet cell antigen (ICA512; ICA12) with insulin dependent diabetes.

A common feature in a number of autoimmune related diseases and inflammatory conditions is the involvement of pro-inflammatory CD4+ T cells. These T cells are responsible for the release of inflammatory, Th1 type cytokines. Cytokines characterized as Th1 type include interleukin 2 (IL-2), γ-interferon, TNFα and IL-12. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and in the latter a suppressive response.

Provided herein is a method of treating an autoimmune disease, which comprises administering an effective amount of a composition comprising a population of cells (e.g., CD4+CD8− single-positive (SP) T cells) as described herein to a patient in need thereof. In one embodiment of any one of the methods described, the autoimmune disorder is selected from the group consisting of thyroiditis, type 1 diabetes mellitus, Hashimoto's thyroidits, Graves' disease, celiac disease, multiple sclerosis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, guttate psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritus scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, erythema multiforme, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amyotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic fasciitis, Shulman's syndrome, Felty's syndrome, cyclitis such as chronic cyclitis, heterochromic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangiitis obliterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis, Boeck's disease, enteritis allergica, erythema nodosum leprosum, idiopathic facial paralysis, chronic fatigue syndrome, febris rheumatica, Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired splenic atrophy, vitiligo, toxic-shock syndrome, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy, Wiskott-Aldrich syndrome, ataxia telangiectasia syndrome, angiectasis, autoimmune disorders associated with collagen disease, rheumatism, allergic hypersensitivity disorders, glomerulonephritides, reperfusion injury, ischemic reperfusion disorder, lymphomatous tracheobronchitis, inflammatory dermatoses, dermatoses with acute inflammatory components, and autoimmune uveoretinitis (AUR).

Autoimmune diseases can be mediated by IgG, by inappropriately high levels of IgG, autoreactive IgG, and or immune complex. Non-limiting examples of IgG-mediated autoimmune diseases include Kawasaki disease, Sjogren's disease, Guillain-Barré, inflammatory bowel disease (IBD), Crohn's disease, ulcerative colitis, systemic lupus erythematosus (SLE), lupus arthritis, lupus nephritis, idiopathic thrombocytopenic purpura, rheumatoid arthritis (RA), warm autoimmune hemolytic anemia, heparin induced thrombocytopenia, thrombotic thrombocytopenic purpura, IgA nephritis, pemphigus vulgaris, systemic sclerosis, Wegener's granulomatosis/granulomatosis with polyangiitis, myasthenia gravis, Addison's disease, ankylosing spondylitis, Behçet's syndrome, celiac disease, Goodpasture syndrome/anti-glomerular basement membrane disease, idiopathic membranous glomerulonephritis, Hashimoto's disease, autoimmune pancreatitis, autoimmune hepatitis, primary biliary sclerosis, multiple sclerosis, vasculitis, psoriasis vulgaris, sarcoidosis, type 1 diabetes gestational alloimmune liver disease, Rh disease, ABO incompatibility, neonatal lupus, hemolytic disease of the newborn, neonatal alloimmune thrombocytopenia, neonatal alloimmune neutropenia, and/or neonatal myasthenia gravis.

In some embodiments, “immune complex” and “immunocomplexed antibody” are used interchangeably. In some embodiments, the immune complex is an immune complex of antigen+antigen-specific antibody. In some embodiments and particularly in some assays as described herein, the immune complex is artificial, i.e., does not occur naturally in the mammal. For example, the immune complex may be a multimeric complex of 4-hydroxy-5-iodo-3-nitrophenyl acetic acid (NIP), chicken ovalbumin (OVA), and an anti-NIP antibody. In this context, the anti-NIP antibody is a chimeric IgG antibody that contains a murine variable region specific for 4-hydroxy-5-iodo-3-nitrophenyl acetic acid and an Fc domain from wild-type human IgG1 (see e.g., Claypool, 2004, Mol. Biol. Cell 15:1746-1759).

### Cancers

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

### Infectious Diseases

As used herein, the term “infectious disease” refers to a condition caused by the invasion and multiplication of pathogenic microorganisms in the body, which can include bacteria, viruses, fungi, parasites, and prions. Non-limiting examples of viral pathogens include rhinovirus, COVID-19, influenza virus, Human immunodeficiency virus (HIV), herpes simplex virus (HSV), ebolavirus, hepatitis virus, measles virus, varicella-zoster virus (VZV), or norovirus. Non-limiting examples of bacterial pathogens include Salmonella enterica, Staphylococcus aureus, Escherichia coli (E. coli), Streptococcus pneumoniae, Clostridium difficile (C. diff), Mycobacterium tuberculosis, Neisseria gonorrhoeae, Vibrio cholerae, Listeria monocytogenes, or Yersinia pestis. Non-limiting examples of fungal pathogens include Candida albicans, Candida auris, Aspergillus fumigatus, Trichophyton, Histoplasma capsulatum, Coccidioides immitis/posadasii, or Cryptococcus neoformans. Parasitic pathogens can include protozoa or helminths. Non-limiting examples of parasitic pathogens include Plasmodium species, Giardia duodenalis, Toxoplasma gondii, Trichinella spiralis, Taenia solium, Enterobius vermicularis, or Trypanosoma cruzi. Non-limiting examples of prions include Creutzfeldt-Jakob Disease (CJD) or Bovine Spongiform Encephalopathy (BSE).

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein the term “aptamer” refers to a short segment (e.g., 15 to 100 nucleotides) of nucleic acid (DNA, RNA) that folds into a specific three-dimensional structure to bind to a target molecule with high affinity and specificity.

Autologous refers to cells that are derived from the same individual to whom they are later reintroduced or used. In the context of cell therapy or transplantation, autologous materials eliminate the risk of immune rejection since they originate from the patient's own body.

Allogeneic refers to cells that are derived from a donor who is genetically distinct from the recipient, but of the same species. In the context of transplantation or cell therapy, allogeneic materials can be used to treat the recipient, though they may carry a risk of immune rejection due to genetic differences. Allogeneic cells have the benefit of being able to be prepared and banked in advance of their use.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of an autoimmune disease, cancer, or infectious disease. A subject can be male or female.

A subject can be one who has been previously diagnosed with or identified as suffering from or having a condition in need of treatment (e.g. an autoimmune disease, cancer, or infectious disease) or one or more complications related to such a condition, and optionally, have already undergone treatment for an autoimmune disease, cancer, or infectious disease or the one or more complications related to an autoimmune disease, cancer, or infectious disease. Alternatively, a subject can also be one who has not been previously diagnosed as having an autoimmune disease, cancer, or infectious disease or one or more complications related to an autoimmune disease, cancer, or infectious disease. For example, a subject can be one who exhibits one or more risk factors for an autoimmune disease, cancer, or infectious disease or one or more complications related to an autoimmune disease, cancer, or infectious disease or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein can be a variant of a polypeptide sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a protein or fragment thereof that retains activity of the native or reference polypeptide. A wide variety of, for example, PCR-based, site-specific mutagenesis approaches are known in the art and can be applied by the ordinarily skilled artisan to generate and test artificial variants.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings). In some embodiments, percent sequence identity for an amino acid or nucleic sequence is calculated by dividing the number of identical amino acids or nucleotides, respectively, by the total length of the alignment (including gaps). In some embodiments, the variant amino acid or DNA sequence maintains at least 50%, at least 60%, at least 70%, at least 80%, at least 90%, at least 91%, at least 92%, at least 93%, at least 94%, at least 95%, at least 96%, at least 97%, at least 98%, at least 99%, or more of the function of the native or reference sequence.

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

As used herein, the phrase “maintains the same function”, when used in reference to an enzyme, refers to a polypeptide that catalyzes the same reaction as a reference enzyme with at least 50%, at least 60%, at least 70%, at least 80%, at least 90%, at least 91%, at least 92%, at least 93%, at least 94%, at least 95%, at least 96%, at least 97%, at least 98%, at least 99%, or more of the function of the native or reference enzyme. As used herein, the phrase “maintains the same function”, when used in reference to a nucleic acid encoding a polypeptide, refers to the translated polypeptide maintaining at least 50%, at least 60%, at least 70%, at least 80%, at least 90%, at least 91%, at least 92%, at least 93%, at least 94%, at least 95%, at least 96%, at least 97%, at least 98%, at least 99%, or more of the function of the native or reference polypeptide.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. A wide variety of, site-specific mutagenesis approaches, e.g., Kunkel's method, cassette mutagenesis, PCR site-directed mutagenesis (e.g., traditional PCR, primer extension, or inverse PCR), whole plasmid mutagenesis, in vivo site-directed mutagenesis, CRISPR/Cas-guided mutagenesis, are known in the art and can be applied by the ordinarily skilled artisan to introduce mutations into specific nucleic acid loci. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Braman, Jeff, ed. (2002) In Vitro Mutagenesis Protocols, Methods in Molecular Biology, Vol. 182 (2nd ed.); Khudyakov and Fields (2002), Artificial DNA: Methods and Applications, CRC Press; Hsu et al. (2014), Cell 157 (6): 1262-78; Cerchione et al. (2020) PLOS ONE 15 (4): e0231716; and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the polypeptide genetically modified into the population of pluripotent stem cells described herein is exogenous. In some embodiments of any of the aspects, the polypeptide genetically modified into the population of pluripotent stem cells described herein is ectopic. In some embodiments of any of the aspects, the polypeptide genetically modified into the population of pluripotent stem cells described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an autoimmune disease, cancer, or infectious disease. The term “treating” includes reducing or alleviating at least one adverse effect or symptom of a condition, disease or disorder associated with an autoimmune disease, cancer, or infectious disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a composition or compound as disclosed herein into a subject by a method or route which results in at least partial delivery of the composition, compound, or metabolite thereof at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine. “Contacting” of a cell can be performed in vitro, or ex vivo.

In some embodiments of any of the aspects, the cells can be maintained in culture. As used herein, “maintaining” refers to continuing the viability of a cell or population of cells. A maintained population of cells will have at least a subpopulation of metabolically active cells.

The term “cell culture medium” (also referred to herein as a “culture medium” or “medium”) as referred to herein is a medium for culturing cells containing nutrients that maintain cell viability and support differentiation, maturation, proliferation, and/or activation. The cell culture medium may contain any of the following in an appropriate combination: salt(s), buffer(s), amino acids, glucose or other sugar(s), antibiotics, serum or serum replacement, and other components such as peptide growth factors, etc. The appropriate cell culture media, for a particular cell type, is known to those skilled in the art. As a non-limiting example, the cells may be cultured in culture medium comprising conditioned medium, non-conditioned medium, or embryonic stem cell medium. Examples of suitable conditioned medium include IMDM, DMEM, or αMEM, conditioned with embryonic fibroblast cells (e.g. human embryonic fibroblast cells or mouse embryonic fibroblast cells), or equivalent medium. Examples of suitable non-conditioned medium include ISCOVE'S MODIFIED DULBECCO'S MEDIUM (IMDM), DMEM, or αMEM, or equivalent medium. The culture medium may comprise serum (e.g. bovine serum, fetal bovine serum, calf bovine serum, horse serum, human serum, or an artificial serum substitute) or it may be serum free. Further non-limiting examples of cell culture media are described herein, including in the Examples.

As used herein in relation to cell culture, the term “substrate” or “solid support” refers to a material or composition adapted for use in the propagation and cultivation of cells. Non-limiting examples of substrates or solid supports used in the cell culture methods described herein include cell culture vessels (e.g., polystyrene, polypropylene, and/or glass-bottom or the like plates, wells, dishes, etc.), beads (e.g., magnetic, agarose, polymer, and/or glass beads), slides, cellulose membranes, hydrogels, and the like.

In some embodiments of any of the aspects, methods described herein comprise isolating, collecting, or concentrating a cell or population thereof. As used herein the terms “isolate,” “collect,” “concentrate”, “purify” and “extract” are used interchangeably and refer to a process whereby a cell (e.g., a CD4+CD8− single-positive (SP) T cell) is removed from a source, such as a fluid (e.g., culture medium). In some embodiments of any of the aspects, methods of isolation, collection, concentration, purification, and/or extraction comprise a reduction in the amount of at least one heterogeneous element (e.g., other non-target cells). In some embodiments of any of the aspects, methods of isolation, collection, concentration, purification, and/or extraction reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogeneous elements. The presence of cells of interest can be assayed by any appropriate method including flow cytometry.

As used herein, the term “specific binding” refers to a chemical or physical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance.

The term “statistically significant” or “significantly” refers to statistical significance and generally means a two standard deviation (2SD) or greater difference or a p-value of less than 0.05.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The section headings used herein are for organizational purposes only and are not to be construed as limiting the subject matter described.

Some embodiments of the technology described herein can be defined according to any of the following numbered paragraphs:
1. A method of producing a population of CD4+CD8− single-positive (SP) T cells, the method comprising: culturing a population of CD4+CD8+ double positive (DP) T progenitor cells in the absence of: Notch ligand and an anti-CD28 agent, and in a medium comprising interleukin-7 (IL-7) and an anti-CD3 agent until the population of DP T progenitor cells differentiates into the population of CD4+CD8− SP T cells.
2. The method of paragraph 1, wherein the anti-CD3 agent specifically binds and activates CD3 intracellular signalling.
3. The method of paragraph 1 or 2, wherein the anti-CD3 agent is not bound to a substrate.
4. The method of paragraph 1-3, wherein the anti-CD3 agent comprises an anti-CD3 antibody or an antigen binding domain thereof.
5. The method of paragraph 4, wherein the anti-CD3 antibody is selected from Table 4 (e.g., OKT3, Otelixizumab, Teplizumab, HIT3a, UCHT1, 17A2, 145-2C11, or Foralumab).
6. The method of any one of paragraphs 1-3, wherein the anti-CD3 agent comprises an anti-CD3 aptamer.
7. The method of paragraph 6, wherein the anti-CD3 aptamer is selected from Table 5.
8. The method of any one of paragraphs 1-7, wherein the anti-CD3 agent is present at a concentration of about 5 μg/mL.
9. The method of any one of paragraphs 1-8, wherein the IL-7 is present at a concentration of about 10 ng/mL.
10. The method of any one of paragraphs 1-9, wherein the population of DP T progenitor cells are cultured for at least 14 days.
11. The method of any one of paragraphs 1-10, wherein the population of DP T progenitor cells are cultured with a substrate coated with fibronectin or a recombinant variant thereof.
12. The method of paragraph 11, wherein the fibronectin or recombinant variant thereof is coated on the substrate at a concentration of at least 10 μg/mL.
13. The method of any one of paragraphs 1-12, further comprising culturing the population of CD4+CD8− SP T cells in a medium comprising: the anti-CD3 agent, the anti-CD28 agent, and interleukin-2 (IL-2).
14. The method of any one of paragraphs 1-13, wherein the anti-CD28 agent specifically binds and activates CD28 intracellular signalling.
15. The method of any one of paragraphs 1-14, wherein the anti-CD28 agent comprises an anti-CD28 antibody or an antigen binding domain thereof.
16. The method of paragraph 15, wherein the anti-CD28 antibody is selected from Table 6.
17. The method of any one of paragraphs 1-14, wherein the anti-CD28 agent comprises an anti-CD28 aptamer.
18. The method of paragraph 17, wherein the anti-CD28 aptamer is selected from Table 7.
19. The method of any one of paragraphs 1-18, wherein the anti-CD3 agent and the anti-CD28 agent are present at a concentration of about 25 μg/mL.
20. The method of any one of paragraphs 1-19, wherein the anti-CD3 agent and the anti-CD28 agent are comprised by a single bispecific agent.
21. The method of any one of paragraphs 1-20, wherein the anti-CD3 agent and the anti-CD28 agent are linked to a substrate.
22. The method of any one of paragraphs 1-21, wherein the substrate is a magnetic bead or a cell culture vessel.
23. The method of any one of paragraphs 1-22, wherein the anti-CD3 agent and the anti-CD28 agent are in a tetrameric complex.
24. The method of any one of paragraphs 1-23, wherein the anti-CD3 agent and the anti-CD28 agent are selected from Table 8.
25. The method of any one of paragraphs 1-24, wherein the IL-2 is present at a concentration of about 200 U/mL.
26. The method of any one of paragraphs 1-25, wherein the population of CD4+CD8− SP T cells is cultured for at least 14 days to at most 140 days.
27. The method of any one of paragraphs 1-26, wherein the culturing increases proliferation of the population of CD4+CD8− SP T cells.
28. The method of any one of paragraphs 1-27, wherein the population of CD4+CD8− SP T cells undergo at least 2, at least 3, at least 4, at least 5, or at least 6 population doublings.
29. The method of any one of paragraphs 1-28, wherein the population of CD4+CD8− SP T cells comprises at least 40%, at least 50%, at least 60% or more CD4+CD8− SP T cells; and/or wherein the population of CD4+CD8− SP T cells comprises at least 1×10⁷, at least 1×10⁸, at least 1×10⁹ or more total CD4+CD8− SP T cells.
30. The method of any one of paragraphs 1-29, further comprising isolating the population of CD4+CD8− SP T cells.
31. The method of any one of paragraphs 1-30, wherein the CD4+CD8− SP T cells are CD3+ and TCRαβ+.
32. The method of any one of paragraphs 1-31, wherein the CD4+CD8− SP T cells are CD62L+, CD5+, CCR7+, MHC I+, CD69+, and/or CD25+.
33. The method of any one of paragraphs 1-32, wherein the CD4+CD8− SP T cells express T-helper-inducing POZ-Kruppel-like factor (ThPOK) and/or T cell receptor alpha chain constant (TRAC).
34. The method of any one of paragraphs 1-33, wherein the CD4+CD8− SP T cells are capable upon stimulation of secreting at least one of the following: interferon-γ (IFNγ), tumor necrosis factor-α (TNFα), interleukin-2 (IL-2), interleukin 4 (IL-4), interleukin 5 (IL-5), interleukin 6 (IL-6), interleukin 8 (IL-8), interleukin 9 (IL-9), interleukin 10 (IL-10), interleukin 13 (IL-13), interleukin 17a (IL-17a), interleukin 17f (IL-17f), interleukin 22 (IL-22), amphiregulin (AREG), granzyme B, macrophage inflammatory protein-1 alpha (MIP1a), and/or macrophage inflammatory protein-1 beta (MIP1β), or any combination thereof.
35. The method of any one of paragraphs 1-34, wherein at least 90% of the CD4+CD8− SP T cells in the population are capable upon stimulation of secreting IFNγ and TNFα.
36. The method of any one of paragraphs 1-35, further comprising, prior to culturing the population of DP T progenitor cells, a step of: culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells.
37. The method of paragraph 36, wherein the Notch ligand comprises Delta-like ligand 1 (DLL1) and/or Delta-like ligand 4 (DLL4).
38. The method of paragraph 36 or 37, wherein the CD34+ HSPCs are cultured for at least 28 days.
39. The method of any one of paragraphs 36-38, further comprising, prior to culturing the population of CD34+ HSPCs, a step of: culturing a population of pluripotent stem cells in a medium comprising: bone morphogenetic protein-4 (BMP-4); vascular endothelial growth factor (VEGF); and a glycogen synthase kinase-3p inhibitor, until the population of pluripotent stem cells differentiates into the population of CD34+ HSPCs.
40. The method of paragraph 39, wherein the population of pluripotent stem cells comprises induced pluripotent stem cells (iPSCs).
41. The method of paragraph 39 or 40, wherein the population of pluripotent stem cells is cultured for at least 12 days.
42. The method of any one of paragraphs 39-41, further comprising isolating the CD34+ HSPCs.
43. The method of any one of paragraphs 39-42, further comprising, prior to culturing the population of pluripotent stem cells, genetically modifying the population of pluripotent stem cells to express a polypeptide exogenous to the pluripotent stem cell.
44. The method of paragraph 43, wherein the polypeptide comprises a chimeric antigen receptor (CAR).
45. The method of paragraph 43 or 44, wherein a nucleic acid encoding the polypeptide is integrated at the T-cell receptor alpha constant locus (TRAC) locus or the adeno-associated virus integration site 1 (AAVS1) locus of the genome of the pluripotent stem cell.
46. The method of paragraph 44 or 45, wherein the antigen-binding domain of the CAR comprises a single-chain variable fragment that specifically binds CD19.
47. The method of any one of paragraphs 44-46, wherein the intracellular signaling domain of the CAR comprises a CD3ζ signaling module.
48. The method of any one of paragraphs 44-47, wherein the CAR further comprises a costimulatory signaling domain of CD28 and/or 4-1BB.
49. The method of any one of paragraphs 1-48, further comprising differentiating the population of CD4+CD8− SP T cells into a population of regulatory T cells by culturing in a medium comprising transforming growth factor-beta (TGF-0), interleukin-2 (IL-2), retinoic acid, and rapamycin.
50. A population of CD4+CD8− SP T cells produced using the method of any one of paragraphs 1-49.
51. A population of regulatory T cells produced using the method of paragraph 49.
52. A composition comprising the population of paragraph 50 or 51.
53. A pharmaceutical composition comprising the population of paragraph 50 or 51 and a pharmaceutically acceptable carrier.
54. A method of adoptive cell transfer comprising administering an effective amount of the population of paragraph 50 or 51, or the composition of paragraph 52, or the pharmaceutical composition of paragraph 53, to a subject in need thereof.
55. The method of paragraph 54, wherein the population of cells is autologous to the subject.
56. The method of paragraph 54, wherein the population of cells is allogeneic to the subject.
57. A method of treating an autoimmune disease comprising administering an effective amount of the population of paragraph 50 or 51, or the composition of paragraph 52, or the pharmaceutical composition of paragraph 53, to a subject in need thereof.
58. A method of treating cancer comprising administering an effective amount of the population of paragraph 50 or 51, or the composition of paragraph 52, or the pharmaceutical composition of paragraph 53, to a subject in need thereof.
59. A method of treating an infectious disease comprising administering an effective amount of the population of paragraph 50 or 51, or the composition of paragraph 52, or the pharmaceutical composition of paragraph 53, to a subject in need thereof.
60. Use of the population of paragraph 50 or 51 in an in vitro drug screening assay.
61. Use of the population of paragraph 50 or 51 in an immunological assay.

Some embodiments of the technology described herein can be defined according to any of the following numbered paragraphs:
1. A method of producing a population of CD4+CD8− single-positive (SP) T cells, the method comprising:
   culturing a population of CD4+CD8+ double positive (DP) T progenitor cells in the absence of Notch ligand and an anti-CD28 agent, and in a medium comprising interleukin-7 (IL-7) and an anti-CD3 agent until the population of DP T progenitor cells differentiates into the population of CD4+CD8− SP T cells.
2. The method of paragraph 1, wherein the anti-CD3 agent specifically binds and activates CD3 intracellular signalling.
3. The method of paragraph 1, wherein the anti-CD3 agent is not bound to a substrate.
4. The method of paragraph 1, wherein the anti-CD3 agent comprises an anti-CD3 antibody or an antigen binding domain thereof, or an anti-CD3 aptamer.
5. The method of paragraph 4, wherein the anti-CD3 antibody is selected from OKT3, Otelixizumab, Teplizumab, HIT3a, UCHT1, 17A2, 145-2C11, or Foralumab.
6. The method of paragraph 4, wherein the anti-CD3 antibody is OKT3.
7. The method of paragraph 1, wherein the anti-CD3 agent is present at a concentration of about 5 μg/mL.
8. The method of paragraph 1, wherein the IL-7 is present at a concentration of about 10 ng/mL.
9. The method of paragraph 1, wherein the population of DP T progenitor cells are cultured for at least 14 days.
10. The method of paragraph 1, wherein the population of DP T progenitor cells are cultured with a substrate coated with fibronectin or a recombinant variant thereof.
11. The method of paragraph 10, wherein the fibronectin or recombinant variant thereof is coated on the substrate at a concentration of at least 10 μg/mL.
12. The method of paragraph 1, further comprising culturing the population of CD4+CD8− SP T cells in a medium comprising: the anti-CD3 agent, the anti-CD28 agent, and interleukin-2 (IL-2).
13. The method of paragraph 12, wherein the anti-CD28 agent specifically binds and activates CD28 intracellular signalling.
14. The method of paragraph 12, wherein the anti-CD28 agent comprises an anti-CD28 antibody or an antigen binding domain thereof, or an anti-CD28 aptamer.
15. The method of paragraph 12, wherein the anti-CD3 agent and the anti-CD28 agent are comprised by a single bispecific agent, are linked to a substrate, and/or are in a tetrameric complex.
16. The method of paragraph 12, wherein the anti-CD3 agent and the anti-CD28 agent comprise anti-CD3 antibodies and anti-CD28 antibodies in tetrameric complexes.
17. The method of paragraph 12, wherein the anti-CD3 agent and the anti-CD28 agent are present at a concentration of about 25 μg/mL.
18. The method of paragraph 12, wherein the IL-2 is present at a concentration of about 200 U/mL.
19. The method of paragraph 12, wherein the culturing increases proliferation of the population of CD4+CD8− SP T cells.
20. The method of paragraph 1, wherein the population of CD4+CD8− SP T cells is cultured for at least 14 days to at most 140 days.
21. The method of paragraph 1, wherein the population of CD4+CD8− SP T cells comprises at least 40%, at least 50%, at least 60%, or more CD4+CD8− SP T cells; and/or wherein the population of CD4+CD8− SP T cells comprises at least 1×10⁷, at least 1×10⁸, at least 1×10⁹ or more total CD4+CD8− SP T cells.
22. The method of paragraph 1, wherein the CD4+CD8− SP T cells are CD3+ and TCRαβ+; wherein the CD4+CD8− SP T cells are CD62L+, CD5+, CCR7+, MHC I+, CD69+, and/or CD25+; wherein the CD4+CD8− SP T cells express T-helper-inducing POZ-Kruppel-like factor (ThPOK) and/or T cell receptor alpha chain constant (TRAC); wherein the CD4+CD8− SP T cells are capable upon stimulation of secreting at least one of the following: interferon-γ (IFNγ), tumor necrosis factor-α (TNFα), interleukin-2 (IL-2), interleukin 4 (IL-4), interleukin 5 (IL-5), interleukin 6 (IL-6), interleukin 8 (IL-8), interleukin 9 (IL-9), interleukin 10 (IL-10), interleukin 13 (IL-13), interleukin 17a (IL-17a), interleukin 17f (IL-17f), interleukin 22 (IL-22), amphiregulin (AREG), granzyme B, macrophage inflammatory protein-1 alpha (MIP1a), and/or macrophage inflammatory protein-1 beta (MIP10), or any combination thereof; and/or wherein at least 90% of the CD4+CD8− SP T cells in the population are capable upon stimulation of secreting IFNγ and TNFα.
23. The method of paragraph 1, further comprising, prior to culturing the population of DP T progenitor cells, a step of: culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells.
24. The method of paragraph 23, further comprising, prior to culturing the population of CD34+ HSPCs, a step of: culturing a population of pluripotent stem cells in a medium comprising: bone morphogenetic protein-4 (BMP-4); vascular endothelial growth factor (VEGF); and a glycogen synthase kinase-3p inhibitor, until the population of pluripotent stem cells differentiates into the population of CD34+ HSPCs.
25. The method of paragraph 24, further comprising, prior to culturing the population of pluripotent stem cells, genetically modifying the population of pluripotent stem cells to express a polypeptide exogenous to the pluripotent stem cell.
26. The method of paragraph 25, wherein the polypeptide comprises a chimeric antigen receptor (CAR).
27. A population of CD4+CD8− SP T cells produced using the method of paragraph 25.
28. A composition comprising the population of paragraph 27.
29. A pharmaceutical composition comprising the population of paragraph 27 and a pharmaceutically acceptable carrier.
30. A method of treating an autoimmune disease, cancer, or infectious disease, the method comprising administering an effective amount of the population of paragraph 27 to a subject in need thereof.

### EXAMPLES

The present technology will be further described in the following examples, which do not limit the scope of the present disclosure.

### Example 1: Robust Generation of CD4 T Helper Cells from Human iPSC

In the human thymus, ap T cell progenitors differentiate into both CD4+ helper cells and CD8+ cytotoxic cells. T cell progenitors derived from induced pluripotent stem cells fail to effectively differentiate into CD4+ helper cells. The present disclosure describes a key role for the withdrawal of Notch ligand during the final step of stimulation to prompt T cell maturation allowing access to the CD4 lineage in iPSC T cells (iT cells). These CD4+ iT cells produce signature CD4 cytokines and transcription factors and show similarity to human blood CD4 T cells.

T cells are a key component of the human immune system, acting as cytotoxic effectors and helpers which modulate other immune/inflammatory functions. T cells originate as hematopoietic progenitor cells which migrate from the bone marrow to the thymus. Transition from hematopoietic progenitor to a CD4/CD8 double negative lymphoid progenitor to a CD4/CD8 double positive progenitor takes place in the Notch ligand-rich environment of the thymic cortex, where the cells are maintained by survival cytokine IL7. Double positive progenitor cells are then signaled to mature to single positive thymocytes by T cell receptor (TCR) binding to major histocompatibility complexes presented by thymic epithelial cells in a process termed “positive selection”. While the initiation of positive selection occurs in the thymic cortex, positively selected DP thymocytes migrate through the cortico-medullary junction into the medulla. After migration to the medulla the single positive (SP) semimature T cells undergo negative selection in which highly self-reactive clones undergo apoptosis, or in the case of some strongly signaled CD4 clones are diverted to an anti-inflammatory regulatory T cell fate.

Despite work implicating strength of TCR signaling, duration of TCR signaling, and co-receptor or cytokine signals the elements resulting in the divergence of these 2 fundamental T cell lineages, CD8 vs CD4, remain in question. IL7 is required for CD8 SP cell survival, and both CD4 and CD8 SP cells require the binding of the TCR to MHC (MHC II and MHC I respectively) loaded with a compatible antigen.

With the advent of human induced pluripotent stem cells, which have the capability to differentiate to any human cell with the correct sequence of signals, it has become possible to follow T cell development and generate lymphoid progenitors. While robust generation of the CD4+/CD8+ double positive (DP) progenitor cells have been demonstrated, most protocols have resulted primarily in the production of CD8 cytotoxic lymphocytes. CD4 T cells are the second arm of T cell immunity, with CD4 T cell cytokines being a requirement for the most effective T cell therapies. The present disclosure provides a highly scalable, feeder free platform for the generation of iPSC derived CD4+ T cells.

T cells are the basis of several advanced cell therapies, including Chimeric Antigen Receptor (CAR) T cell approaches that target cancers and T regulatory cell and CAR T regulatory anti-inflammatory treatments for autoimmune and aberrant inflammatory conditions. Conventional CAR T immunotherapy approaches use either autologous T cells derived from the patient being treated or allogeneic T cells taken from healthy donors. A key drawback of autologous CAR T therapy is time and cost to treatment, while donor-to-donor variation in starting T cell material can result in differences in outcome for patients. The ratio of CD4 to CD8 T cells has been implicated in effective response, along with specific populations. Allogeneic CAR T can be prepared and banked in advance, but carry a risk of immune rejection; however universal donor edits are being developed to reduce immunogenicity. These same edits are applicable to iPSC derived T cells as well; however, current protocols primarily allow for CD8 iT cell generations at scale, while a mix of CD4 and CD8 T cells are required for optimal therapy.

### Notch Withdrawal Allows for CD4 Lineage Emergence.

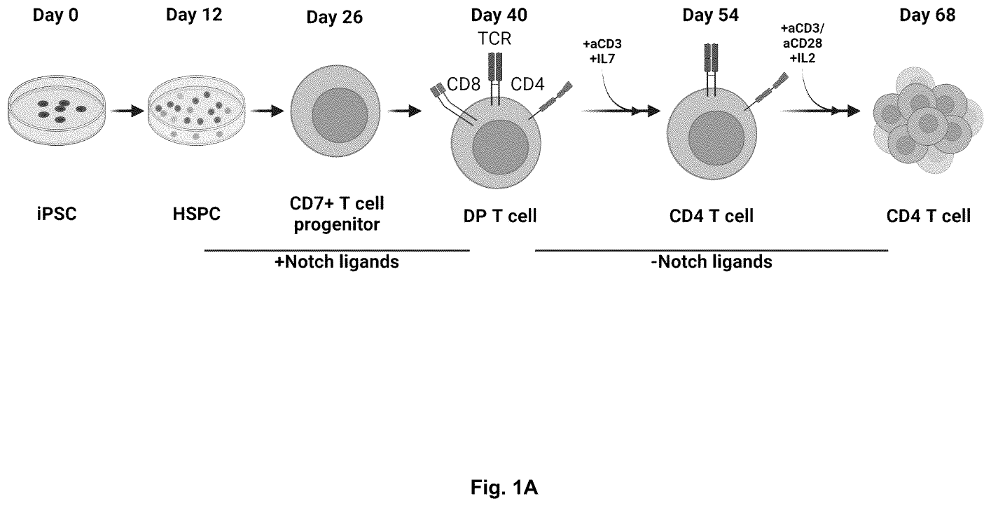

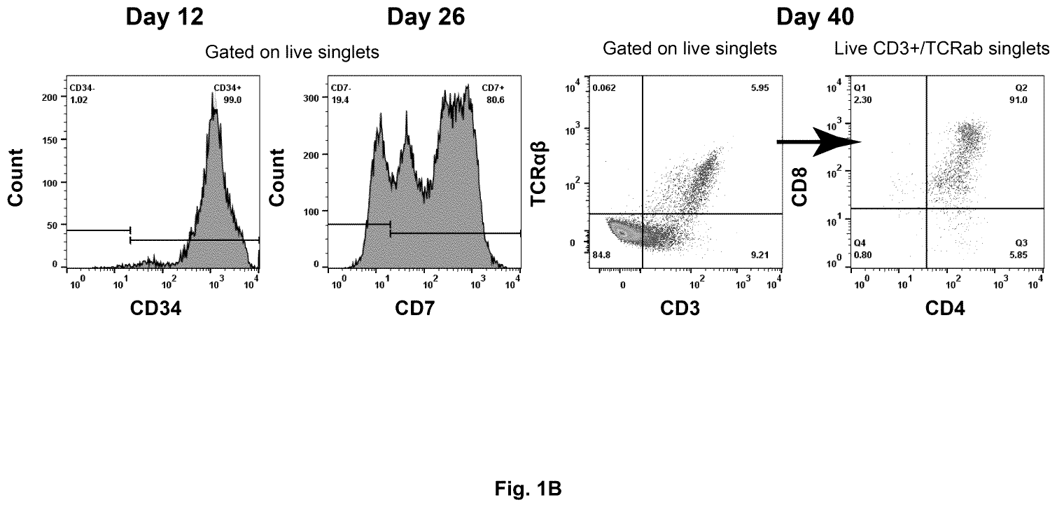

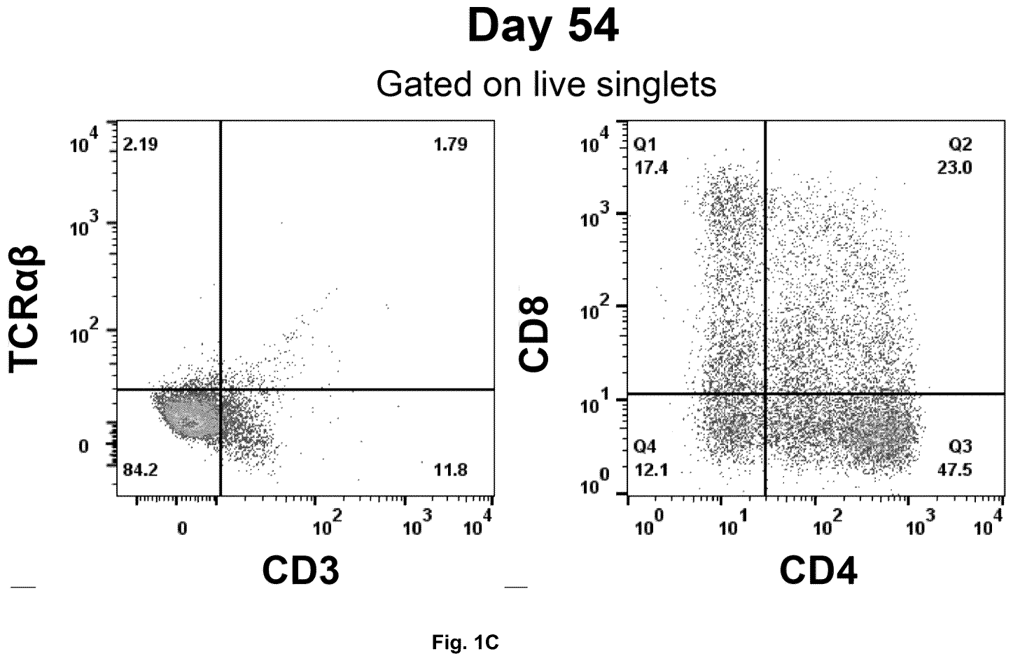

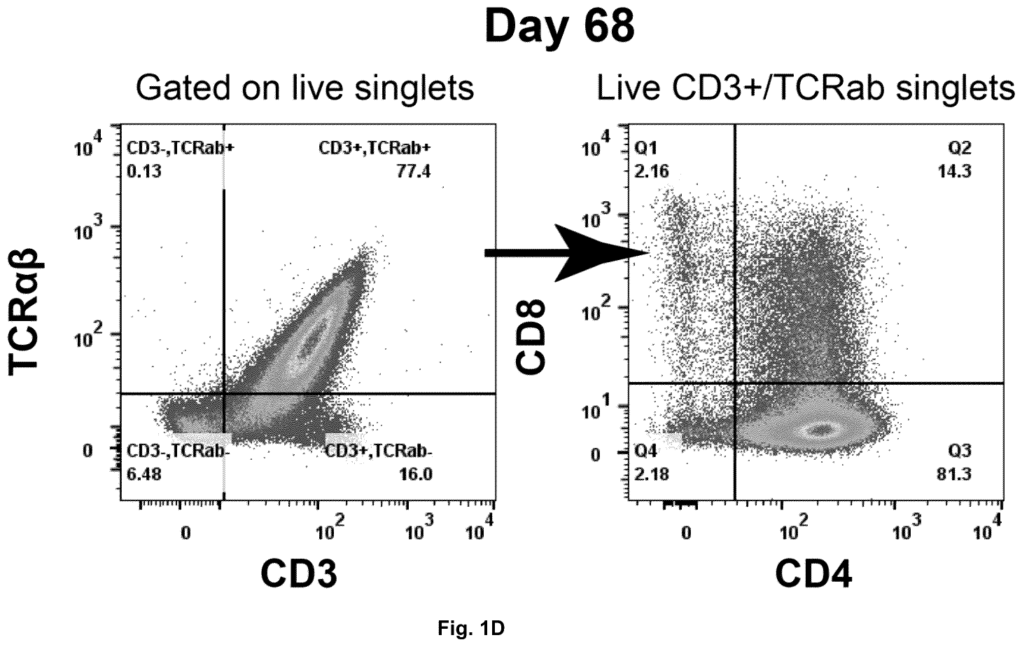

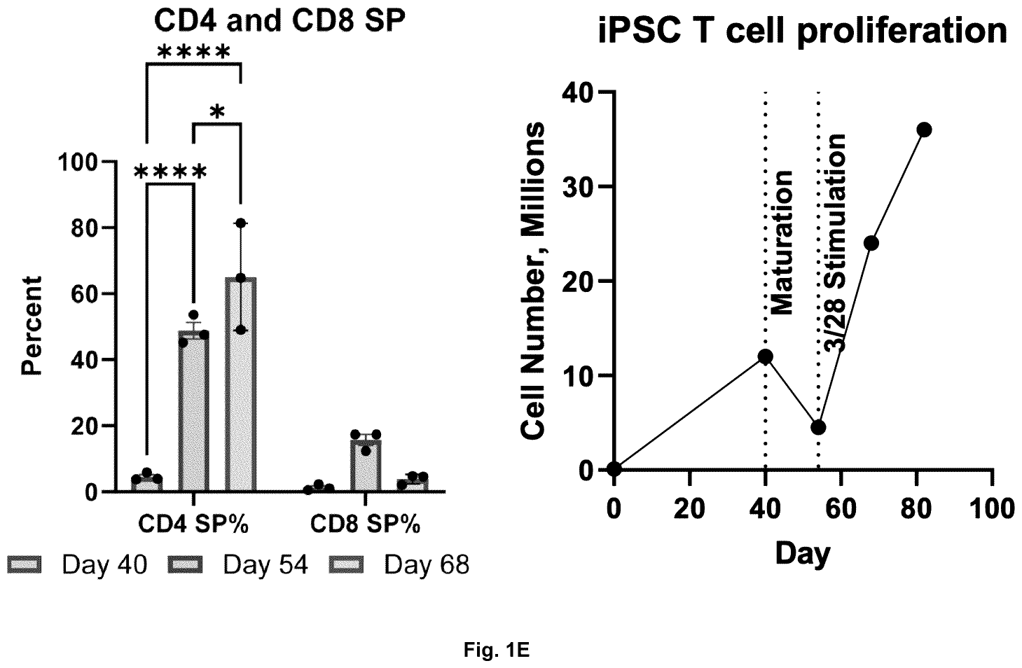

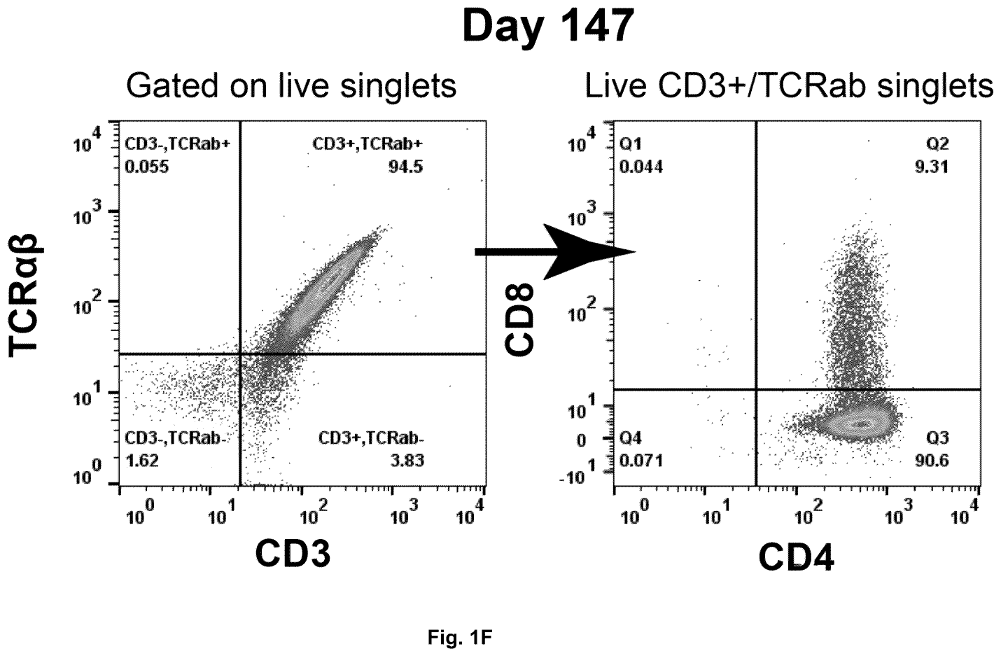

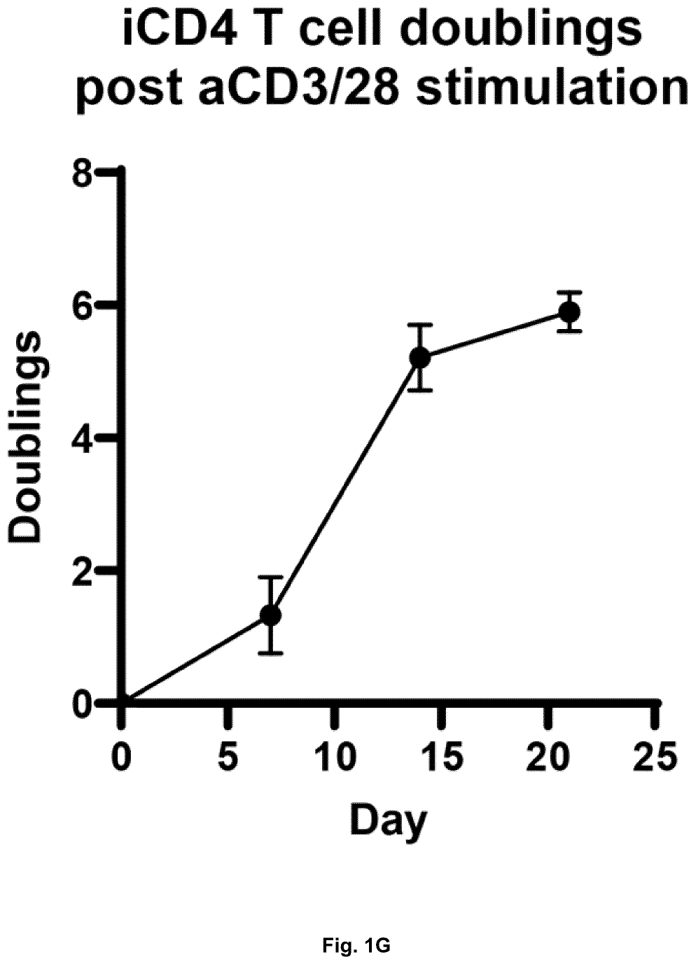

A previously published T cell generation protocol utilized a doxycycline-inducible Notch 1 intracellular domain in the first 3 days of hematopoietic stem and progenitor induction that robustly increases the proportion of cells which can access the T cell lineage (Heinze et al., Stem Cell Reports 17, 2610-2628, 2022). By following this 12-day hematopoietic stem and progenitor protocol CD34+ progenitors are generated (FIG. 1B). These cells are subsequently transferred onto plates coated with Notch ligand, and after a further 28-33 days, large numbers of DP progenitor cells are produced. The cells express markers predicted of lymphoid progenitors, including CD7 (FIG. 1B) at day 26 after T cell specification followed by CD3, T cell receptor, CD4 and CD8 (FIG. 1B) by day 40. Many groups have used a combination of anti-CD3/CD28 in the presence of Notch ligand in combination with IL-7 or IL-15 during maturation of double positive to CD8 single positive cells (Montel-Hagen and Crooks, Experimental Hematology 71, 24-31, 2019; Iriguchi et al., Nature Communications 12, 430, 2021; Heinze et al., 2022). To screen for signals that could allow or block access to the CD4 lineage, elements were systematically removed from the culture, followed by initiation of maturation via stimulation through the T cell receptor. The process began by moving the double positive cells to a coating with no Notch ligands before beginning a DP to SP maturation with IL-7 and anti-CD3/CD28.

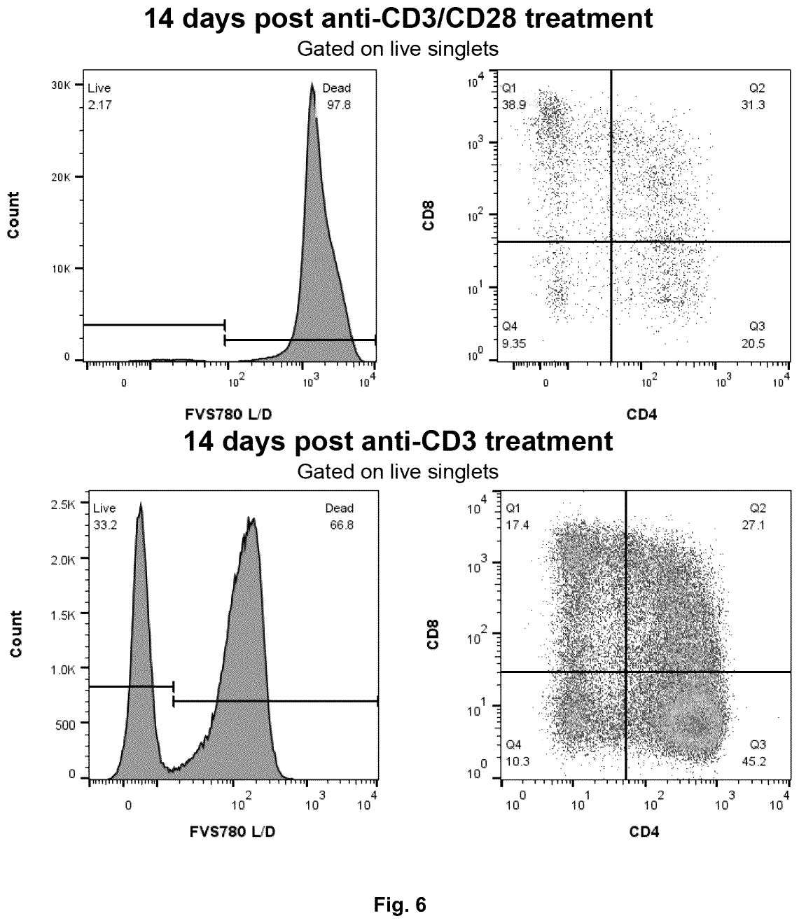

A population of CD4 SP cells emerged from the Notch ligand-free coating condition; however the viability of the differentiated cells was poor (FIG. 6). It was hypothesized that the anti-CD3/CD28 signal was too strong resulting in negative selection and apoptosis. Removal of the co-stimulatory signal by moving to treatment with anti-CD3 alone increased viability of the iT cells after DP to SP maturation (FIG. 1C). 14 days after anti-CD3 and IL7 treatment on Notch-ligand free coating the initially DP cells lose expression of the CD8 coreceptor, maintain expression of the CD4 coreceptor while down regulating CD3 and TCRαβ in response to stimulation (FIG. 1C).

DP to SP specification in the thymus is normally correlated with loss of apoptotic response to TCR stimulation in inflammatory conditions. In order to test the capacity of iCD4 cells to proliferate during stimulation, the newly specified iCD4 cells were treated with anti-CD3/CD28 in the presence of high-dose IL2. The iCD4 cells proliferated effectively in these conditions (FIG. 1D-E), allowing for multiple rounds of stimulation and rest. CD4 expression was maintained throughout this expansion stimulation along with recovery of TCRαβ and CD3, while the cells remained negative for CD8 (FIG. 1D). These iT CD4 cells can be expanded in vitro for long periods of time, while maintaining their T cell identity at least for 140 days, with 2 rounds of stimulation and expansion (FIG. 1F).

iCD4 Cells Map to Developmental Stages Seen in Thymocytes.

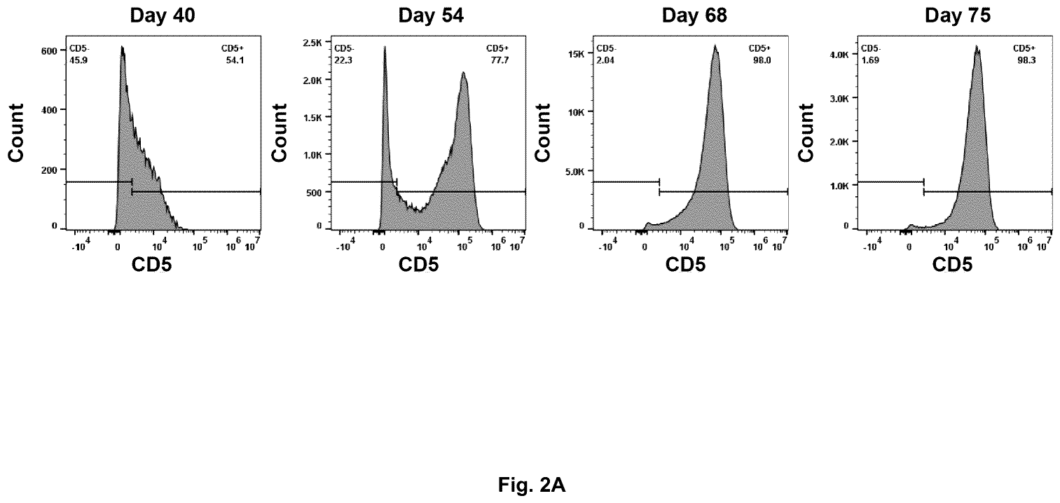

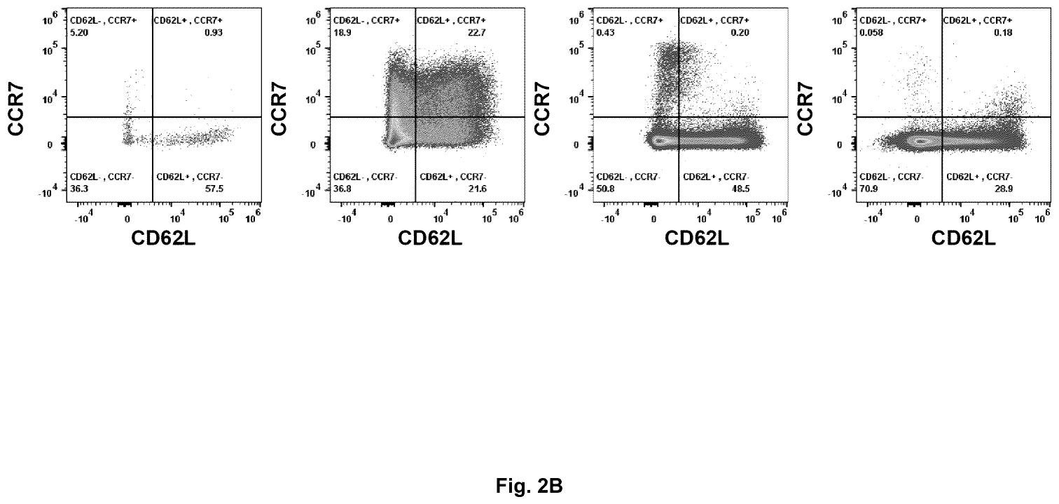

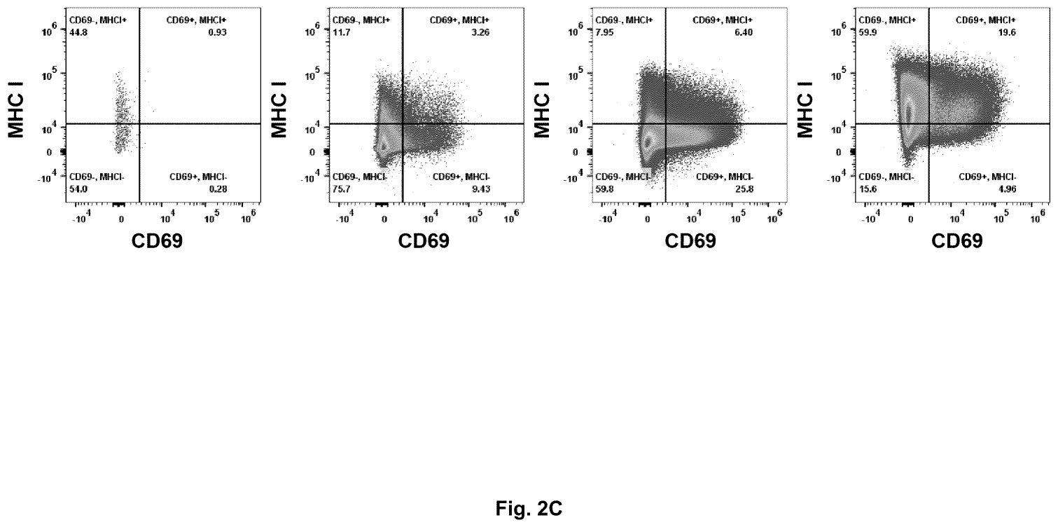

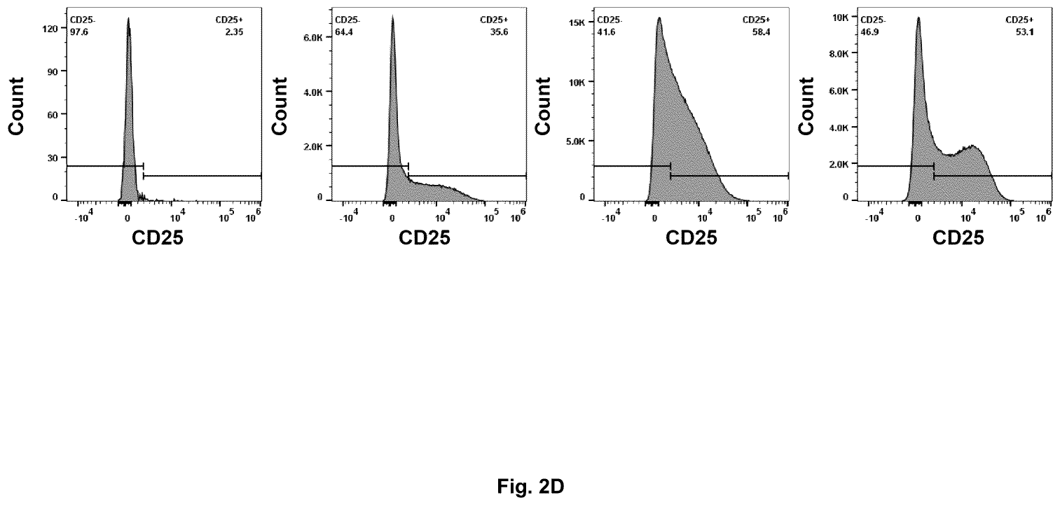

In order to further understand the developmental trajectory of iT CD4 cells, high-parameter flow cytometry was performed. Initially at day 40, after magnetic depletion of TCRγδ positive iT cells, the DP cells expressed CD4 and CD8, along with CD3 and TCRαβ. Treatment with anti-CD3 on Retronectin® coating for 14 days resulted in CD8 downregulation as observed above. Stimulation of the cells induced upregulation of CD5 as marker of T cell maturation (FIG. 2A). After restimulation the cells proliferated and expressed TCRαβ, CD3 and CD4 (FIG. 1C).

CCR7 is upregulated in the process of positive selection and acts to direct cells to the thymic medulla; therefore, the dynamics of CCR7 expression during DP to CD4 SP maturation were examined. Upregulation of CCR7 was observed at day 58 of culture, likely corresponding to positive selection. CD62L (L-Selectin) has been reported to be upregulated in the most mature SP thymocytes as well as mature naive T cells (Fink, Annual Review of Immunology 31, 31-50, 2013). In this system CD62L is expressed early on and became highly expressed in a population of the SP cells upon maturation (FIG. 2B). Further evidence for positive selection of iCD4 cells was obtained through staining for CD69 and MHC class I. CD69− DP cells represent a pre-selection group of DP T cells, while CD69+ cells are early in the lineage commitment process post-selection. CD69, while likely upregulated following the second stimulation of the SP T cells at day 54, is downregulated by day 75 of differentiation (FIG. 2C). Similarly, MHCI initially not expressed in DP cells, was highly expressed by day 75 and its expression correlated well with the acquisition of proliferative capability by the SP iT cells (FIG. 2C).

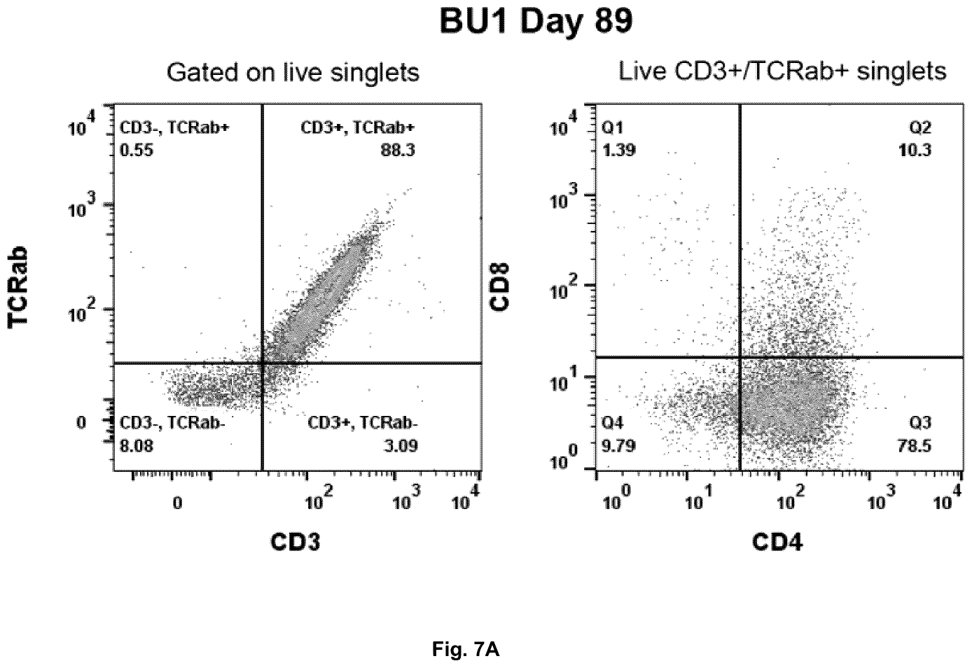

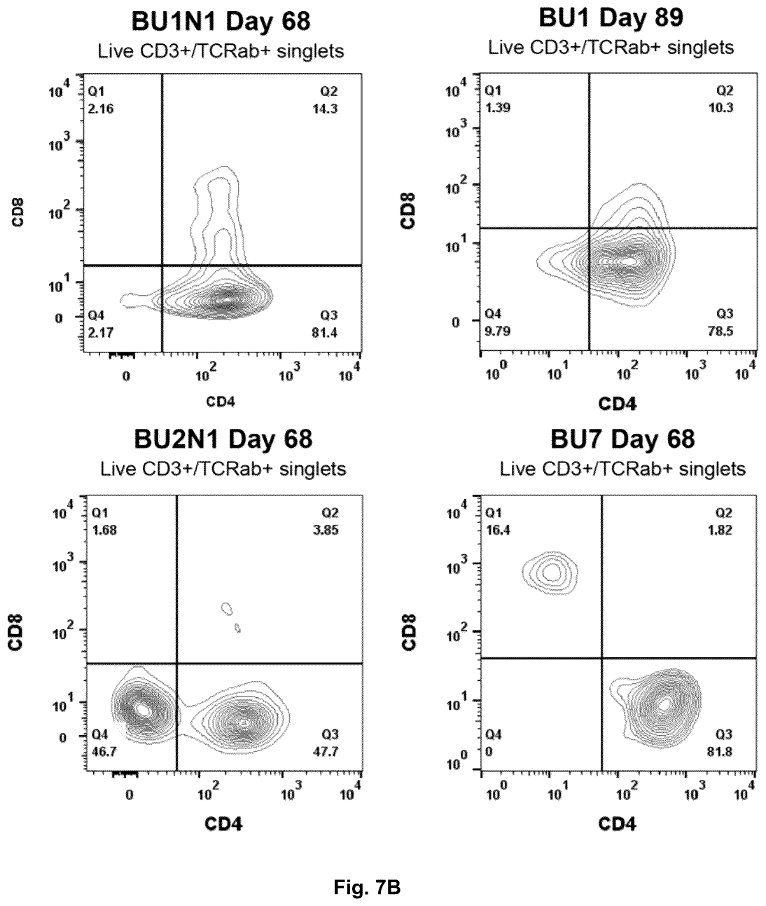

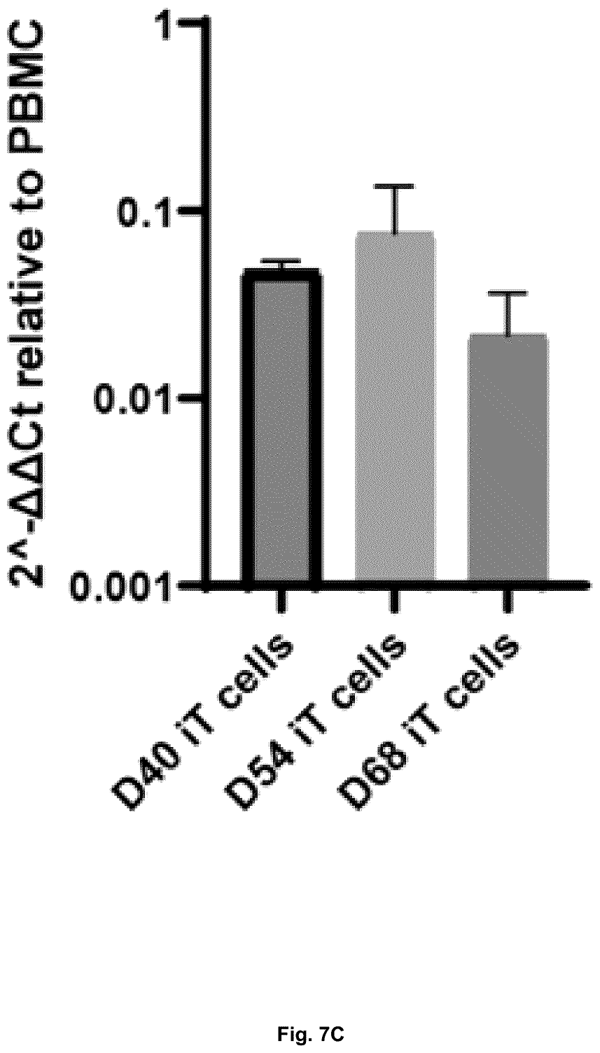

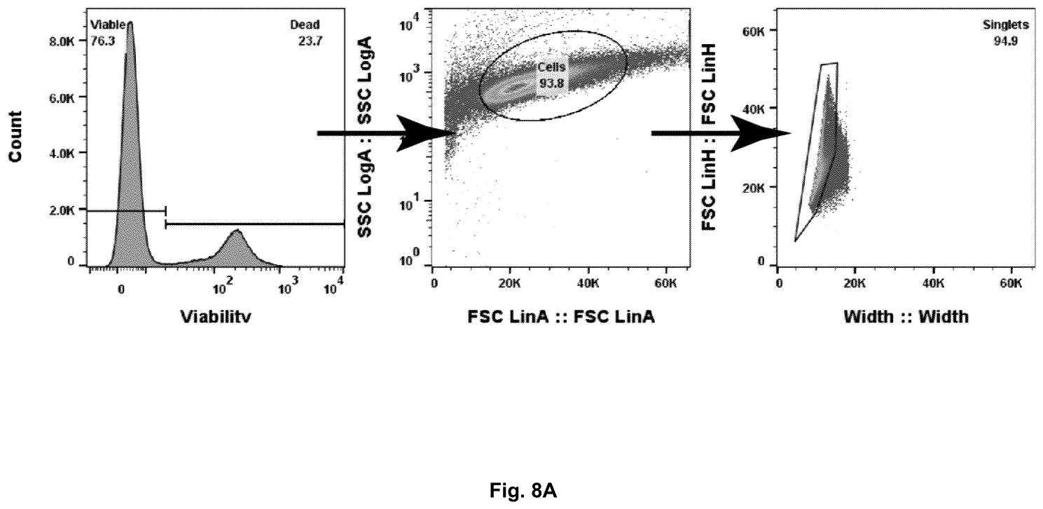

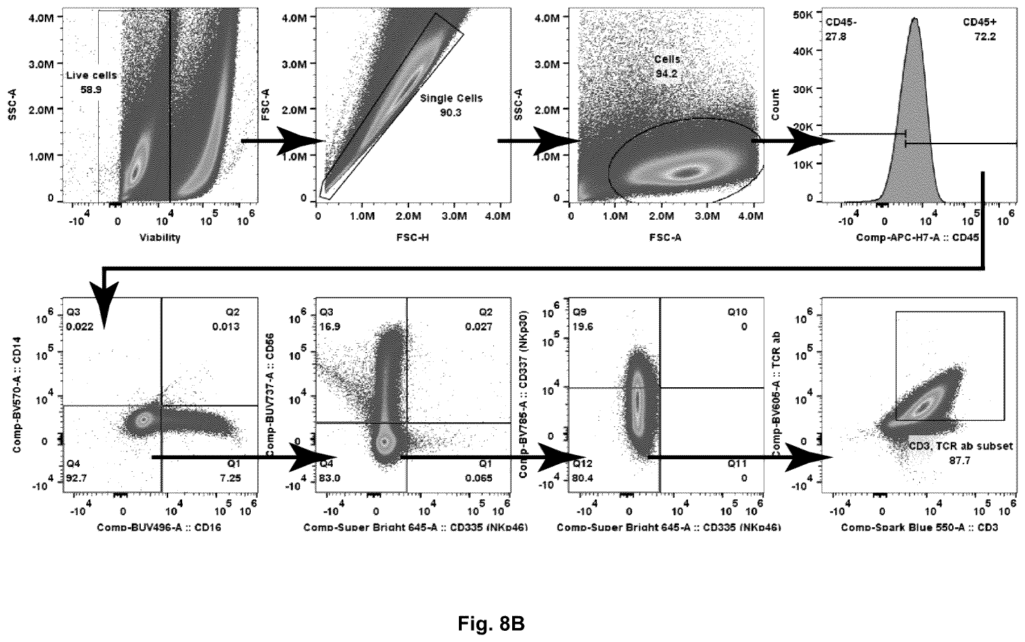

Further evidence of the maturity of iT CD4 cells comes via analysis of the transcription factor ThPOK, (also known as zbtb7b), which is a lineage specific transcription factor of CD4 T cells. During differentiation, transient upregulation of ThPOK in the DP cells transitions into stable expression in mature CD4 cells. Despite lower expression levels than in PBMC, as detected by RT-qPCR, ThPOK was clearly expressed at every timepoint within the iCD4 cells (FIG. 2D). This protocol gives rise to a population of CD25+CD4+ cells, which includes T regulatory cell progenitors (FIG. 7C).

Mature iCD4 Cells Show Proliferation and Classical CD4 Helper Cytokine Production.

The characteristic functional feature of a mature CD4 T cell, in addition to proliferation, is the capability to make and release cytokines in response to TCR stimulation. Conventionally, CD4 T cells are divided into helper subsets, each capable of releasing their own set of potential cytokines. To interrogate the capability of the CD4 cells to produce cytokines, stimulation with phorbol 12-myristate 13-acetate (PMA) and ionomycin was performed. PMA and ionomycin cause the release of the full range of cytokines that a T cell is able to produce.

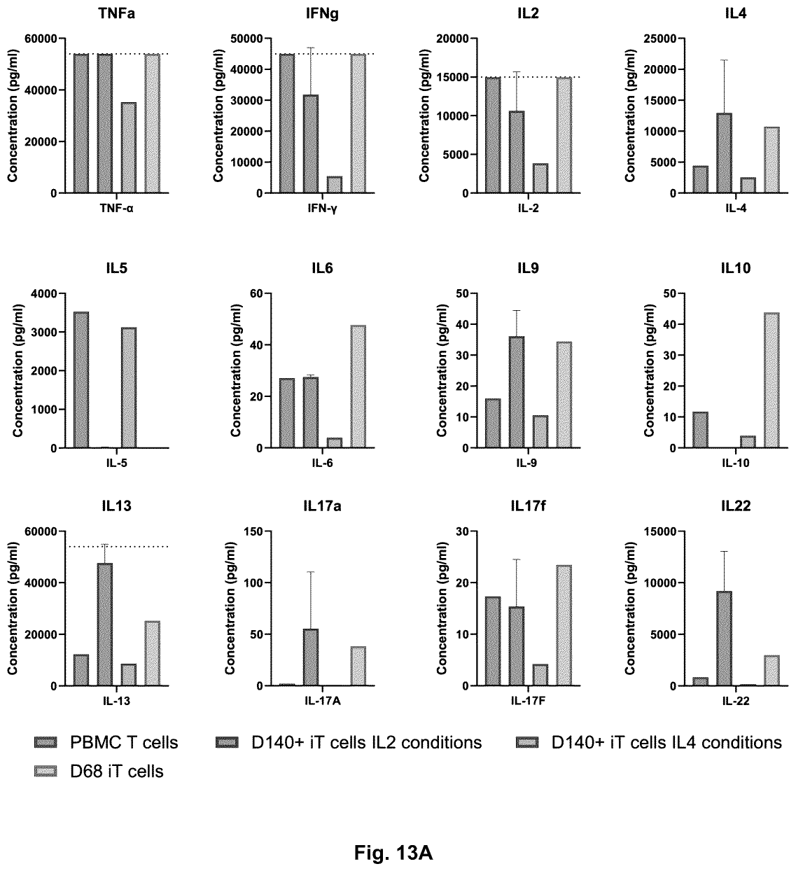

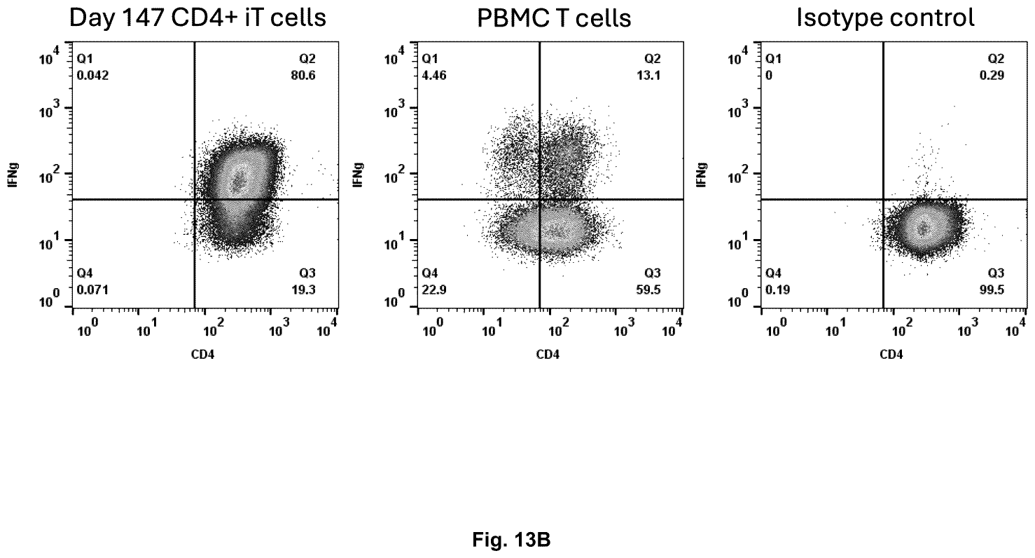

Cells early after specification which have undergone a single round of expansion stimulation (day 68), as well as cells in culture for a long period after specification and underwent 2 or more rounds of expansion (day 140+), were exposed to PMA and ionomycin to induce cytokine release. Further, a group of long-term culture cells was treated with IL2 and high dose IL4 to induce a Th2 program in any remaining naive CD4SP cells. CD4+ iT cells produced a broad variety of cytokines, with robust secretion of the inflammatory Th1 cytokines TNFα and IFNγ along with IL2 (FIG. 13A).

Because supernatant concentration does not differentiate between a few highly prolific cytokine producers and the majority of the iT cells secreting these cytokines, protein trafficking inhibition during PMA/ionomycin stimulation combined with flow cytometry was utilized. This analysis revealed extensive production of IFNγ by the iCD4 cells (FIG. 13B), with comparable levels to primary CD4+ cells. The observed high percent of IFNγ+iCD4 cells reflects Th1 specification due to strong stimulation through the TCR in the presence of high concentration IL2 in long term culture.

Single cell RNA-Sequencing of iCD4 demonstrates a transcriptional profile characteristic of activated CD4 T helper cells.

### DISCUSSION

Described herein is a method of generating functional CD4 SP T cells from induced pluripotent stem cells in a feeder free manner through the removal of Notch signal during TCR mediated DP to SP transition. The resulting cells have both proliferation and cytokine production capabilities in response to stimulation. While it is known that some combination of strength and duration of signal influence T lineage divergence, the downstream effects of Notch ligand withdrawal are currently still in question, particularly in the context of iPSC derived T cells grown in the presence of high concentrations of Notch ligand DLL1 or DLL4. Across multiple iPSC lines withdrawal of Notch ligands allow for access to the CD4 lineage. It was hypothesized that strong signaling of Notch is acting to modulate the strength of signal through the T cell receptor in this system; mechanistic studies can also be performed.

The combination of broad cytokine production and CD62L positivity demonstrate the presence of naive T cells within the cultures. After emigrating from the thymus, T cells continue to develop, spending up to 3 weeks maturing further from a recent thymic emigrant to a naive state and being specified to specific helper subsets. In the iPSC derived CD4 T cell system, the timepoint where naïve or naïve-like cells can be observed can be identified. Treatment of naive cells with various cytokines permits polarization of iT CD4 cells into specific helper subsets and provides a screening platform to reveal specification pathways. T regulatory cells can be induced from naive T cells through treatment with TGFB, IL2, retinoic acid and rapamycin during CD3/CD28 stimulation of naive T cells and can act in a highly anti-inflammatory manner. CD25-positive CD4 cells observed after CD3 stimulation are hypothesized to correspond to CD4+CD25+ T regulatory cell progenitors, and isolation of this subset followed by analysis of CTLA4 and TGFβ expression further defines this CD4+ population.

The artificial thymic organoid (ATO) model uses a mix of Notch ligand expressing feeder cells as well as iPSC derived lymphoid progenitor cells to create an organoid capable of producing CD4+ single positive T cells. The complexity of organoid models shrouds the specific differences in local environment and signaling through the TCR or accessory molecules that allow for access to the CD4 lineage. A key benefit of CD4 generation in a feeder-free context is the increased scalability and control of the final cell product. Because this method does not rely on a second cell type to support the developing T cells, there is no need to purify the final SP cells or maintain and arrest the growth of feeder layer cells. Additionally, iT cells generated by this protocol can be frozen and later thawed at days 12, 26 or any time after day 68 allowing for cell banking.

By highlighting a scalable, highly efficient method for the generation of iPSC derived CD4 cells from iPSC DP cells, this technology permits advanced iPSC derived T cell therapies. CD4 cells are a key element of modern T cell therapies such as CART, and the ability to produce large numbers of Treg cells opens new approaches to the treatment of inflammatory autoimmune disorders.

### Experimental Procedures

### Tissue Culture Conditions and Cell Lines

Human iPSC lines were maintained on 6-well tissue culture treated plates coated with hESC-qualified Matrigel™ in 2 mL of mTeSR+ media with added Primocin™. Media was changed daily, excepting weekends, where cells were fed twice the standard amount of mTeSR. Cells were passaged weekly using ReLeSR according to manufacturer protocols. Cells were grown at 37° C., 5% CO₂ in standard incubators. iPSC lines used in this study were BU1c2 (XY, EF1a-hSTEMCCA4 loxp lentiviral infection, Cre-excised), BU1c2 TetOn:NICD1, and BU2-15-Cr10 TetOn:NICD1 (XY, EF1a-hSTEMCCA4 loxp lentiviral infection, Cre-excised). Generation of TetOn:NICD1 lines outlined in prior work (Heinze et al., 2022). Human PBMC cells were isolated by density gradient centrifugation with FICOLL and maintained in X-Vivo® 15 with 50 IU IL2 added in T75 or larger flasks.

Differentiation from iPSC to Double Positive T Cells

iPSC cells are differentiated to hematopoietic stem and progenitor cells as previously described over a 12-day period (Heinze et al., 2022). CD34+ progenitors are isolated from all floating cells by MACS separation using MILTENYI CD34+ selection kit and LS columns (MILTENYI). CD34+ progenitors are seeded at 5*10⁴ onto TC untreated plates coated with Stemspan™ Lymphoid Differentiation Coating Material as per manufacturer instructions (STEMCELL TECHNOLOGIES). The progenitors are fed with Stemspan™ SFEM II media (STEMCELL TECHNOLOGIES) supplemented with Stemspan™ Lymphoid Progenitor Expansion Supplement (STEMCELL TECHNOLOGIES) and Primocin™. After initial seeding in 1 mL of expansion media cells are cultured for 3 days (Day 15), then fed with an additional 1 mL of expansion media. At Day 19 half of the media is aspirated from each well and replaced with fresh media, then cells are moved to a plate with new coating. Half media changes are performed until day 26, when cells are harvested and re-seeded at 0.5 to 1*10\hat{ }6 cells per mL on new plates coated with Stemspan™ Lymphoid Differentiation Coating Material in Stemspan™ SFEM II media supplemented with Lymphoid Progenitor Maturation Supplement (STEMCELL TECHNOLOGIES) and Primocin™. After 3 days maturation media was added as with expansion culture. Half media changes are then performed with maturation media every 3 or 4 days until day 40, when double positive cells were harvested.

Differentiation from DP T Cells to CD4 SP T Cells and SP T Cell Culture Conditions

DP cells were plated on 12 well untreated plates coated with 10 μg of RetroNectin® (TAKARA BIO) at variable density from 1*10\hat{ }5 to 1*10\hat{ }6 cells per mL in Stemspan™ SFEM II media supplemented with Lymphoid Progenitor Maturation Supplement (STEMCELL TECHNOLOGIES) and Primocin™. At the time of plating media is supplemented with an additional 10 ng/mL of IL7 and 5 μg/mL of purified anti-CD3 antibody clone OKT3. The media is then changed as outlined above with half media changes of Stemspan™ SFEM II media supplemented with Lymphoid Progenitor Maturation Supplement (STEMCELL TECHNOLOGIES), 10 ng/mL of IL7 and Primocin™. At day 54 (14 days after initiation of DP to SP transition) SP T cells can be harvested.

SP iCD4 T cells are moved to a freshly coated RetroNectin® plate, in Stemspan™ SFEM II media supplemented with Lymphoid Progenitor Maturation Supplement (STEMCELL TECHNOLOGIES), 200 IU IL2 and Primocin™. Expansion is initiated with the addition of 25 μg/mL of anti CD3/CD28 Immunocult™ human T cell activator (STEMCELL TECHNOLOGIES). Through the expansion process cells are fed with half media changes as described above.

At day 68 iT cells begin to be adapted to X-Vivo® 15 media (LONZA) supplemented with 200 IU IL2 by performing half media changes with X-Vivo® 15 media.

### Flow Cytometry

Single cell suspensions were harvested from cultures and resuspended in PBS. Where a live/dead dye was used the cells are incubated with fixable viability stain 780 (BD BIOSCIENCES) at 1:1000 for 30 minutes at room temperature in the dark. Cells were then washed with FACS buffer (2% fetal bovine serum in PBS) and a master mix consisting of antibodies detailed in the materials section. Samples were incubated for 30 minutes at room temperature in the dark. All antibodies were used at 1:100 concentration. Stained samples were read on a STRATEDIGM instrument equipped with 405, 488, 552 and 640 nm lasers. Analysis performed in FLOWJO software.

### Spectral Flow Cytometry

Samples were prepared and stained as outlined in flow cytometry. Master mix contained 1× human Fc block (BD BIOSCIENCES) and 1× BRILLIANT Stain Buffer (BD BIOSCIENCES) following manufacturer instructions. All antibodies were titrated and used at optimal concentration to minimize background staining while retaining signal. Samples were read on a 6 laser CYTEK AURORA instrument and unmixed on the instrument. Analysis performed in FLOWJO software.

### Cytokine Quantification

T cell samples were seeded at 1 million cells per mL in X-Vivo® 15 media (LONZA) which had not been supplemented with any cytokines. Phorbol 12-myristate 13-acetate and ionomycin were added at 25 ng/mL and 1 μg/mL concentrations respectively. After a 6 hour incubation each well was centrifuged and supernatants were harvested and stored at −80 degrees Celsius.

In one instance the cells were also treated with protein transport inhibitor Brefeldin A, and then stained for flow cytometry using EBIOSCIENCES FoxP3/transcription factor staining kit according to manufacturer instructions.

To perform multiplexed analysis of cytokines, a LEGENDplex™ HU Th Cytokine Panel (12-plex) w/FP V02 kit from BIOLEGEND was used. Supernatants were thawed on ice, then diluted in 2 parts assay buffer for each part supernatant. Manufacturer instructions were followed to prepare samples, which were then read on a 6-laser CYTEK AURORA. Data was then analyzed in LEGENDPLEX software (BIOLEGEND).

### FIGURES

FIGS. 1A-1B: Analysis of cells from throughout the process of generating and maintaining iCD4+ T cells. (FIG. 1A) Schematic of iT differentiation to CD4 cells. (FIG. 1B) Representative flow cytometry of key developmental timepoints and metrics of DP T cells. (FIG. 1C) Representative flow cytometry of D54 T cells. (FIG. 1D) Representative flow cytometry of D68 T cells post expansion. (FIG. 1E) Percentage of CD4 and CD8 positive T cell from iCD4 differentiation by flow cytometry, including at day 54 and 68, N=3 and N=2 respectively. Number of cells, starting with initial iPSC seeding. Dotted line indicates timepoints of stimulation of T cells. (FIG. 1F) Representative flow cytometry of T cells from long term culture. Parent gating for each plot as indicated.

FIGS. 2A-2D: Flow cytometry of multiple timepoints of specification (day 40 and day 54) and expansion (day 68 and day 75) of iT cells. The populations shown are gated on Live/Single/CD45+/CD14−/CD16−/CD56−/CD335−/CD337−/CD3+/TCRαβ+ cells, with the exception of Day 54, which is gated on Live/Single/CD45+/CD14−/CD16−/CD56−/CD335−/CD337− cells due to poor CD3 and TCRαβ expression. (FIG. 2A) Flow cytometry showing expression of CD5. (FIG. 2B) Flow cytometry showing expression of CD62L and CCR7. (FIG. 2C) Flow cytometry showing expression of CD69 and MHC I. (FIG. 2D) RT-qPCR of ThPOK, Δ ΔCT relative to PBMC using ACTB for the housekeeping gene.

FIG. 7C: qPCR analysis of ThPOK transcription over 1 differentiation of BU1N1 cells at day 40, 54 and 68, relative to PBMC T cells using ACTb as a housekeeping gene in technical triplicate. Mean+SEM.

FIGS. 13A-13B: Analysis of cytokine secretion by iCD4+ T cells. (FIG. 13A) Concentration of cytokines in the supernatant of media after PMA/Ionomycin stimulation of T cells at 1*10\hat{ }6 cells/mL in picograms/mL. Dotted line indicates the upper limit of detection for the cytokine. N varies from 1 to 3 per group. (FIG. 13B) Flow cytometry of PBMC and Day 140+ iT cells for CD4 and intracellular interferon γ, pre-gated on live cells.

### Example 2: Notch Withdrawal is Key for Robust Differentiation of CD4 Helper T Cells from Human iPSC

It has not previously been possible to differentiate CD4 T cells from induced pluripotent stem cells. Described herein is a differentiation platform that allows for CD4 development. The iCD4 cells are then characterized and tested for their proliferation and cytokine production capability.

The human T cell immune system relies on both CD8+ cytotoxic T cells and CD4+ helper T cells to function. CD8 T cells act to kill when activated through their T cell receptor (TCR), while CD4 T cells generate and release cytokines which act to modulate the effect of T cells and other immune subsets. Modern induced pluripotent stem cell (iPSC) models use developmental cues to bring the cells from a pluripotent state through development ending in mature differentiated state. However, current iPSC to T cell differentiations fail to generate CD4+ T cells.

FIGS. 1A-1D: Analysis of cells from throughout the process of generating and maintaining iCD4+ T cells. (FIG. 1A) Schematic of iT differentiation to CD4 cells. (FIG. 1B) Representative flow cytometry of key developmental timepoints and metrics of DP T cells. (FIG. 1C) Representative flow cytometry of D54 T cells. (FIG. 1D) Representative flow cytometry of D68 T cells post expansion.

FIGS. 2A-2D: Flow cytometry of iT cells. The populations shown are gated on Live/Single/CD45+/CD14−/CD16−/CD56−/CD335−/CD337−/CD3+/TCRαβ+ cells, with the exception of Day 54, which is not gated on CD3 and TCRαβ+ cells due to poor expression. (FIG. 2A) CD5 staining. (FIG. 2B) CD62L and CCR7 staining.

FIG. 7C: ThPOK transcription factor expression by RT-qPCR. Δ ΔCT relative to PBMC using ACTB for the housekeeping gene.

FIGS. 13A-B: Concentration of cytokines in supernatant after PMA and Ionomycin stimulation of T cells at 1*10\hat{ }6 cells/mL in picograms/mL. Dotted line indicates upper limit of detection. N varies from 1 to 3.

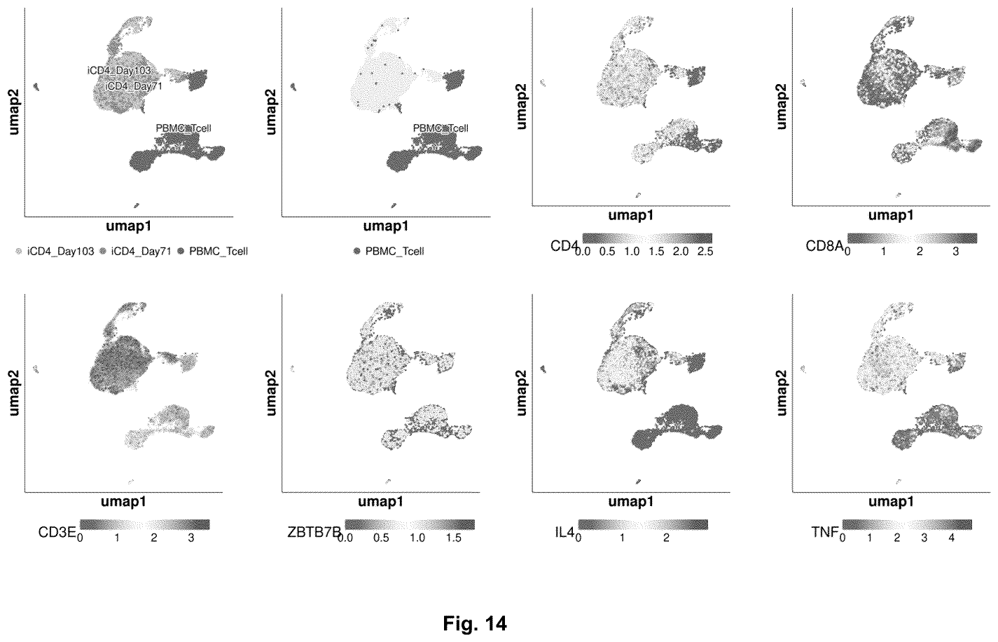

FIG. 14: Single cell RNA sequencing UMAP of PBMC, D71 and D103 iT cells showing selected genes

CD4 cells generated through this method express expected surface markers while acquiring functional features of peripheral CD4 T cells in cytokine release and proliferation.

### Example 3: Generation of Effector CD4+ T Cells from Human iPSC

Off the shelf CD4+ T cell therapies, particularly those with immunoregulatory or cell repair functions, can be transformative for the CAR therapies for cancers and treatment of chronic inflammatory diseases. However, progress is stunted in this area due to challenges generating human CD4+ T cells from induced pluripotent stem cells. Described herein is a key role for the withdrawal of Notch ligand during the final step of stimulation through the T cell receptor to prompt T cell maturation allowing access to the CD4 lineage in iPSC T cells (iCD4+ T cells). Functional analyses of iCD4+ T cells using a high-parameter CyTOF intracellular cytokine staining panel revealed both canonical Th1 cytokine signatures and cells producing varying combinations of other cytokines including IL-4, IL-8, and IL-13. Single cell RNA sequencing of iCD4+ T cells demonstrated a transcriptional signature similar to human blood CD4+ T cells. This robust yet simple platform represents allows for the generation of off the shelf iCD4+ T cell therapies with utility for the treatment of a panoply of diseases including cancer and inflammatory autoimmune disorders.

As described herein, Notch ligand withdrawal during iPSC derived T cell maturation allows access to the CD4+ lineage, with robust generation of long-lived iPSC CD4+ Helper T cells expressing characteristic genes and markers, and producing high levels of functional cytokines.

### Notch Withdrawal Allows for CD4+ Lineage Emergence.

A previously published T cell generation protocol utilized a doxycycline-inducible Notch 1 intracellular domain in the first three days of hematopoietic stem and progenitor induction that robustly increases the proportion of cells which can access the T cell lineage (Heinze et al., 2022). This 12-day hematopoietic stem and progenitor protocol allows robust generation of CD34+ progenitors (FIG. 1B, left panel). These cells are subsequently transferred onto plates coated with Notch ligand, and after a further 28-33 days, large numbers of DP progenitor cells are generated (FIG. 1B, right panel). These cells express hallmark lymphoid progenitor markers, such as CD7 (FIG. 1B, center panel) at day 26 after T cell specification followed by CD3, T cell receptor, CD4 and CD8 (FIG. 1B, right panels) by day 40. Many groups have used a combination of anti-CD3/CD28 in the presence of Notch ligand with IL-7 or IL-15 during maturation of DP to CD8 SP T cells (Montel-Hagen and Crooks 2019; Iriguchi et al., 2021; Heinze et al., 2022). To identify signals that could allow or block access to the CD4+ lineage, individual factors were systematically removed from the DP to SP maturation culture conditions. When the double positive cells were transferred to RetroNectin® coated wells, a fibronectin derivative lacking Notch ligands, and media supplemented with IL-7 and anti-CD3/CD28, a population of CD4 SP cells emerged, albeit with poor viability (FIG. 6). The anti-CD3/CD28 stimulation was determined to be too strong resulting in negative selection and apoptosis. Removal of the anti-CD28 co-stimulatory signal and stimulation of the cells with anti-CD3 antibody alone dramatically increased the viability of the iT cells after the DP to SP maturation while robustly differentiating into the CD4+ T cell lineage (FIG. 1C) (FIG. 6). As expected in developing T cells, CD3 and TCRab were downregulated in response to stimulation at day 54 (FIG. 1C).

DP to SP specification in the thymus is normally correlated with loss of apoptotic responses to strong TCR stimulation. In order to test the capacity of iCD4+ cells to survive strong TCR stimulation, the newly specified iCD4+ cells were treated with anti-CD3/CD28 in the presence of high-dose IL-2 (200 U/mL). CD4 expression was maintained throughout this expansion stimulation along with recovery of TCRαβ and CD3 expression while remaining negative for CD8 at day 68 (FIG. 1D, 1E). The iPSC-derived CD4+ T cells (iCD4+ T cells) were shown to be expandable extensively in vitro while maintaining their T cell identity for at least 147 days, following two rounds of stimulation and expansion (FIG. 1F, 1G). Treatment with anti-CD3/CD28 in serum free 200 U/mL IL2 conditions resulted in approximately six doublings (FIG. 1G), in line with previously reported T cell proliferation rates in the same media (Mangelinck et al., Frontiers in Immunology Volume 14-2023, 2024). Additionally, iT cells generated by this protocol can be frozen and later thawed at days 12, 26 or any time after day 68 allowing for cell banking.

iCD4+ Cells Map to Thymocyte Developmental Stages.

In order to further understand the developmental trajectory of iCD4+ T cells, multi-parameter flow cytometry covering 23 developmental and functional markers (Table 2) was performed. Following the differentiation protocol outlined in FIG. 1A, developing iT cells were analyzed at day 40, 54, 68 and 75 of differentiation. As expected, most of the DP cells at day 40 were negative for or weakly expressed many of the maturation markers, including CD5, CCR7, CD69, MHC I and CD25 (FIG. 2).

Maturation of the cells induced strong upregulation of CD5 throughout the course of CD4 specification, with a substantial population of CD5hi cells emerging by day 54 while nearly all cells become CD5hi by day 68 and day 75 (FIG. 2A). CD5 intensity has been shown to be reflective of TCR stimulation during selection.

CCR7 is upregulated during primary T cell development within positive selection and directs cells to the thymic medulla. Upregulation of CCR7 was observed on iT cells at day 54 of culture corresponding to a post-selection timepoint. CCR7 expression decreased by day 68 and 75, reflecting restimulation of the TCR and CD28 triggering further specification toward an effector phenotype (FIG. 2B). CD62L (L-Selectin) is upregulated in the most mature SP thymocytes as well as mature naive T cells. In this system, CD62L is expressed early on and became highly expressed in a population of the SP cells upon maturation at day 54. Unlike CCR7 however, CD62L expression is maintained through day 75 in a substantial population of the iCD4+ T cells (FIG. 2B).

Further evidence for positive selection in iCD4+ T cells is demonstrated through CD69 and MHC class I expression. CD69− DP cells represent a pre-selection group of DP T cells, while CD69+ cells appear in the lineage commitment process post-selection. CD69 is upregulated following the second stimulation of the SP T cells at day 54, but downregulated by day 75 of differentiation (FIG. 2C). Similarly MHC-I, initially not expressed in DP cells, becomes highly expressed by day 75 and its expression correlated well with the acquisition of proliferative capability by the SP iT cells (FIG. 2C). CD25 expression, which is negative at day 40, increases throughout the differentiation (FIG. 2D). CD25 expression reflects activation and indicates capacity for IL-2 responsiveness in iT CD4+ cells, as has been shown in primary effector CD4+ T cells.

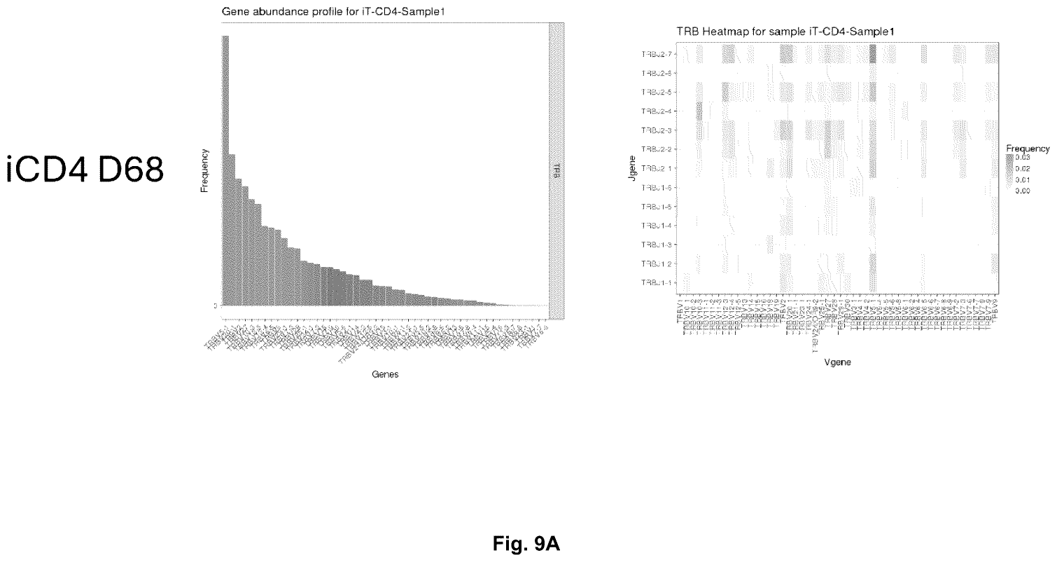

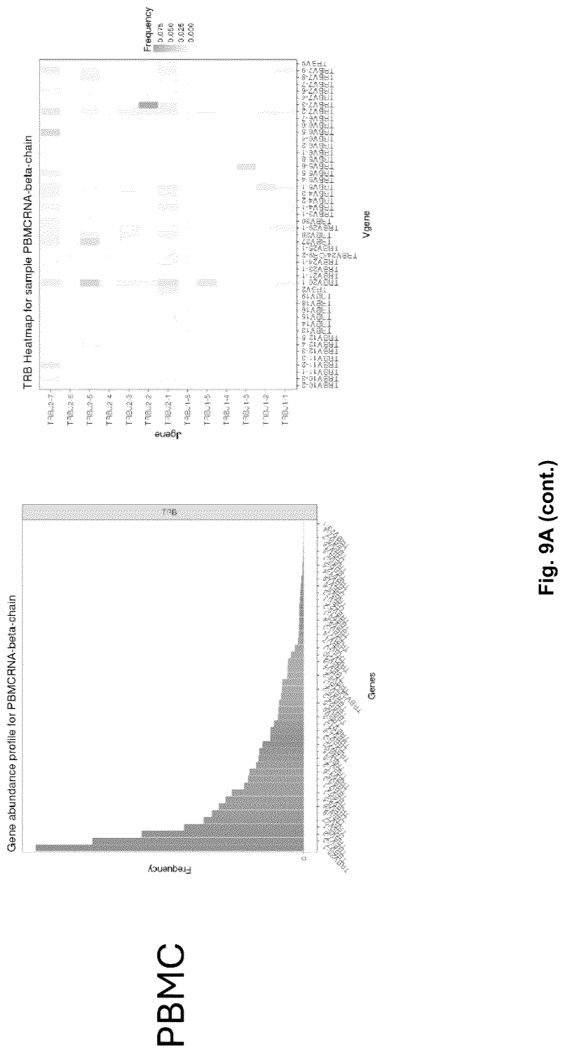

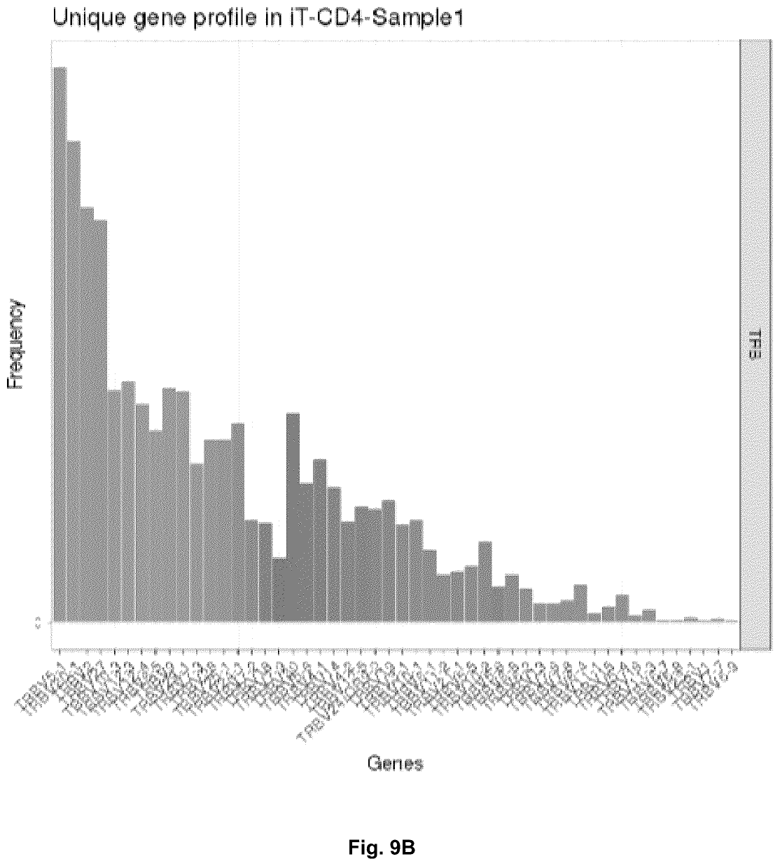

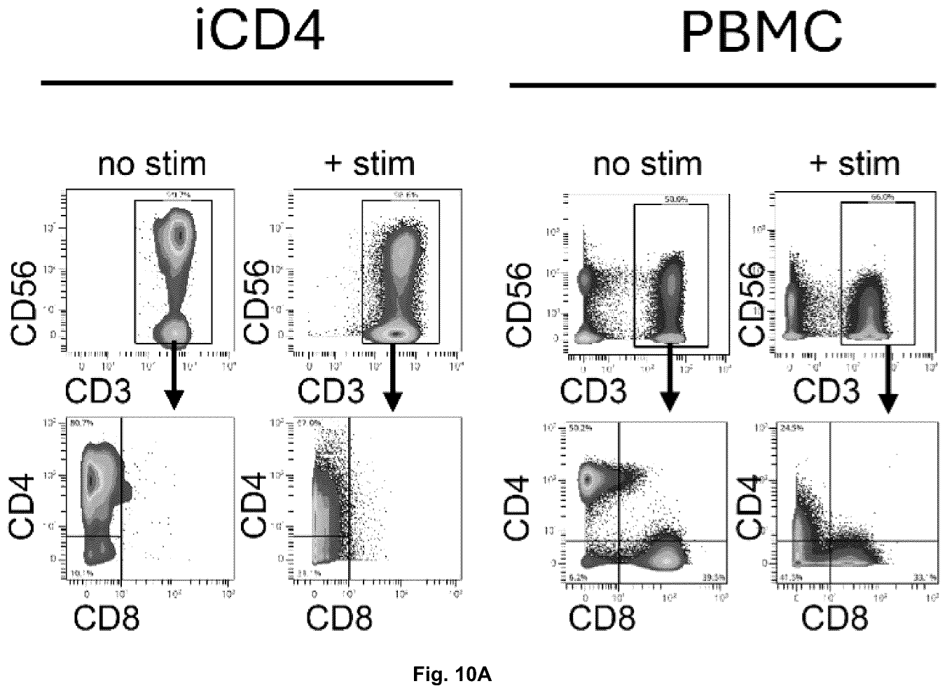

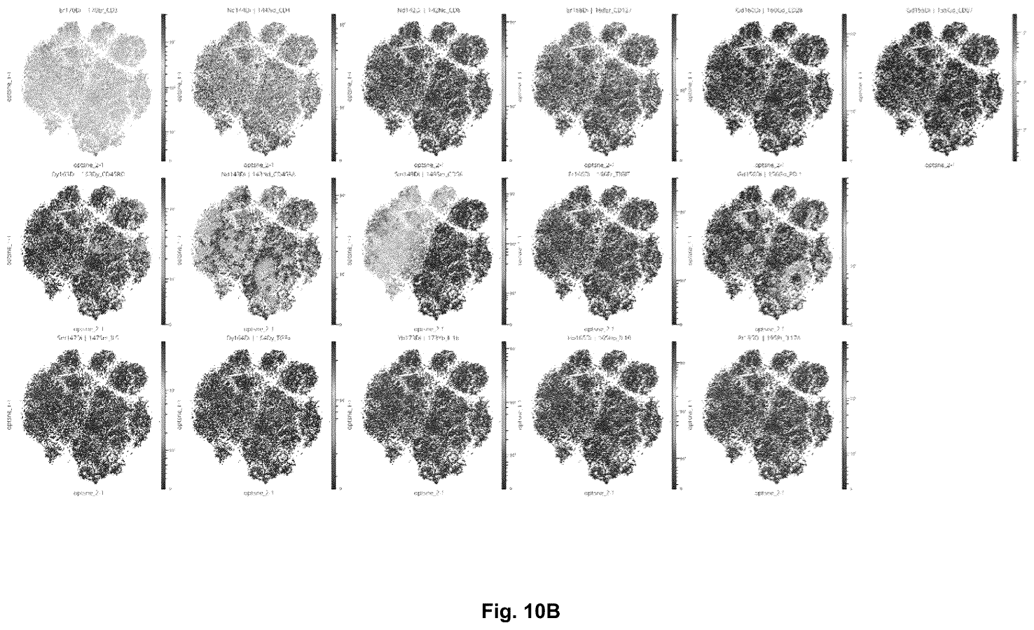

To confirm the identity of iCD4+ T cells, expression of the transcription factor ThPOK (also known as ZBTB7B), a key CD4+ T cell transcription factor (Luckey et al., 2014), was analyzed. Transient upregulation of ThPOK in DP T cells transitions into stable expression in mature CD4+ T cells (Park et al., 2010). RT-qPCR analysis demonstrated that iCD4+ T cells express ThPOK at every timepoint albeit at lower expression levels compared to PBMC (FIG. 7C). Additionally, iCD4+ T cells possess diverse TCRβ V and J usage, indicative of multiclonal iCD4+ T generation (FIG. 9), providing further evidence that this protocol recapitulates normal T cell development.

Cytokine Analysis of iCD4+ T Cells at Single Cell Resolution Reveals Hallmark Th1 Cells and Other T Helper Expression Signatures.

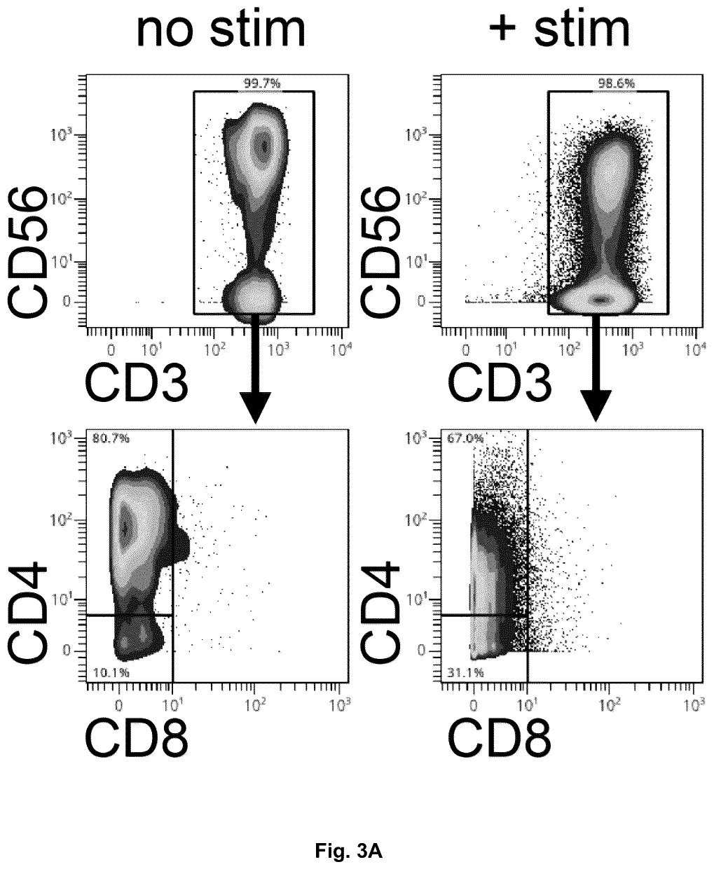

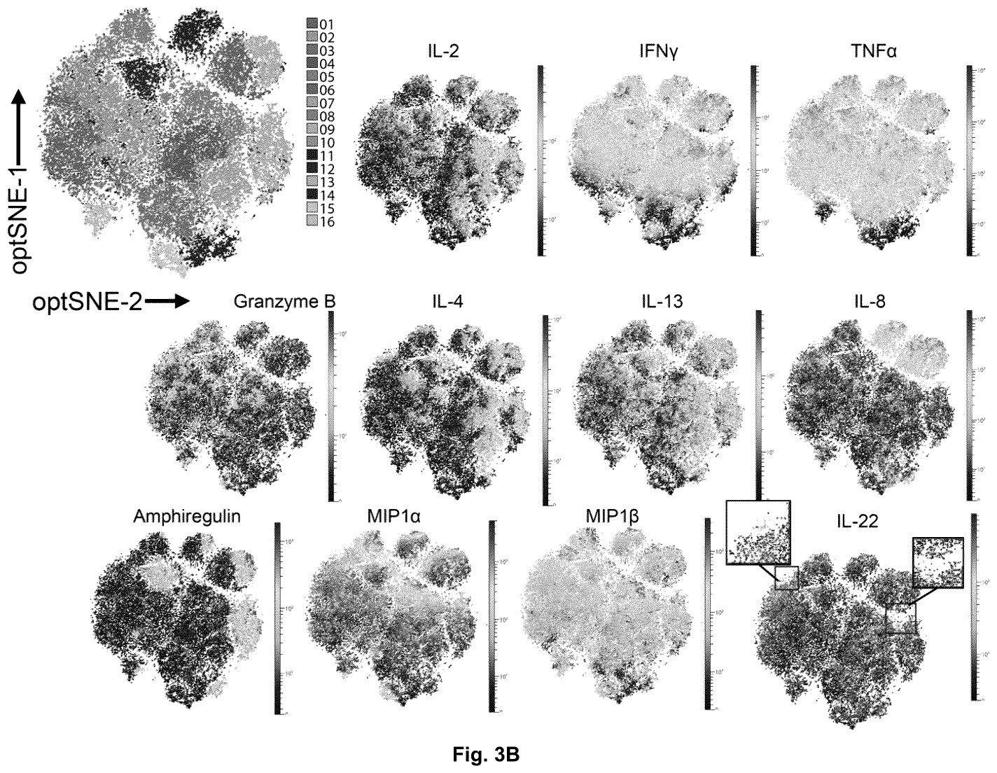

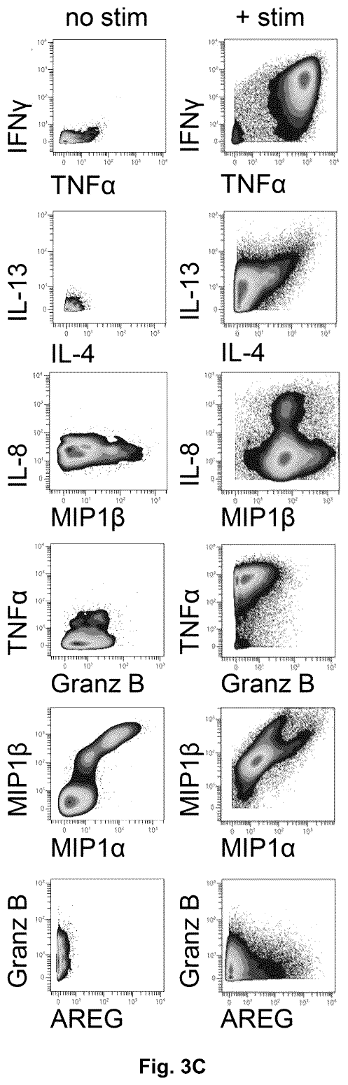

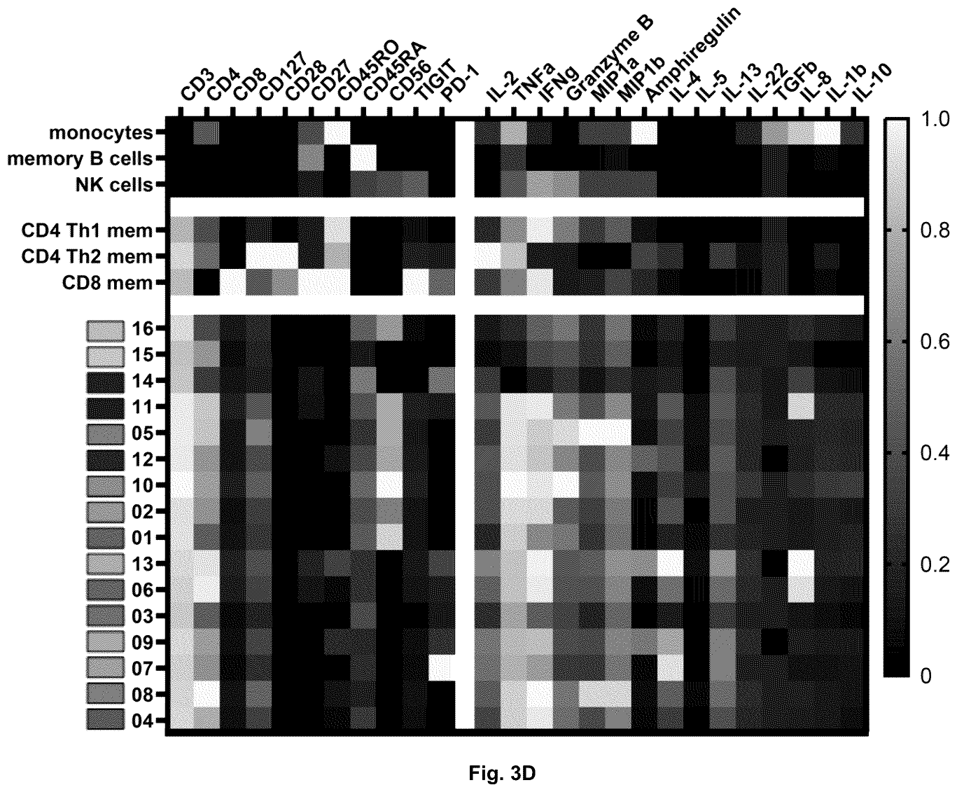

The phenotypic and functional signatures of the day 68 iCD4+ T cells were determined using a 30+ metal intracellular cytokine CyTOF panel (Table 3). After short-term (overnight) culture with either media alone or with phorbol 12-myristate 13-acetate (PMA) and ionomycin, iCD4+ T cells were stained and run on the CyTOF. T cell lineage markers showed all cells were T cells (CD3+), with >90% CD4 single positive, and a negligible percentage of cells expressing CD8 (FIG. 3A, S5). Collected data was visualized into 2D space using opt-SNE and clustered via PHENOGRAPH from expression of both surface and intracellular markers. The vast majority of iCD4+ T cells produced both IFN-γ and TNF-α (FIG. 3B, 3C); this finding aligned with analysis of CD4+ T cells from PBMC (FIG. 3D) and CAR-T populations. Inclusion of additional cytokine readouts revealed diversity of effector function profiles within this ‘Th1’ iCD4+ T cell population, including cells producing both Th2 cytokines IL-4 and IL-13, a notable proportion producing IL-8, co-expression of MIP-1α, and MIP-1b, and five clusters containing strong producers of the cell repair cytokine amphiregulin in a stimulation-specific manner (FIG. 3B, 3C, 3D).

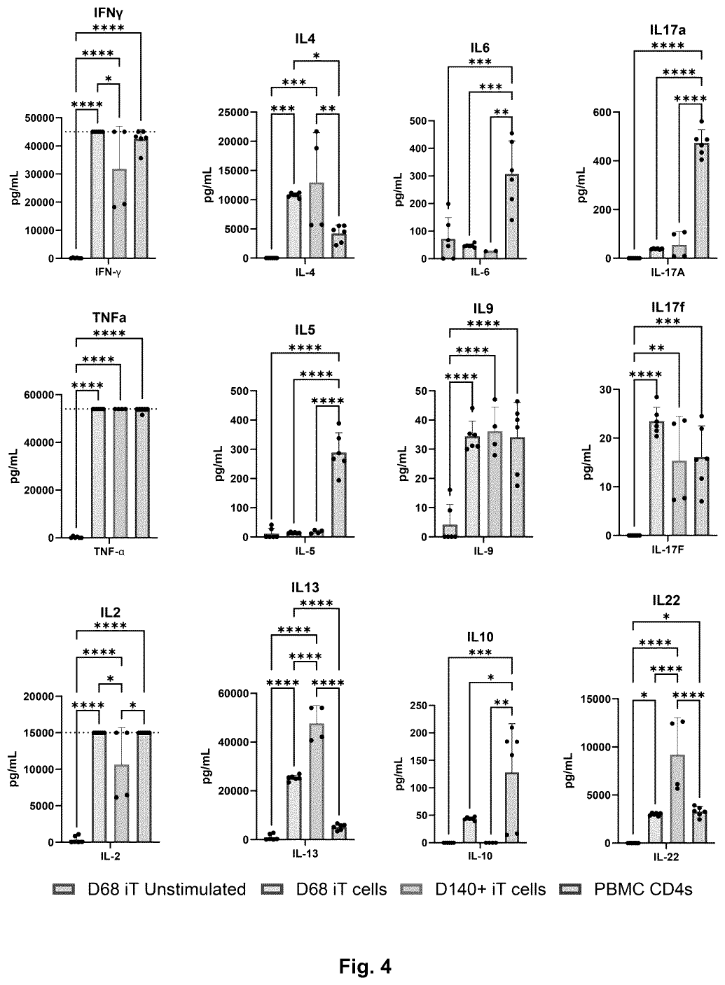

Secretion of cytokines in response to stimulation is key for CD4+ T cell function. Accordingly, the secretion of cytokines into supernatant was measured after six hours of PMA and ionomycin stimulation from iCD4+ T cells that had undergone a single round of expansion stimulation (day 68) and two or more rounds of expansion (day 140+) (FIG. 4). In line with CyTOF findings, the iCD4+ T cells secreted a similar diversity of cytokines, with inflammatory cytokines detected at the highest level (FIG. 4). iCD4+ T cells at baseline without stimulation showed minimal cytokine production and responded upon stimulation with robust cytokine release (FIG. 4).

Bona Fide CD4+ Helper Identity of iCD4+ T Cells Confirmed Via Single-Cell RNA Sequencing.

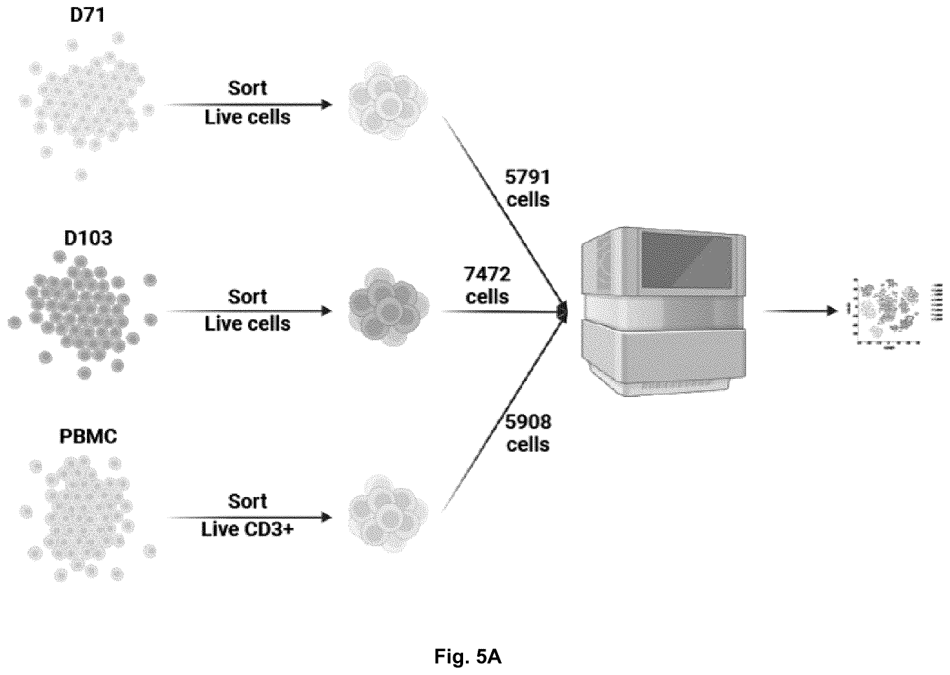

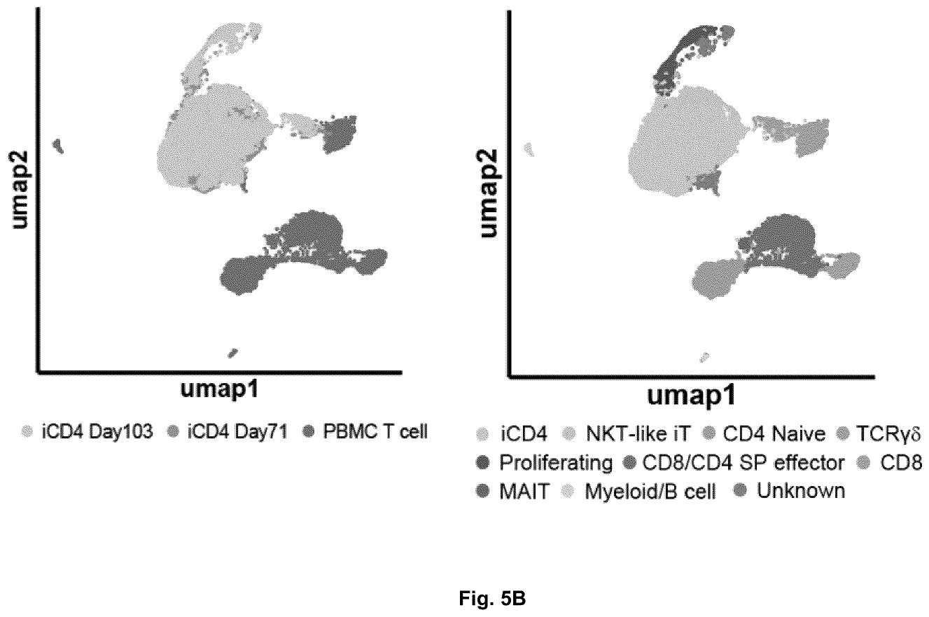

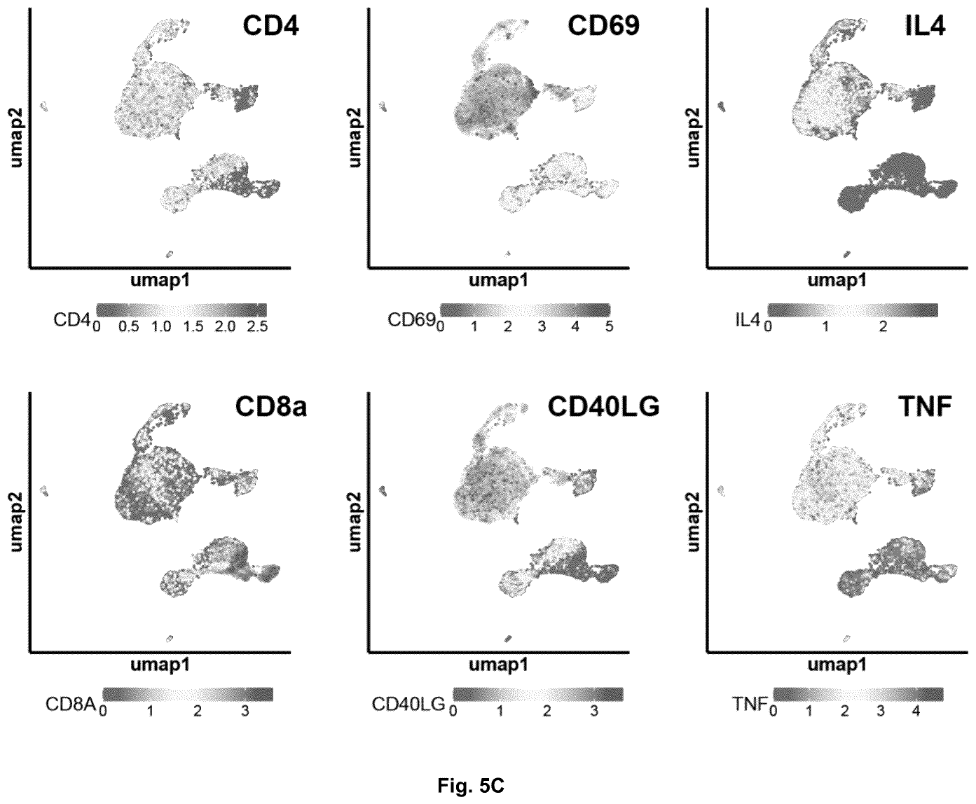

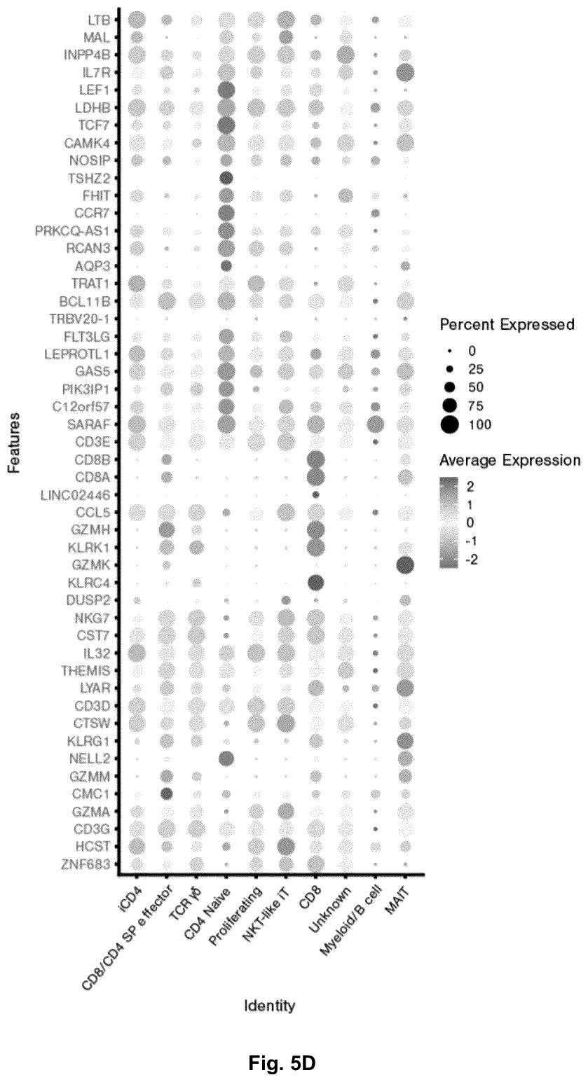

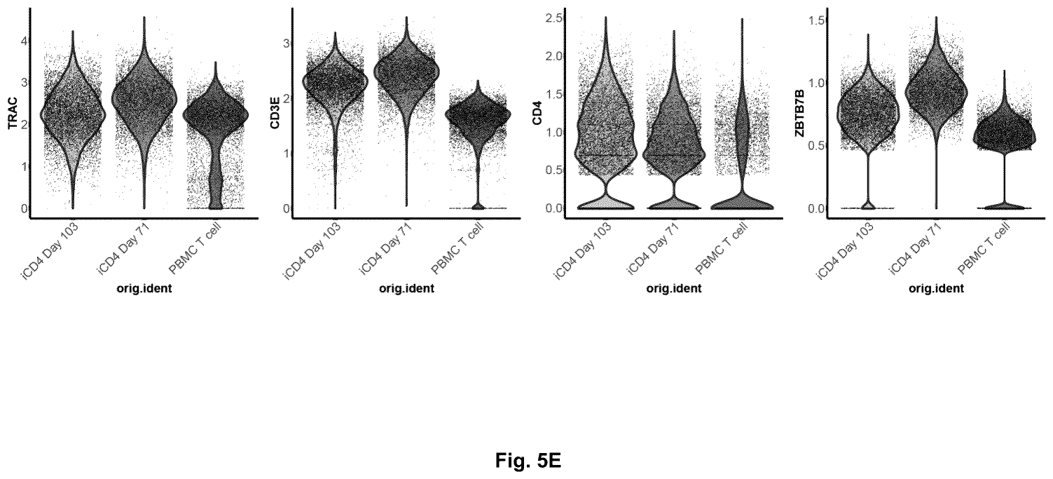

To further demonstrate that the iCD4+ T cells show similarity to conventional TCRab T cells, scRNAseq was performed. iT cells at day 71 and day 103 of culture were sequenced, alongside CD3+ cells isolated from PBMC. Primary T cells were rested overnight in conditions identical to the iT cells and then prepared for sequencing (FIG. 5A). After sequencing, shared nearest neighbor clustering was performed at 0.15 resolution (FIG. 5B).

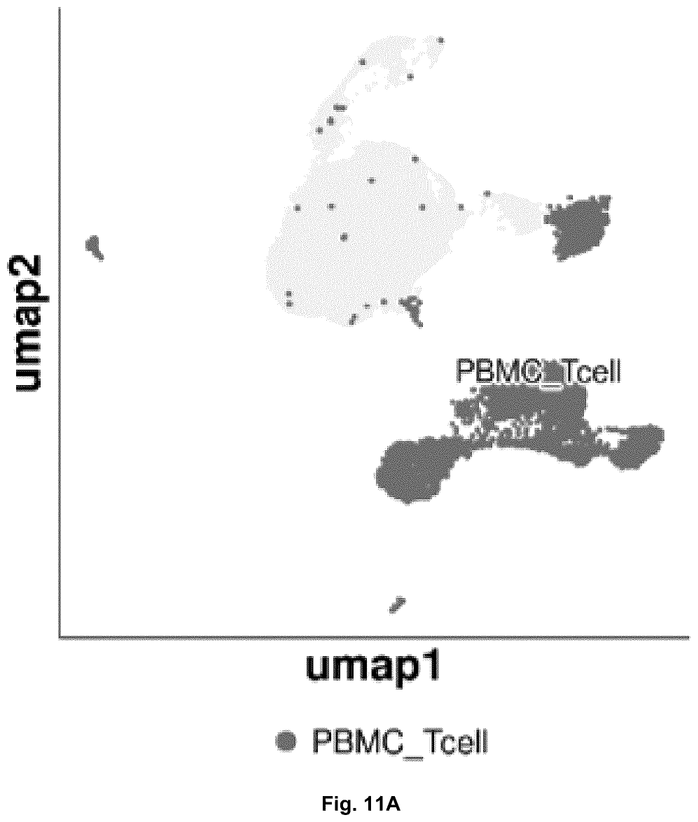

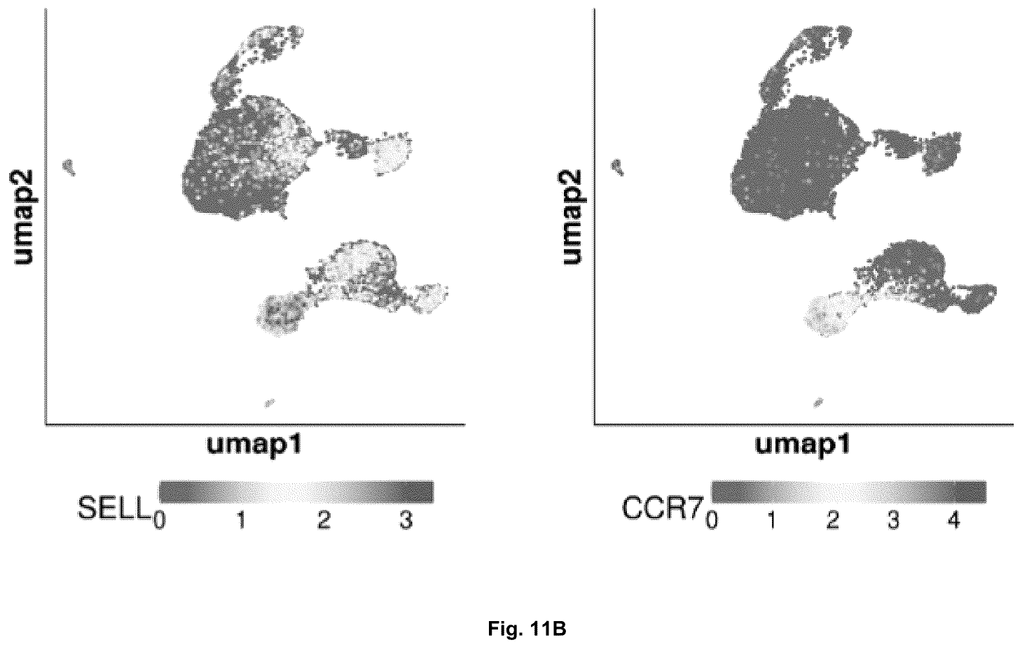

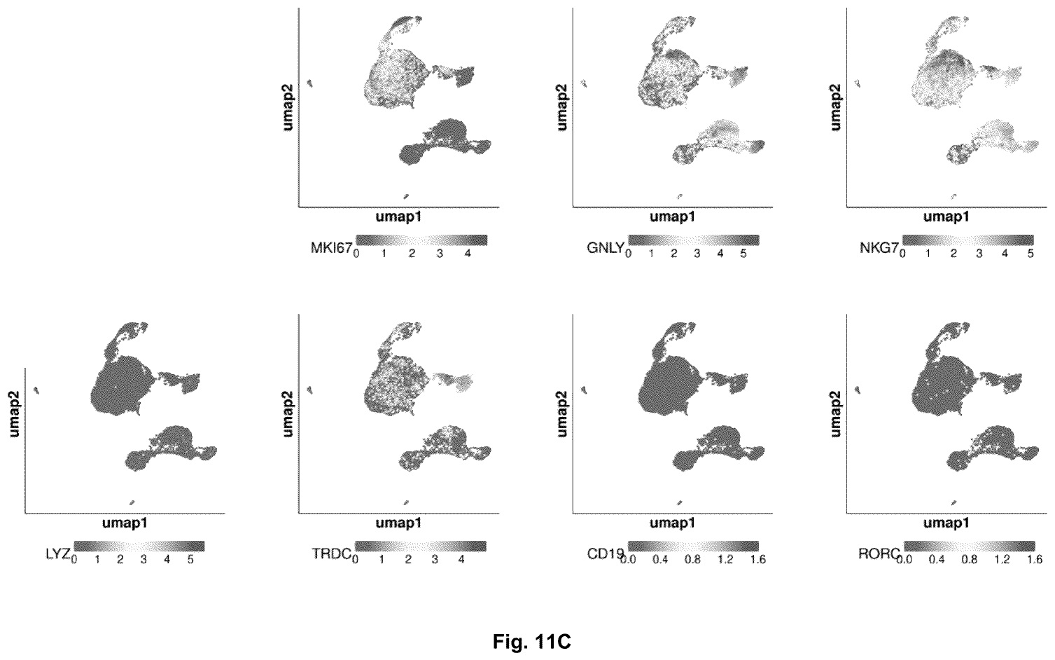

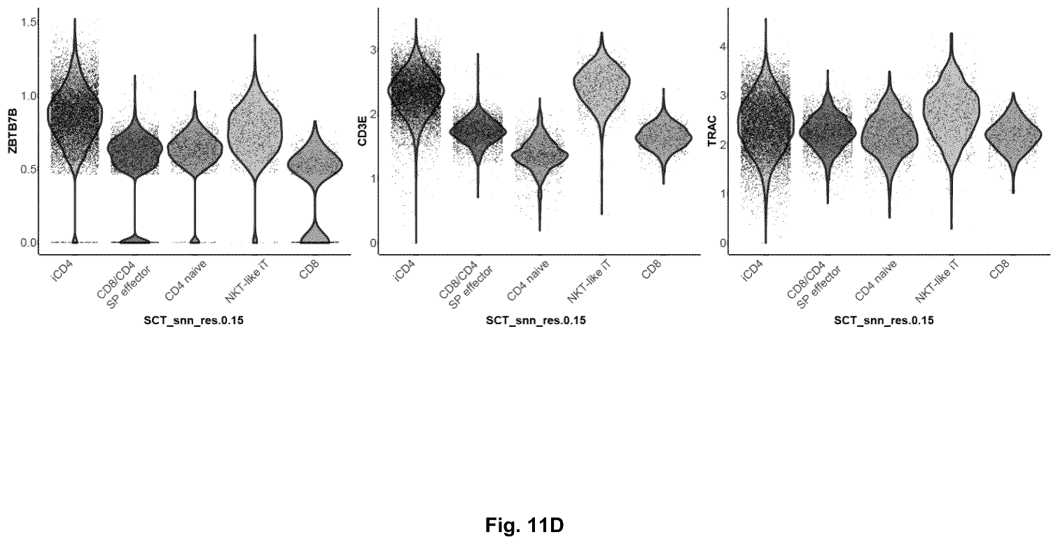

UMAP projection followed by Louvain clustering revealed that despite the iCD4+ T cells being cultured for an additional month with an additional round of stimulation, there was almost complete overlap between day 71 and 103 cells. The PBMC and iT cell samples clustered separately (FIGS. 5B and 11A). Analysis of the differentially expressed genes between the PBMC and iT samples showed that many key drivers behind the differences corresponded to activation states, including CD69 expression and a loss of CD62L which mirrored the flow cytometry results for those markers at day 68 (FIGS. 5C and 11B). There was also significant upregulation of CD40L, classically considered to be a marker of CD4+ T cell activation and differentiation (FIG. 5C).

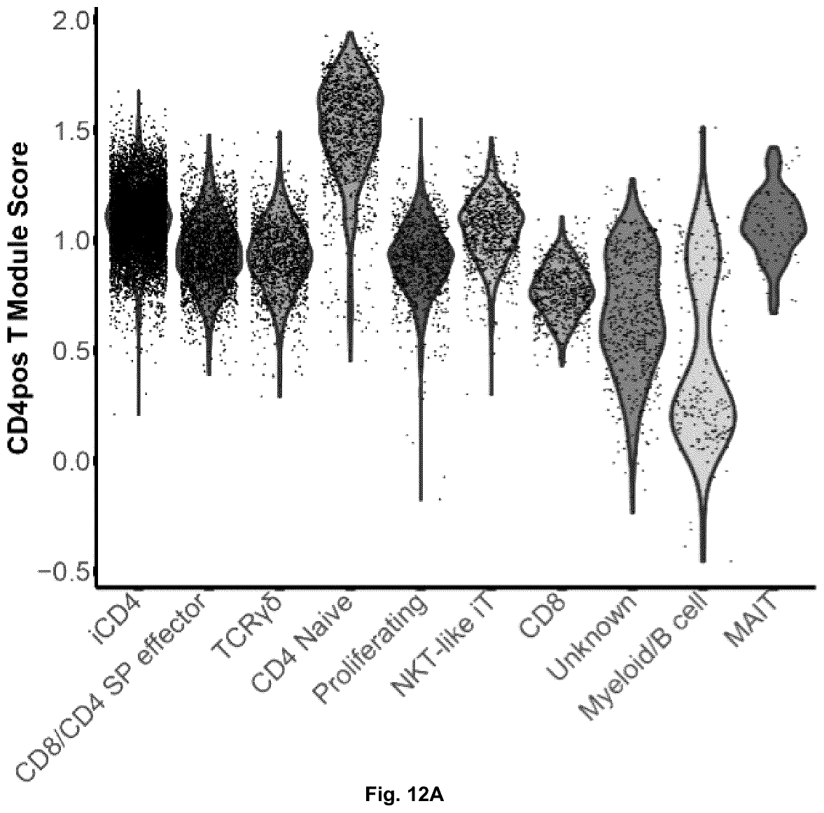

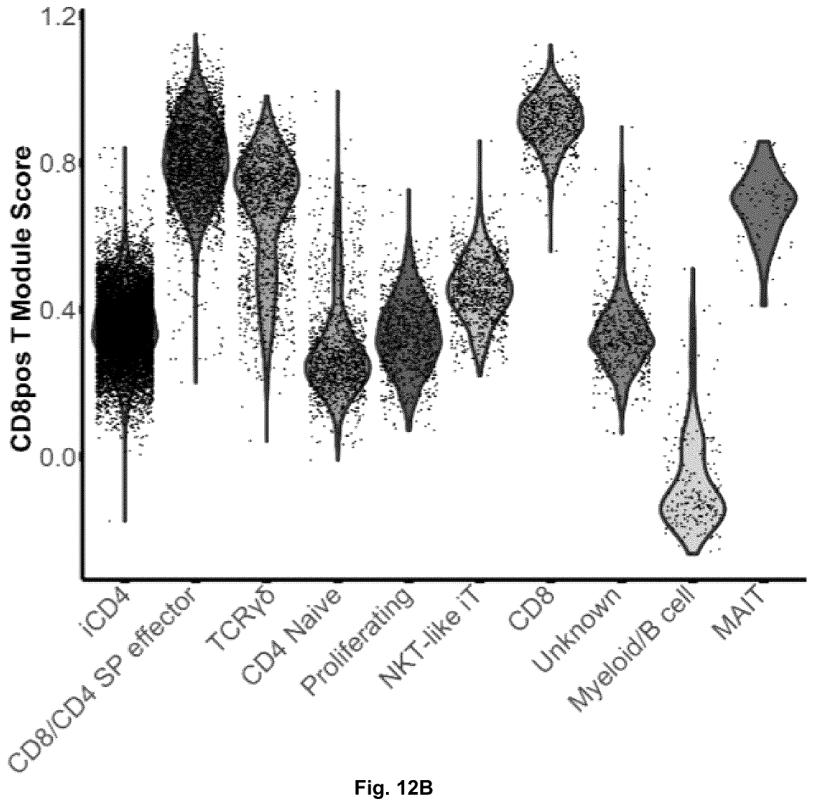

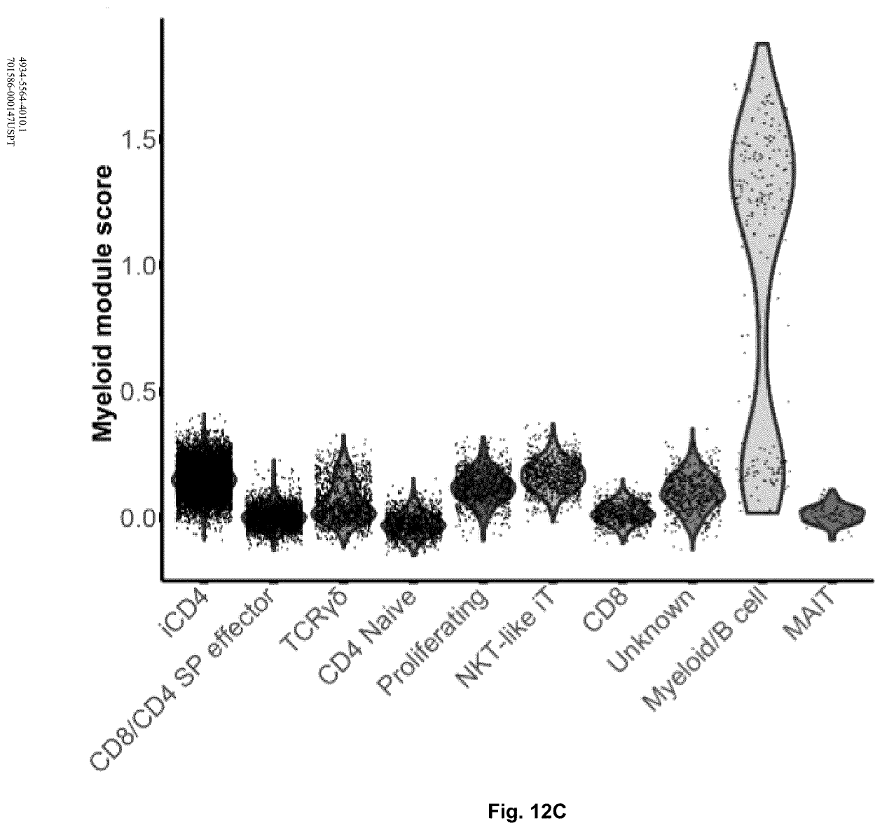

To further classify the identity of the cells, DEG lists generated from a large-scale sequencing study of human PBMCs were used (Terekhova et al., Immunity 57, 188-192, 2024). Displaying the top 25 genes from the CD4+ and CD8+ T cell subsets revealed greater similarity to conventional CD4 single positive TCRab as compared to conventional CD8 single positive cells (FIG. 5D, FIG. 12). This is also reflected by clusters three and six which correspond to CD4 and CD8 single positive T cells, respectively. Using more specific gene lists and expression of key transcription factors, a CD4+ naïve population was identified in the PBMC sample as well as contaminating B cells, macrophages, MAIT cells, CD8 cells and a mixed CD4/CD8 SP cluster with characteristics of effector cells (FIG. 11C) (Garner et al., 2018; Malarkannan 2020; Terekhova et al., 2024). One mixed cluster of iT and PBMC cells likely corresponded to TCRgd cells as they showed expression of the TCRδ constant component (FIG. 11C).

Finally, iCD4+ T cells showed strong expression of ZBTB7B (ThPOK), CD3, TRAC, and CD4 with minimal CD8A and CD8B in the iT samples (FIG. 5E). Expression of TNF and IL4 was also detected across many of the iT cells, further demonstrating the functional potential of the cells even without PMA/ionomycin stimulation conditions.

### DISCUSSION

The robust generation of functional CD4 single positive T cells from human iPSC in a feeder-free manner is accomplished via the removal of Notch signal during TCR mediated DP to SP transition. The resulting iCD4+ T cells proliferate and produce cytokines in response to stimulation. The combination of strength and duration of T cell receptor signaling influences T cell lineage divergence. The downstream effects of Notch ligand withdrawal are investigated herein, including regarding iPSC derived T cells grown in the presence of high concentrations of Notch ligands The absence of Notch ligands allowed for access to the CD4 lineage, which indicates mechanisms blocking access to the CD4 lineages in iPSC T cells. Indeed, this also provides an explanation why until now it has not been possible to access the CD4 lineage from iPSCs, as most previous protocols that relied on T cell maturation conditions containing Notch signaling failed to produce CD4 lineage cells. Strong Notch signaling has been shown to modulate the strength of signal through the T cell receptor in this system, and mechanistic studies can characterize these effects.

CD62L/CCR7 double positivity in a fraction of iCD4+ T cells at day 54 of culture demonstrates the presence of naive T cells present in theiCD4+ T cell cultures at this timepoint. After emigrating from the thymus, T cells continue to develop, spending up to 3 weeks maturing further from a recent thymic emigrant to a naïve state. After stimulation through the TCR, naive CD4+ T cells differentiate into memory cells with distinct functional lineages. Studies can delineate when within the iPSC to iCD4+ T cell platform naive or naive-like cells can be observed. The use of culture conditions containing alternate cytokines, such as IL-7, IL-15 or both in post-specification culture while removing IL-2, provides conditions for the maintenance or expansion of naive cells present in the cultures. The scRNAseq indicates that after long-term culture with expansion periods, iCD4+ T cells acquire effector phenotypes, as reflected by upregulation of CD69, CD40 ligand and downregulation of CCR7. Treatment of naive cells with various cytokines permits polarization of iCD4+ T cells into specific helper subsets, serving as an in vitro screening platform to reveal the molecular pathways responsible for their specification. T regulatory cells can be induced from naive T cells through treatment with TGF-β, IL-2, retinoic acid and rapamycin during CD3/CD28 and can act in a highly anti-inflammatory manner.

The described platform focuses on CD4+ T cell generation in a feeder-free context, addressing a critical issue of important practical implications, which is its capability for scalability and control of the final cell product. The protocol produces cells with clear inflammatory phenotypes including interferon γ production, which can be used in immunotherapy applications.

In summary, a scalable and efficient method is provided for the generation of iPSC-derived CD4+ T cells with functional and expression profiles comparable to those of primary CD4+ T cells. The resulting iCD4+ T cells permit the advancement of iPSC-derived T cell therapies to treat a variety of diseases, particularly those fueled by chronic inflammation.

### Materials and Methods

Described herein is the generation and characterization of functional CD4 lineage T cells from human iPSCs. Using a combination of flow cytometry, mass cytometry, multiplex cytokine detection by LEGENDPLEX and single cell RNA sequencing, the differentiation and identity of iPSC derived CD4+ T cells was investigated in vivo. The differentiation was replicated in 4 iPSC lines representing 3 donors, including both male and female cell lines. Key observations were interrogated using assays at both the protein and RNA level. Experimental parameters and statistical tests are provided in figure legends and relevant sections of Materials and Methods below.

### Tissue Culture Conditions and Cell Lines

Human iPSC lines were maintained on 6-well tissue culture treated plates coated with hESC-qualified Matrigel™ in 2 mL of mTeSR+ media with added Primocin™. Media was changed daily, excepting weekends, where cells were fed twice the standard amount of mTeSR™. Cells were passaged weekly using ReLeSR™ according to manufacturer protocols. Cells were grown at 37° C., 5% CO₂ in standard incubators. iPSC lines used in this study were BU1c2 (XY, EF1a-hSTEMCCA4 loxp lentiviral infection, Cre-excised), BU1c2 TetOn:NICD1, BU2-15-Cr10 TetOn:NICD1 (XY, EF1a-hSTEMCCA4 loxp lentiviral infection, Cre-excised), and BU7 (XX, Sendai virus reprogrammed). Generation of TetOn:NICD1 lines outlined in prior work. Human PBMC cells were isolated by density gradient centrifugation with FICOLL and maintained in X-Vivo® 15 with 50 IU IL-2 added in T75 or larger flasks.

Differentiation from iPSC to Double Positive T Cells

iPSC cells are differentiated to hematopoietic stem and progenitor cells as previously described over a 12-day period (Heinze et al., 2022). CD34+ progenitors are isolated from all floating cells by MACS separation using MILTENYI CD34+ selection kit and LS columns (MILTENYI). CD34+ progenitors are seeded at 5*10⁴ onto TC untreated plates coated with Stemspan™ Lymphoid Differentiation Coating Material as per manufacturer instructions (STEMCELL TECHNOLOGIES). The progenitors are fed with Stemspan™ SFEM II media (STEMCELL TECHNOLOGIES) supplemented with Lymphoid Progenitor Expansion Supplement (STEMCELL TECHNOLOGIES) and Primocin™ After initial seeding in 1 mL of expansion media cells are cultured for 3 days (Day 15), then fed with an additional 1 mL of expansion media. At Day 19 half of the media is aspirated from each well and replaced with fresh media, then cells are moved to a plate with new coating. Half media changes are performed until day 26, when cells are harvested and re-seeded at 0.5 to 1*10\hat{ }6 cells per mL on new plates coated with Stemspan™ Lymphoid Differentiation Coating Material in Stemspan™ SFEM II media supplemented with Lymphoid Progenitor Maturation Supplement (STEMCELL TECHNOLOGIES) and Primocin™. After 3 days maturation media was added as with expansion culture. Half media changes are then performed with maturation media every 3 or 4 days until day 40, when double positive cells were harvested.

Differentiation from DP T Cells to CD4 SP T Cells and SP T Cell Culture Conditions

DP cells were plated on 12 well untreated plates coated with 10 μg of RetroNectin® (TAKARA BIO) at variable density from 1*10\hat{ }5 to 1*10\hat{ }6 cells per mL in Stemspan™ SFEM II media supplemented with Lymphoid Progenitor Maturation Supplement (STEMCELL TECHNOLOGIES) and Primocin™. At the time of plating media is supplemented with an additional 10 ng/mL of IL7 and 5 μg/mL of purified anti-CD3 antibody clone OKT3. The media is then changed as outlined previously with half media changes of Stemspan™ SFEM II media supplemented with Lymphoid Progenitor Maturation Supplement (STEMCELL TECHNOLOGIES), 10 ng/mL of IL7 and Primocin™. At day 54 (14 days after initiation of DP to SP transition) SP T cells can be harvested.

SP iCD4 T cells are moved to a freshly coated RetroNectin® plate, in Stemspan™ SFEM II media supplemented with Lymphoid Progenitor Maturation Supplement (STEMCELL TECHNOLOGIES), 200 U IL-2 and Primocin™. Expansion is initiated with the addition of 25 μg/mL of anti CD3/CD28 Immunocult™ human T cell activator (STEMCELL TECHNOLOGIES). Through the expansion process cells are fed with half media changes as previously described.

At day 68 iT cells begin to be adapted to X-Vivo® 15 media (LONZA) supplemented with 200 U IL-2 by performing half media changes with X-Vivo® 15 media. After this time media is changed every 3 or 4 days by performing a half media change with X-Vivo® 15 media supplemented with 200 U IL-2.

### Conventional Flow Cytometry

Single cell suspensions were harvested from cultures and resuspended in PBS. Where a live/dead dye was used the cells are incubated with fixable viability stain 780 (BD BIOSCIENCES) at 1:1000 for 30 minutes at room temperature in the dark. Cells were then washed with FACS buffer (2% fetal bovine serum in PBS) and a master mix consisting of antibodies detailed in Table 1. Samples were incubated for 30 minutes at room temperature in the dark. All antibodies were used at 1:100 concentration. Stained samples were read on a STRATEDIGM instrument equipped with 405, 488, 552 and 640 nm lasers. Analysis performed in FlowJo™ software.

### Spectral Flow Cytometry

Samples were prepared and stained as outlined in flow cytometry. Master mix contained 1× human Fc block (BD BIOSCIENCES) and 1× BRILLIANT Stain Buffer (BD BIOSCIENCES) following manufacturer instructions. All antibodies were titrated and used at optimal concentration to minimize background staining while retaining signal. Samples were read on a 6 laser CYTEK AURORA instrument and unmixed on the instrument. Analysis performed in FlowJo™ software.

### CyTOF

iPSC derived CD4+ cells were harvested on day 68 and a healthy cryopreserved human PBMC control was thawed. Cells were resuspended in cRPMI at a concentration of 1×10⁶ cells/mL and plated into a 24 W Cell-Repellent Surface Plate at 1 mL per well. Cells were rested for 2 hours at 37° C. Following resting, cells were stimulated with phorbol 12-myristate 13-acetate (PMA)+ionomycin (BIOLEGEND Cell Activation Cocktail, 1:1000 dilution) or left untreated and incubated at 37° C. After 1 hour, Brefeldin A and monensin (BIOLEGEND, 1:1000 dilution) were added to each well and samples were incubated at 37° C. for an additional 18 hours. Following stimulation, cells were harvested and pooled by sample, counted, and washed with PBS. Up to 3×10⁶ cells per sample were washed with Cell Staining Buffer (STANDARD BIOTOOLS) and incubated with FC-receptor blocking solution (BIOLEGEND). Cells were then incubated with the surface antibody cocktail in the presence of Cell-ID 103Rh Intercalator (STANDARD BIOTOOLS) for viability staining. After washing with CSB, cells were fixed with MAXPAR FIX-I Buffer (STANDARD BIOTOOLS), permeabilized with MAXPAR Perm-S Buffer (STANDARD BIOTOOLS) and blocked with sodium heparin blocking solution (SIGMA) in MAXPAR Perm-S Buffer. Cells were then incubated with the intracellular antibody cocktail, washed with CSB and fixed with 1.6% formaldehyde solution in PBS. Cells were resuspended in MAXPAR Fix and Perm buffer supplemented with Cell-ID Intercalator Ir (STANDARD BIOTOOLS) and stored at 2-8° C. overnight prior to acquisition. CyTOF samples were acquired on a CyTOF XT system and samples were normalized with EQ™ SIX ELEMENT Calibration Beads (STANDARD BIOTOOLS) using CyTOF software. Data analysis was conducted using OMIQ (DOTMATICS). Briefly, live singlet cell data from a control PBMC and iCD4 samples were asinh transformed and clustered with PHENOGRAPH algorithm and projected into opt-SNE space (perplexity=30, theta=0.5, opt-SNE endpoint=5000; PCA pre-initialization embedding). Clusters were color-coded and overlaid on the opt-SNE projection graphs. Each marker's mean signal intensities within clustered datasets were organized into hierarchically clustered heatmaps.

### TCR Sequencing

TCR sequencing was accomplished using bulk-immunoprofiling (AZENTA LIFE SCIENCES). This bulk-RNA sequencing platform has amplification of the VDJ regions of the beta TCR chains. Snap-frozen cell pellet samples were submitted from day 68 iPSC-derived T cells. RNA extraction, DNase treatment, cDNA reactions, amplification of the TCR chains, and next generation sequencing were preformed. TCR diversity and V-J usage of the submitted sample was determined.

### Cytokine Quantification

T cell samples were seeded at 1 million cells per mL in X-Vivo® 15 media (LONZA) which had not been supplemented with any cytokines or serum. Phorbol 12-myristate 13-acetate and ionomycin were added at 25 ng/mL and 1 μg/mL concentrations respectively. After a 6 hour incubation each well was centrifuged, and supernatants were harvested and stored at −80 degrees Celsius.

In one instance the cells were also treated with protein transport inhibitor Brefeldin A, and then stained for flow cytometry using EBIOSCIENCES FoxP3/transcription factor staining kit according to manufacturer instructions.

Multiplexed analysis of cytokines was performed using a LEGENDplex™ HU Th Cytokine Panel (12-plex) w/ FP V02 kit from BIOLEGEND. Supernatants were thawed on ice, then diluted in 2 parts assay buffer for each part supernatant. Manufacturer instructions were followed to prepare samples, which were then read on a 6-laser CYTEK AURORA. Data was then analyzed in LEGENDPLEX software (BIOLEGEND).

### Single Cell RNA Sequencing

T cell samples were prepared at day 71 and 103 of culture by using MILTENYI BIOTEC's dead cell removal kit to increase viability. T cells were isolated from peripheral blood mononuclear cells using a CD3 magnetic separation kit (MILTENYI). After magnetic separation all samples had a viability above 90%.

Samples were processed for sequencing by a Single Cell Sequencing core, using a 10× GENOMICS 3′v4 kit. Prepared libraries were sequenced using ILLUMINA NEXTSEQ 2000 Next Generation Sequencing, with a P3 flow cell for 100 cycles.

Sequencing files were mapped to the human genome reference (GRCh37) using CELLRANGER v3.0.2. SEURAT v3.2.3 was used for downstream analysis and quality control. After inspection of the quality control metrics, cells with 15% to 35% of mitochondrial content and <800 detected genes were excluded for downstream analyses. In addition, doublets were also excluded for downstream analysis. Normalization and scaling of the unique molecular identifier (UMI) counts were performed using the regularized negative binomial regression (SCTransform). Following the standard procedure in Seurat's pipeline linear dimensionality reduction (principal component analysis) was conducted, and the top 20 principal components were used to compute the unsupervised Uniform Manifold Approximation and Projection (UMAP). For clustering of the cells, the Louvain algorithm was applied at a range of resolutions from 1.5 to 0.05 (more to fewer clusters). Populations were annotated using Louvain Clustering at a resolution of 0.05. Cell cycle scores and classifications were done using the Seurat's cell-cycle scoring and regression method. Cluster specific genes were calculated using MAST framework in SEURAT wrapper. ALRA's algorithm was used to impute the data, to correct for false zeros counts. An online Shiny app has been established to allow interactive, user-friendly visualizations of gene expression in each population, crem-bu.shinyapps.io/24_09_03_Julian/ along with crem-bu.shinyapps.io/24_09_03_Julian_imputed/ for the imputed data.

Single cell RNA sequencing data is available on the GEO repository under accession number GSE279734. All other raw data and code can be provided upon request.

TABLE 1
Materials

| REAGENT or RESOURCE | SOURCE | IDENTIFIER |
|---|---|---|
| Antibodies | | |
| BV421 Mouse Anti-Human CD34 | BD BIOSCIENCE | Cat#562577 |
| PE Mouse Anti-Human CD56 | BIOLEGEND | Cat#304605 |
| BV605 Mouse Anti-Human CD5 | BD BIOSCIENCE | Cat#563945 |
| AF647 Mouse Anti-Human HLA-DR, DP, DQ | BD BIOSCIENCE | Cat#563591 |
| BV421 Mouse Anti-Human CD1a | BD BIOSCIENCE | Cat#563939 |
| BB515 Mouse Anti-Human CD4 | BD BIOSCIENCE | Cat#564500 |
| APC Mouse Anti-Human CD7 | BD BIOSCIENCE | Cat#561604 |
| FITC Mouse Anti-Human CD16 | BD BIOSCIENCE | Cat#561308 |
| APC Mouse Anti-Human CD69 | BD BIOSCIENCE | Cat#560967 |
| BV421 Mouse Anti-Human CD8 | BD BIOSCIENCE | Cat#562428 |
| BUV395 Mouse Anti-Human CD8b | BD BIOSCIENCES | Cat# 742396 |
| LD Blue Viability | THERMO FISHER | Cat# L23105 |
| BUV496 Mouse Anti-Human CD16 | BD BIOSCIENCES | Cat# 612945 |
| BUV737 Mouse Anti-Human CD56 | BD BIOSCIENCES | Cat# 612767 |
| BUV805 Mouse Anti-Human CD8 | BD BIOSCIENCES | Cat# 612890 |
| BV421 Mouse Anti-Human CD69 | BIOLEGEND | Cat# 310929 |
| FITC Mouse Anti-Human TCR γδ | BD BIOSCIENCES | Cat# 559878 |
| BV480 Mouse Anti-Human CD7 | BD BIOSCIENCES | Cat# 566161 |
| BV510 Mouse Anti-Human CD33 | BIOLEGEND | Cat# 366609 |
| BV570 Mouse Anti-Human CD14 | BIOLEGEND | Cat# 301831 |
| BV605 Mouse Anti-Human TCR a/b | BIOLEGEND | Cat# 306731 |
| SuperBright ™ 645 Rat Anti-Mouse NKp46 | THERMO FISHER | Cat# 64-3351-80 |
| (CD335) | | |
| BV711 Mouse Anti-Human CD5 | BD BIOSCIENCES | Cat# 563170 |
| BV785 Mouse Anti-Human CD337 (NKp30) | BIOLEGEND | Cat# 325229 |
| Spark Blue ™ 550 Mouse Anti-Human CD3 | BIOLEGEND | Cat# 344851 |
| PE Mouse Anti-Human CD25 | THERMO FISHER | Cat# 12-0257-41 |
| PE-Cy7 Mouse Anti-Human CD336 (NKp44) | BIOLEGEND | Cat# 325115 |
| AF647 Mouse Anti-Human CD1a | BIOLEGEND | Cat# 300116 |
| APC-H7 Mouse anti-Human CD45 | BD BIOSCIENCES | Cat# 560274 |
| APC-Fire810 Mouse anti-Human CD4 | BIOLEGEND | Cat# 344661 |
| Human BD Fc Block | BD BIOSCIENCES | Cat# 564220 |
| PerCP/Cyanine5.5 Mouse Anti-Human CD24 | BIOLEGEND | Cat# 311115 |
| BUV615 Mouse Anti-Human CD62L | BD BIOSCIENCES | Cat# 751364 |
| PE/Dazzle ™ 594 Mouse Anti-Human HLA- | BIOLEGEND | Cat# 311439 |
| A, B, C | | |
| PE-Cy7 Mouse Anti-Human CCR7 (CD197) | BD BIOSCIENCES | Cat# 567314 |
| Biological Samples | | |
| Peripheral Blood Mononuclear cells | | |
| Chemicals, Peptides, and Recombinant Proteins | | |
| Recombinant Human BMP-4 Protein | R&D SYSTEMS | Cat# 314-BP |
| Recombinant Human VEGF 165 Protein | R&D SYSTEMS | Cat#293-VE |
| Recombinant Human Wnt-3a Protein | R&D SYSTEMS | Cat#5036-WN |
| Recombinant Human FGF basic/FGF2 (146 aa) | R&D SYSTEMS | Cat#233-FB |
| Protein | | |
| CHIR99021 | REPROCELL | Cat#04-0004 |
| Recombinant Human SCF Protein | R&D SYSTEMS | Cat#255-SC |
| Recombinant Human Sonic Hedgehog (Shh) | PEPROTECH | Cat#100-45 |
| Recombinant Human FLT3L | BIO X CELL | No longer made |
| Recombinant Human TPO | R&D SYSTEMS | Cat#288-TP |
| Recombinant Human IL-6 Protein, CF | R&D SYSTEMS | Cat#206-IL CF |
| Recombinant Human IL-3 Protein | R&D SYSTEMS | Cat#203-IL |
| Recombinant Human IL-11 Protein | THERMO FISHER | Cat#200-11 |
| Recombinant Human IGF-I/IGF-1 Protein, CF | R&D SYSTEMS | Cat#291-G1 |
| Human Transferrin | SIGMA-ALDRICH | Cat#10652202001 |
| Recombinant Human IL-7 Protein | R&D SYSTEMS | Cat#207-IL |
| hESC Matrigel ™ Matrix | CORNING | Cat#354277 |
| Matrigel ™ Matrix | CORNING | Cat#354234 |
| Stemolecule ™ Y27632 | REPROCELL | Cat#04-0012-02 |
| Ascorbic Acid | SIGMA | Cat#A4544 |
| mTeSR ™1 Media | STEMCELL | Cat#85850 |
| | TECHNOLOGIES | |
| mTeSR ™ Plus Media | STEMCELL | Cat#100-0276 |
| | TECHNOLOGIES | |
| StemSpan ™ Lymphoid Differentiation Coating | STEMCELL | Cat# 09925 |
| Material (100X) | TECHNOLOGIES | |
| StemSpan ™ SFEM II | STEMCELL | Cat# 09655 |
| | TECHNOLOGIES | |
| StemSpan ™ Lymphoid Progenitor Expansion | STEMCELL | Cat# 09915 |
| Supplement (10X) | TECHNOLOGIES | |
| Stem Span ™ T Cell Progenitor Maturation | STEMCELL | Cat# 09930 |
| Supplement | TECHNOLOGIES | |
| X-VIVO ® 15 Serum-free Hematopoietic Cell | LONZA | Cat# 02-053Q |
| Medium | | |
| StemPro ®-34 SFM | GIBCO | Cat#10640-019 |
| GlutaMax ™ (100x) | GIBCO | Cat#35050-061 |
| 1-thioglycerol | SIGMA | Cat#M6145 |
| ReLeSR ™ | STEMCELL | Cat#05873 |
| | TECHNOLOGIES | |
| Primocin ™ | INVIVOGEN | Ant-pm-2 |
| Doxycycline | SIGMA | Cat# D3072 |
| FVS780 | BD BIOSCIENCES | Cat# 565388 |

TABLE 2
Panel and titers for spectral flow cytometry.

| Number | Fluorophore | Antibody | Titer | Catalog number |
|---|---|---|---|---|
| 1 | BUV395 | CD8b | 1:50 | 742396 |
| 2 | BUV496 | CD16 | 1:50 | 612945 |
| 3 | BUV615 | CD62L | 1:400 | 751364 |
| 4 | BUV737 | CD56 | 1:100 | 612767 |
| 5 | BUV805 | CD8a | 1:200 | 612890 |
| 6 | BV421 | CD69 | 1:100 | 310929 |
| 7 | BV480 | CD7 | 1:50 | 566161 |
| 8 | BV510 | CD33 | 1:100 | 366609 |
| 9 | BV570 | CD14 | 1:50 | 301831 |
| 10 | BV605 | TCRab | 1:50 | 306731 |
| 11 | BV711 | CD5 | 1:200 | 563170 |
| 12 | BV785 | NKp30 | 1:50 | 325229 |
| 13 | SPARK BLUE | CD3 | 1:100 | 344851 |
| | 550 | | | |
| 14 | SUPERBRIGHT | NKp46 CD335 | 1:50 | 64-3351-80 |
| | 645 | | | |
| 15 | FITC | TCRgd | 1:50 | 130-114-029 |
| 16 | PE | CD25 | 1:50 | 12-0257-41 |
| 17 | PE-Dazzle594 | MHC-I | 1:800 | 311439 |
| 18 | PE-Cy7 | CCR7 | 1:100 | 567314 |
| 19 | AF647 | CD1a | 1:100 | 300116 |
| 20 | APCH7 | CD45 | 1:800 | 560274 |
| 21 | APC-Fire810 | CD4 | 1:400 | 344661 |
| 22 | PerCP-Cy5.5 | CD24 | 1:25 | 311115 |
| 23 | LD Blue Viability | Viability | 1:1000 | L23105 |

TABLE 3
Panel used for CyTOF study.

| Number | Metal | Marker | Clone |
|---|---|---|---|
| 1 | 149 Sm | CD56 | NCAM16.2 |
| 2 | 143 Nd | CD45RA | H100 |
| 3 | 153 Eu | CD25 | BC96 |
| 4 | 155 Gd | CD27 | L128 |
| 5 | 160 Gd | CD28 | CD28.2 |
| 6 | 163 Dy | CD45RO | UCHL1 |
| 7 | 168 Er | CD127 | A019D5 |
| 8 | 164 Dy | TGFb | S20006A |
| 9 | 156 Gd | PD-1 | EH12.2H7 |
| 10 | 166 Er | TIGIT | MBSA43 |
| 11 | | 103Rh Viability | |
| 12 | 110 Cd | IL-3 | BVD3-1F9 |
| 13 | 113 Cd | MIP1a | W16009B |
| 14 | 139 La | IL-8 | G265-8 |
| 15 | 152 Sm | MIP1b | D21-1351 |
| 16 | 162 Dy | Amphiregulin | AREG559 |
| 17 | 173 Yb | IL-1b | CRM56 |
| 18 | 144 Nd | CD4 | SK3 |
| 19 | 112 Cd | IL-2 | MQ1-17H12 |
| 20 | 114 Cd | TNFa | MAb11 |
| 21 | 116 Cd | IFN-y | B27 |
| 22 | 147 Sm | IL-5 | TRFK5 |
| 23 | 150 Nd | IL-22 | HI100 |
| 24 | 165 Ho | IL-10 | JES3-9D7 |
| 25 | 169 Tm | IL-13 | JES10 |
| 26 | 171 Yb | IL-4 | MP4-25D2 |
| 27 | 172 Yb | IL-21 | 3A3-N2 |
| 28 | 195 Pt | IL-17A | BL168 |
| 29 | 198 Pt | Granzyme B | GB11 |
| 30 | 142 Nd | CD8 | SK1 |
| 31 | 170 Er | CD3 | UCHT1 |

What is claimed herein is:

1. A method of producing a population of CD4+CD8− single-positive (SP) T cells, the method comprising:
culturing a population of CD4+CD8+ double positive (DP) T progenitor cells in the absence of Notch ligand and an anti-CD28 agent, and in a medium comprising interleukin-7 (IL-7) and an anti-CD3 agent until the population of DP T progenitor cells differentiates into the population of CD4+CD8− SP T cells.

2. The method of claim 1, wherein the anti-CD3 agent specifically binds and activates CD3 intracellular signalling.

3. The method of claim 1, wherein the anti-CD3 agent is not bound to a substrate.

4. The method of claim 1, wherein the anti-CD3 agent comprises an anti-CD3 antibody or an antigen binding domain thereof, or an anti-CD3 aptamer.

5. The method of claim 4, wherein the anti-CD3 antibody is selected from OKT3, Otelixizumab, Teplizumab, HIT3a, UCHT1, 17A2, 145-2C11, or Foralumab.

6. The method of claim 4, wherein the anti-CD3 antibody is OKT3.

7. The method of claim 1, wherein the anti-CD3 agent is present at a concentration of about 5 μg/mL.

8. The method of claim 1, wherein the IL-7 is present at a concentration of about 10 ng/ml.

9. The method of claim 1, wherein the population of DP T progenitor cells are cultured for at least 14 days.

10. The method of claim 1, wherein the population of DP T progenitor cells are cultured with a substrate coated with fibronectin or a recombinant variant thereof.

11. The method of claim 10, wherein the fibronectin or recombinant variant thereof is coated on the substrate at a concentration of at least 10 μg/mL.

12. The method of claim 1, further comprising culturing the population of CD4+CD8− SP T cells in a medium comprising: the anti-CD3 agent, the anti-CD28 agent, and interleukin-2 (IL-2).

13. The method of claim 12, wherein the anti-CD28 agent specifically binds and activates CD28 intracellular signalling.

14. The method of claim 12, wherein the anti-CD28 agent comprises an anti-CD28 antibody or an antigen binding domain thereof, or an anti-CD28 aptamer.

15. The method of claim 12, wherein the anti-CD3 agent and the anti-CD28 agent are comprised by a single bispecific agent, are linked to a substrate, and/or are in a tetrameric complex.

16. The method of claim 12, wherein the anti-CD3 agent and the anti-CD28 agent comprise anti-CD3 antibodies and anti-CD28 antibodies in tetrameric complexes.

17. The method of claim 12, wherein the anti-CD3 agent and the anti-CD28 agent are present at a concentration of about 25 μg/mL.

18. The method of claim 12, wherein the IL-2 is present at a concentration of about 200 U/mL.

19. The method of claim 12, wherein the culturing increases proliferation of the population of CD4+CD8− SP T cells.

20. The method of claim 1, wherein the population of CD4+CD8− SP T cells is cultured for at least 14 days to at most 140 days.

21. The method of claim 1, wherein the population of CD4+CD8− SP T cells comprises at least 40%, at least 50%, at least 60%, or more CD4+CD8− SP T cells; and/or wherein the population of CD4+CD8− SP T cells comprises at least 1×10⁷, at least 1×10⁸, at least 1×10⁹ or more total CD4+CD8− SP T cells.

22. The method of claim 1, wherein the CD4+CD8− SP T cells are CD3+ and TCRαβ+; wherein the CD4+CD8− SP T cells are CD62L+, CD5+, CCR7+, MHC I+, CD69+, and/or CD25+; wherein the CD4+CD8− SP T cells express T-helper-inducing POZ-Kruppel-like factor (ThPOK) and/or T cell receptor alpha chain constant (TRAC); wherein the CD4+CD8− SP T cells are capable upon stimulation of secreting at least one of the following: interferon-γ (IFNγ), tumor necrosis factor-α (TNFα), interleukin-2 (IL-2), interleukin 4 (IL-4), interleukin 5 (IL-5), interleukin 6 (IL-6), interleukin 8 (IL-8), interleukin 9 (IL-9), interleukin 10 (IL-10), interleukin 13 (IL-13), interleukin 17a (IL-17a), interleukin 17f (IL-17f), interleukin 22 (IL-22), amphiregulin (AREG), granzyme B, macrophage inflammatory protein-1 alpha (MIP1α), and/or macrophage inflammatory protein-1 beta (MIP1β), or any combination thereof; and/or wherein at least 90% of the CD4+CD8− SP T cells in the population are capable upon stimulation of secreting IFNγ and TNFα.

23. The method of claim 1, further comprising, prior to culturing the population of DP T progenitor cells, a step of: culturing a population of CD34+ hematopoietic stem and progenitor cells (HSPCs) on a Notch ligand-coated substrate in a medium comprising interleukin-7 (IL-7), until the population of CD34+ HSPCs differentiates into the population of DP T progenitor cells.

24. The method of claim 23, further comprising, prior to culturing the population of CD34+ HSPCs, a step of: culturing a population of pluripotent stem cells in a medium comprising: bone morphogenetic protein-4 (BMP-4); vascular endothelial growth factor (VEGF); and a glycogen synthase kinase-3β inhibitor, until the population of pluripotent stem cells differentiates into the population of CD34+ HSPCs.

25. The method of claim 24, further comprising, prior to culturing the population of pluripotent stem cells, genetically modifying the population of pluripotent stem cells to express a polypeptide exogenous to the pluripotent stem cell.

26. The method of claim 25, wherein the polypeptide comprises a chimeric antigen receptor (CAR).

27. A population of CD4+CD8− SP T cells produced using the method of claim 25.

28. A composition comprising the population of claim 27.

29. A pharmaceutical composition comprising the population of claim 27 and a pharmaceutically acceptable carrier.

30. A method of treating an autoimmune disease, cancer, or infectious disease, the method comprising administering an effective amount of the population of claim 27 to a subject in need thereof.


SEQUENCE LISTING (10 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 3 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 4 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 6 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 7 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 10 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV